(19) United States
(12) Patent Application Publication
(10) Pub. No.: US 2026/0055114 A1
(43) Pub. Date: Feb. 26, 2026

(54) IMIDAZO[1,2-B]PYRIDAZINE INHIBITORS OF CYCLIN-DEPENDENT KINASES

(71) Applicant: H. LEE MOFFITT CANCER CENTER AND RESEARCH INSTITUTE, INC., Tampa, FL (US)

(72) Inventors: Andrii MONASTYRSKYI, Lutz, FL (US); Derek DUCKETT, Land O Lakes, FL (US); Thiyagumurthy PANDURANGAN, Tampa, FL (US)

(21) Appl. No.: 19/104,584

(22) PCT Filed: Aug. 17, 2023
(86) PCT No.: PCT/US2023/030448

§ 371 Date: Feb. 18, 2025

Related U.S. Application Data
(60) Provisional application No. 63/399,012, filed on Aug. 18, 2022.

(51) Int. Cl. C07D 487/04; A61K 31/5025; A61K 31/5377; C07D 519/00

(52) U.S. Cl. CPC C07D 487/04; A61K 31/5025; A61K 31/5377; C07D 519/00

(57) ABSTRACT

Disclosed are inhibitors for cyclin-dependent kinase 12 or 13 (CDK 12/13) of Formula I comprising imidazo [1.2-b]pyridazine core, and uses thereof. Methods of using the compounds, or pharmaceutical compositions comprising compounds, to treat cancers or inflammatory or myotonic dystrophy type I diseases in mammals, are also disclosed.

### CROSS-REFERENCE TO RELATED APPLICATIONS

This application claims the benefit of priority to U.S. Provisional Patent Application No. 63/399,012 filed Aug. 18, 2022, the disclosure of which is incorporated herein by reference in its entirety.

### TECHNICAL FIELD

This disclosure relates to compounds useful in treating medical disorders, and more particularly to imidazo[1,2-b]pyridazine inhibitors of CDK12 and/or CDK13 useful in treating cell proliferative disorders.

### BACKGROUND

There is a continuing need to identify and develop new chemical entities for treating proliferative diseases and conditions including cancers and myotonic dystrophy. Among the targets for potential anti-proliferative compounds under investigation are the group of enzymes known as protein kinases. Cyclin-dependent kinases (CDKs) are a family of protein serine/threonine kinases that are regulated and activated by post translational modification such as, phosphorylation and require interaction with noncatalytic regulatory subunits known as cyclins for their activities (Genome Biol 2014, 15 (6), 122). In mammalian cells, CDKs control transcription and other important cellular functions, including cell proliferation. So far, more than 20 CDKs and 30 cyclins have been reported. Although individual CDKs have multiple cellular roles, CDKs 1-7 have been best characterized in regulating cell cycle progression whereas, CDKs 7-20 have demonstrated roles in transcription and co-transcriptional processes (Development 2013, 140 (15), 3079-3093: Malumbres M (2014) cyclin dependent kinases. Genome Biol).

Dysregulation of cell cycle control and transcription are hallmarks of cancer. Genetic alterations including amplification, overexpression and mutation of CDKs or their cognate cyclins contribute to unscheduled proliferation as well as genomic and chromosomal instability; and aberrant activity of CDKs is a common feature of most cancer types. Therefore, CDKs constitute biomarkers of proliferation and attractive pharmacological targets for development of anticancer drugs (Cancers 2015, 7 (1), 179-237; Nat Rev Cancer 2009, 9 (3), 153-66). Indeed, three CDK inhibitors targeting the cell cycle regulatory CDKs, CDK4 and CDK6, are approved in hormone receptor-positive metastatic breast cancer, and several inhibitors targeting the transcriptional CDKs, CDK7, CDK8/19 and CDK9, are currently in clinical trials for the treatment of a wide variety of malignancies (Pharmacol Res 2020, 152, 104609; Transcription 2019, 10 (2), 118-136).

Cyclin-dependent kinases 12 and 13 (CDK12/13) belong to the group of transcriptional CDKs and in association with their cognate cyclin, cyclin K, promote transcription elongation through phosphorylation of Ser2 and Ser5 within the YSPTSPS heptad repeats of the carboxy-terminal domain (CTD) of RNA Polymerase II (Pol II) with higher activities when Ser7 is pre-phosphorylated (Cell Div 2012, 7, 12; Nat Commun 2014, 5, 3505). Moreover, unlike other transcriptional CDKs, both CDK12 and CDK13 possess N-terminal arginine-serine-rich domains characteristic of RNA binding and splicing factors and have been shown to directly interact with splicing factors and regulate splicing and 3′-end processing. In fact, loss of CDK12/13, or cyclin K, impedes both global Pol II processivity and pre-mRNA processing, suggesting redundancy of the two kinases as fundamental regulators of global Pol II processivity and transcription elongation (Mol Cell Biol 2015, 35 (6), 928-38; Science Advances 2020, 6 (18), eaaz5041).

Upon loss of CDK12 activity, defects in elongation due to premature cleavage and termination as a result of the activation of intronic polyadenylation (IPA) sites that are enriched in longer genes, including DDR genes were observed (Dubbury S. J et al (2018) Nature). Thus, the resulting DDR defect (or BRCA-like phenotype) in CDK12 mutant tumors might present an opportunity for development of ‘synthetic-lethal’ therapeutic strategies. Indeed, ovarian and prostate cancers with functional mutations in CDK12 elicite a BRCAness phenotype with associated hypersensitivity to poly(adenosine diphosphate-ribose) polymerase (PARP) inhibitors and DNA cross-linking agents (Journal of Biological Chemistry 2014, 289 (13), 9247-9253; Nucleic Acids Research 2015, 43 (5), 2575-2589; Cancer Research 2014, 74 (1), 287-97; Cancer Cell 2018, 33 (2), 202-216 e6).

In addition, tumors driven by the so called “undruggable” oncogenes controlled by super-enhancers such as MYC and EWS/FLI are highly dependent on transcription and DDR gene expression for their rapid replication. Thus, inhibiting the function of CDK12 as both a transcriptional coactivator and a regulator of DNA damage related proteins is synthetically lethal with MYC overexpression (in neuroblastoma) and EWS/FLI expression (in Ewing sarcoma) (Nature 2011, 474 (7353), 609-615; Nat Med 2019, 25 (10), 1526; P Natl Acad Sci USA 2012, 109 (24), 9545-9550). Furthermore, in human epidermal growth factor receptor (HER) 2-positive breast, gastric and papillary thyroid cancer a strong correlation between CDK12 level and high tumor grade exists (BMC Cancer 2010, 10, 539; Theranostics 2020, 10 (14), 6201-6215; Journal of Cancer 2020, 11 (15), 4308-4315). Studies also propose that CDK12-mediated cell cycle vulnerabilities in cancer exist and are amenable for CDK12 targeted therapy (Nat Commun 2018, 9 (1), 1876; EMBO Rep 2019, 20 (9), e47592). The role of CDK13 in transcription is less well understood, and CDK13 mutations have not been reported in cancer; except for its amplification in hepatocellular carcinoma (HCC), where CDK13 copy number was significantly associated with clinical onset of HCC (PLOS One 2012, 7 (8), e43223). Taken together, these pre-clinical studies propose that selective CDK12 and/or CDK13 inhibitors have potential use as single agents or in combination therapy in cancer. In addition, recent studies have expanded the suitability of targeting CDK12 to inflammatory (Science Signaling 2018, 11 (541), eaam8216) and myotonic dystrophy type 1 diseases (Science Translational Medicine 2020,12 (541), eaaz2415).

New and potent compounds are needed that inhibit the activity of CDK12 and/or CDK13, particularly for the prevention or treatment of proliferative diseases and conditions, including cancers and myotonic dystrophy. The compositions and methods disclosed herein address these and other needs.

### SUMMARY

The present disclosure provides substituted imidazo[1,2-b]pyridazine derivatives of Formula (I), and pharmaceutically acceptable salts, solvates, hydrates, polymorphs, co-crystals, tautomers, stereoisomers, isotopically labeled derivatives, prodrugs, and compositions thereof. Also provided are methods involving the inventive compounds or compositions for treating and/or preventing cell proliferative diseases including certain cancers of breast, brain, ovarian, lung, colorectal cancer, leukemias, lymphoma, melanoma, multiple myeloma, Ewing's sarcoma, osteosarcoma and inflammatory and myotonic dystrophy type 1 diseases in a mammal. Treatment of a subject with a proliferative disease using a compound or composition of the invention may inhibit the aberrant activity of kinases, such as a cyclin-dependent kinases (CDK) (e.g., CDK12/13), and therefore, induce potent antiproliferative and apoptotic effects and/or inhibit transcription in the subject.

In one aspect, a compound is provided of Formula I

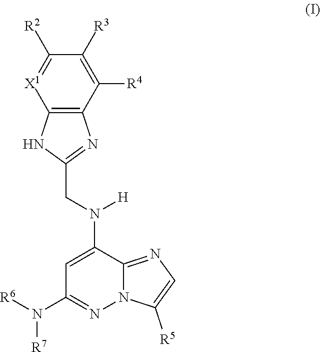

or a pharmaceutically acceptable salt or derivative thereof;
wherein all variables are as further defined herein.

In a second aspect, a pharmaceutical composition is provided comprising a compound described herein, or a pharmaceutically acceptable salt or derivative thereof, and a pharmaceutically acceptable carrier or excipient.

In a further aspect, a method is provided of inhibiting a cyclin-dependent kinase, comprising contacting the cyclin dependent-kinase with an effective amount or concentration of a compound described herein, or a pharmaceutically acceptable salt or derivative thereof, or a pharmaceutical composition described herein.

In another aspect, a method is provided of treating a disorder of uncontrolled cellular proliferation in a mammal comprising administering to the mammal an effective amount of a compound described herein, or a pharmaceutically acceptable salt thereof, or a pharmaceutical composition described herein.

In another aspect, a method is provided for the treatment of a disease selected from inflammatory or myotonic dystrophy type diseases in a mammal comprising administering to the mammal an effective amount of a compound described herein, or a pharmaceutically acceptable salt thereof, or a pharmaceutical composition described herein.

The details of one or more aspects of the disclosure are set forth in the description below. Other features, objects, and advantages of the disclosure will be apparent from the description and the claims.

### DETAILED DESCRIPTION

The following description of the disclosure is provided as an enabling teaching of the disclosure in its best, currently known aspects. Many modifications and other aspects disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual aspects described and illustrated herein has discrete components and features which may be readily separated from or combined with the features of any of the other several aspects without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound or composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a cell proliferative disorder. The effect can be therapeutic in terms of a partial or complete cure of a disease, condition, symptom or adverse effect attributed to the disease, disorder, or condition. The term “treatment” as used herein can include any treatment of a disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

### Chemical Definitions

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context. It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

The terms for functional groups as used herein are not intended to be limited to monovalent radicals and may include polyvalent radical groups as appropriate, such as divalent, trivalent, tetravalent, pentavalent, and hexavalent groups, and the like, based on the position and location of such groups in the compounds described herein as would be readily understood by the skilled person.

“Alkyl” is a straight chain or branched saturated aliphatic hydrocarbon group. In certain aspects, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl) C₀-C₄alkyl, or —C₀-C₄ (C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one aspect, the alkyl group is optionally substituted as described herein.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In one aspect, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one aspect, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one aspect, the alkynyl group is optionally substituted as described herein.

“Alkoxy” is an alkyl group as defined above covalently bound through an oxygen bridge (—O—). Examples of alkoxy include, but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—). In one aspect, the alkoxy group is optionally substituted as described herein.

“Alkanoyl” is an alkyl group as defined above covalently bound through a carbonyl (C═O) bridge. The carbonyl carbon is included in the number of carbons, for example C₂alkanoyl is a CH₃(C═O)— group. In one aspect, the alkanoyl group is optionally substituted as described herein.

“Halo” or “halogen” indicates, independently, any of fluoro, chloro, bromo or iodo.

“Aryl” indicates an aromatic group containing only carbon in the aromatic ring or rings. In one aspect, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B. P, Si and S, to form, for example, a 3,4-methylenedioxyphenyl group. Aryl groups include, for example, phenyl and naphthyl, including 1-naphthyl and 2-naphthyl. In one aspect, aryl groups are pendant. An example of a pendant ring is a phenyl group substituted with a phenyl group. In one aspect, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, 2,5 morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring which contains from 1 to 4, or in some aspects 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 4, or in some aspects from 1 to 3 or from 1 to 2, heteroatoms selected from N, O, S, B, or P, with remaining ring atoms being carbon. In one aspects, the only heteroatom is nitrogen. In one aspect, the only heteroatom is oxygen. In one aspect, the only heteroatom is sulfur. Monocyclic heteroaryl groups typically have from 5 to 6 ring atoms. In some aspects, bicyclic heteroaryl groups are 8- to 10-membered heteroaryl groups, that is groups containing 8 or 10 ring atoms in which one 5-, 6-, or 7-membered aromatic ring which contains from 1 to 4 heteroatoms selected from N, O, S, B, or P is fused to a second aromatic or non-aromatic ring, wherein the point of attachment is an aromatic ring. When the total number of S and O atoms in the heteroaryl ring exceeds 1, these heteroatoms are not adjacent to one another within the ring. In one aspect, the total number of S and O atoms in the heteroaryl ring is not more than 2. In another aspect, the total number of S and O atoms in the heteroaryl ring is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produced the same counterion. Lists of additional suitable salts may be found, e.g., in Remington's Pharmaceutical Sciences, 17^{th} ed., Mack Publishing Company, Easton, PA., p. 1418 (1985).

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

### Compounds

In one aspect, a compound of Formula I is provided

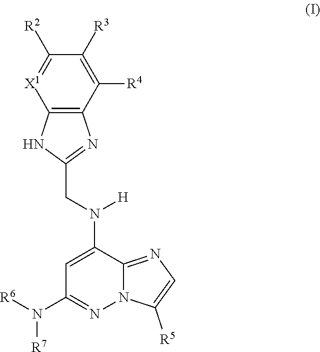

or a pharmaceutically acceptable salt or derivative thereof;
wherein:
X¹ is CR¹ or N;
R¹, R², R³ and R⁴ are independently selected from hydrogen, halo, cyano, azido, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)— (C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, each of which may be optionally substituted with one or more groups selected from Y as allowed by valency;
R⁵ is 5- to 10-membered monocyclic or bicyclic heteroaryl optionally substituted with one or more groups selected from Z as allowed by valency;
R⁶ is selected from C₁-C₆ alkyl and 3- to 8-membered monocyclic or bicyclic heterocycle optionally substituted with one or more groups selected from Z;
R⁷ is selected from hydrogen and C₁-C₆ alkyl;
or R⁶ and R⁷ are brought together with the nitrogen to which they are attached to form a 3- to 8-membered monocyclic or bicyclic heterocycle optionally substituted with one or more groups selected from Z;
Z is independently selected at each occurrence from hydrogen, halo, cyano, azido, oxo, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, each of which may be optionally substituted with one or more groups selected from Y as allowed by valency;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency;
R^{z} is independently selected at each occurrence from hydrogen, halo, C₁-C₆alkyl, C₁-C₆haloalkyl, C₂-C₆halkenyl, C₂-C₆halkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR*, —SR^{x}, and —NR^{x}R^{y}, each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

In some aspects, X¹ is CR¹. In some aspects, X is N.

In some aspects, R¹, R², R³, and R⁴ where present are independently selected from hydrogen, halo, and C₁-C₆ alkyl. In some aspects, R¹, R², R³, and R⁴ where present are independently selected from hydrogen, chloro, fluoro, and methyl.

In some aspects, X¹ is CR¹, R¹ and R⁴ are hydrogen, and R² and R³ are chloro.

In some aspects, X¹ is CR¹, R¹ is chloro and R², R³, and R⁴ are hydrogen.

In some aspects, X¹ is N, and R² and R³ are hydrogen, and R⁴ is methyl.

In some aspects, X¹ is CR¹, R¹, R², and R³ are hydrogen, and R⁴ is fluoro.

In some aspects, X¹ is CR¹. R¹ and R² are hydrogen, and R³ and R⁴ are fluoro.

In some aspects, R⁵ is 5-membered monocyclic heteroaryl having one or two heteroatoms independently selected from N, O, and S and optionally substituted with one or two groups selected from Z as allowed by valency.

In some aspects, R⁵ is selected from pyrazolyl, thienyl, and isoxazolyl optionally substituted with or two groups selected from Z as allowed by valency.

In some aspects, R⁵ is selected from:

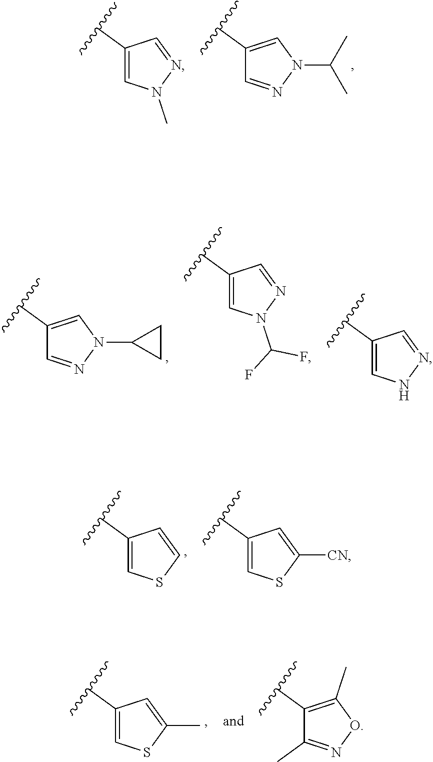

In some aspects, R⁷ is hydrogen, and R⁶ is 5- to 6-membered monocyclic heterocyclic having one or two heteroatoms independently selected from N, O, and S and optionally substituted with one or two groups selected from Z as allowed by valency.

In some aspects, R⁷ is hydrogen and R⁶ is selected from piperazinyl, morpholinyl, or piperidinyl optionally substituted with one or two groups selected from Z as allowed by valency.

In some aspects, R⁶ and R⁷ are brought together with the nitrogen to which they are attached to form a 5- to 6-membered monocyclic heterocycle optionally substituted with one or two groups selected from Z as allowed by valency.

In some aspects, R⁶ and R⁷ are brought together with the nitrogen to which they are attached to form a piperazinyl, morpholinyl, or piperidinyl ring optionally substituted with one or two groups selected from Z as allowed by valency.

In some aspects, —NR⁶R⁷ is selected from:

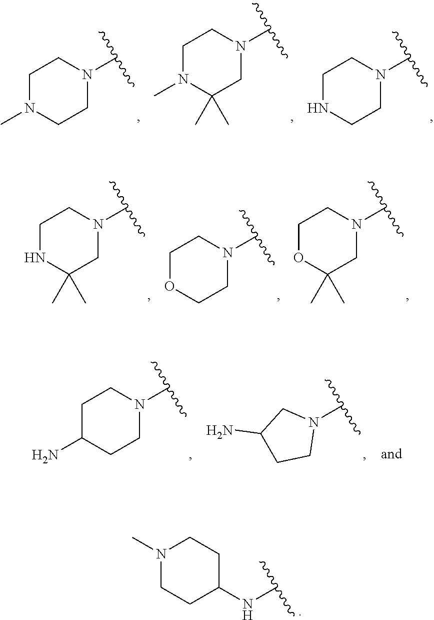

Representative compounds of the present disclosure include, but are not limited to, the compounds found in Table 1 below:

TABLE 1
Representative Compounds of Formula I

| | Cmpd | | |
|---|---|---|---|
| Structure | # | Name | MW |
| 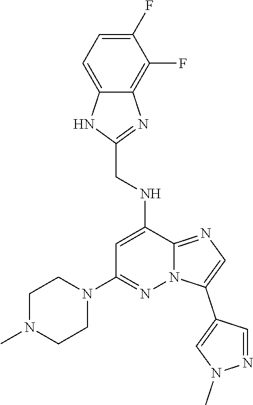 [SMILES: CN1CCN(c2cc(NCc3nc4c(F)c(F)ccc4[nH]3)c3ncc(-c4cnn(C)c4)n3n2)CC1] | 5 | N-((4,5-difluoro-1H-benzo[d]imidazol-2- yl)methyl)-3-(1-methyl-1H-pyrazol-4-yl)-6-(4- methylpiperazin-1-yl)imidazo[1,2-b]pyridazin- 8-amine | 478.51 |
| 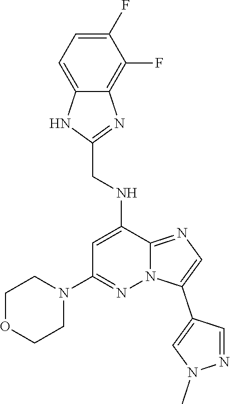 [SMILES: Cn1cc(-c2cnc3c(NCc4nc5c(F)c(F)ccc5[nH]4)cc(N4CCOCC4)nn23)cn1] | 6 | N-((4,5-difluoro-1H-benzo[d]imidazol-2- yl)methyl)-3-(1-methyl-1H-pyrazol-4-yl)-6- morpholinoimidazo[1,2-b]pyridazin-8-amine | 466.47 |
| 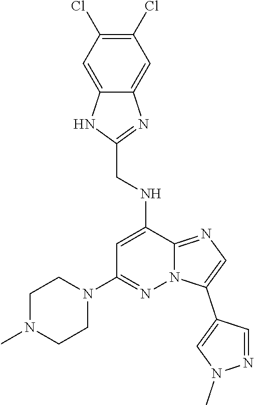 [SMILES: CN1CCN(c2cc(NCc3nc4cc(Cl)c(Cl)cc4[nH]3)c3ncc(-c4cnn(C)c4)n3n2)CC1] | 8 | N-((5,6-dichloro-1H-benzo[d]imidazol-2- yl)methyl)-3-(1-methyl-1H-pyrazol-4-yl)-6-(4- methylpiperazin-1-yl)imidazo[1,2-b]pyridazin- 8-amine | 511.42 |
| 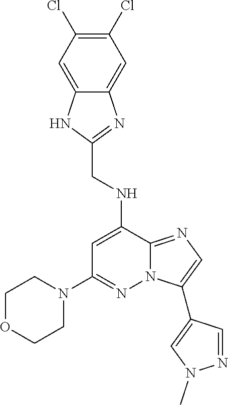 [SMILES: Cn1cc(-c2cnc3c(NCc4nc5cc(Cl)c(Cl)cc5[nH]4)cc(N4CCOCC4)nn23)cn1] | 9 | N-((5,6-dichloro-1H-benzo[d]imidazol-2- yl)methyl)-3-(1-methyl-1H-pyrazol-4-yl)-6- morpholinoimidazo[1,2-b]pyridazin-8-amine | 498.37 |
| 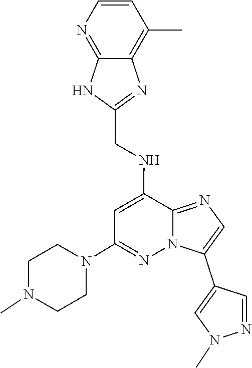 [SMILES: Cc1ccnc2[nH]c(CNc3cc(N4CCN(C)CC4)nn4c(-c5cnn(C)c5)cnc34)nc12] | 11 | 3-(1-methyl-1H-pyrazol-4-yl)-N-((7-methyl- 3H-imidazo[4,5-b]pyridin-2-yl)methyl)-6-(4- methylpiperazin-1-yl)imidazo[1,2-b]pyridazin- 8-amine | 457.55 |
| 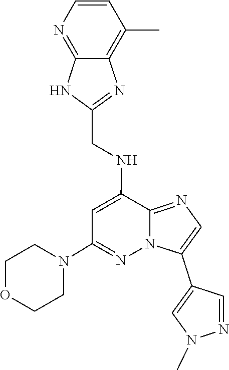 [SMILES: Cc1ccnc2[nH]c(CNc3cc(N4CCOCC4)nn4c(-c5cnn(C)c5)cnc34)nc12] | 12 | 3-(1-methyl-1H-pyrazol-4-yl)-N-((7-methyl- 3H-imidazo[4,5-b]pyridin-2-yl)methyl)-6- morpholinoimidazo[1,2-b]pyridazin-8-amine | 444.50 |
| 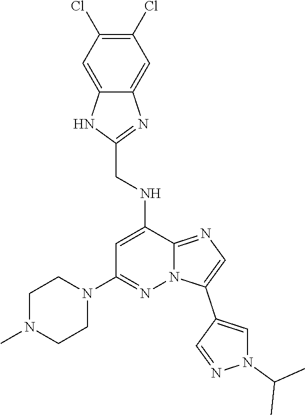 [SMILES: CC(C)n1cc(-c2cnc3c(NCc4nc5cc(Cl)c(Cl)cc5[nH]4)cc(N4CCN(C)CC4)nn23)cn1] | 14 | N-((5,6-dichloro-1H-benzo[d]imidazol-2- yl)methyl)-3-(1-isopropyl-1H-pyrazol-4-yl)-6- (4-methylpiperazin-1-yl)imidazo[1,2- b]pyridazin-8-amine | 539.47 |
| 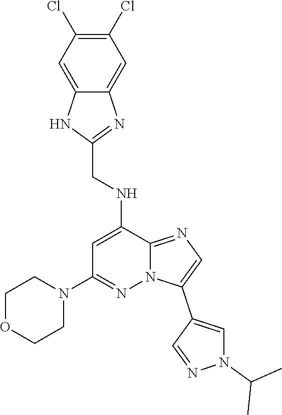 [SMILES: CC(C)n1cc(-c2cnc3c(NCc4nc5cc(Cl)c(Cl)cc5[nH]4)cc(N4CCOCC4)nn23)cn1] | 15 | N-((5,6-dichloro-1H-benzo[d]imidazol-2- yl)methyl)-3-(1-isopropyl-1H-pyrazol-4-yl)-6- morpholinoimidazo[1,2-b]pyridazin-8-aminc | 526.43 |
| 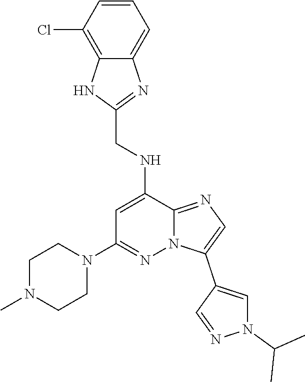 [SMILES: CC(C)n1cc(-c2cnc3c(NCc4nc5cccc(Cl)c5[nH]4)cc(N4CCN(C)CC4)nn23)cn1] | 17 | N-((7-chloro-1H-benzo[d]imidazol-2- yl)methyl)-3-(1-isopropyl-1H-pyrazol-4-yl)-6- (4-methylpiperazin-1-yl)imidazo[1,2- b]pyridazin-8-amine | 505.03 |
| 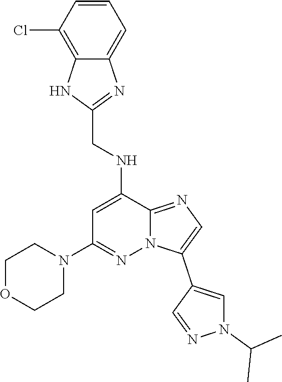 [SMILES: CC(C)n1cc(-c2cnc3c(NCc4nc5cccc(Cl)c5[nH]4)cc(N4CCOCC4)nn23)cn1] | 18 | N-((7-chloro-1H-benzo[d]imidazol-2- yl)methyl)-3-(1-isopropyl-1H-pyrazol-4-yl)-6- morpholinoimidazo[1,2-b]pyridazin-8-amine | 491.98 |
| 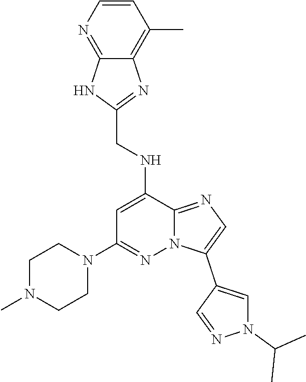 [SMILES: Cc1ccnc2[nH]c(CNc3cc(N4CCN(C)CC4)nn4c(-c5cnn(C(C)C)c5)cnc34)nc12] | 20 | 3-(1-isopropyl-1H-pyrazol-4-yl)-N-((7-methyl- 3H-imidazo[4,5-b]pyridin-2-yl)methyl)-6-(4- methylpiperazin-1-yl)imidazo[1,2-b]pyridazin- 8-amine | 485.60 |
| 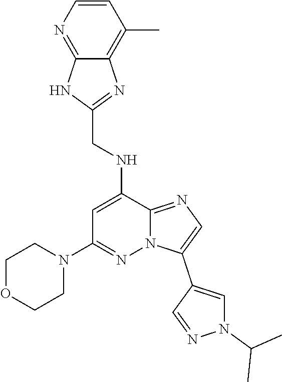 [SMILES: Cc1ccnc2[nH]c(CNc3cc(N4CCOCC4)nn4c(-c5cnn(C(C)C)c5)cnc34)nc12] | 21 | 3-(1-isopropyl-1H-pyrazol-4-yl)-N-((7-methyl- 3H-imidazo[4,5-b]pyridin-2-yl)methyl)-6- morpholinoimidazo[1,2-b]pyridazin-8-amine | 472.56 |
| 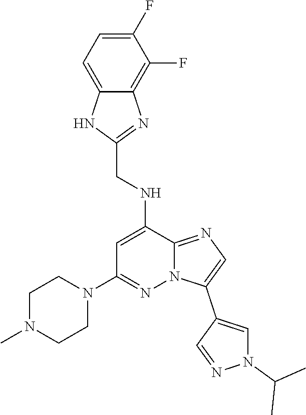 [SMILES: CC(C)n1cc(-c2cnc3c(NCc4nc5c(F)c(F)ccc5[nH]4)cc(N4CCN(C)CC4)nn23)cn1] | 23 | N-((4,5-difluoro-1H-benzo[d]imidazol-2- yl)methyl)-3-(1-isopropyl-1H-pyrazol-4-yl)-6- (4-methylpiperazin-1-yl)imidazo[1,2- b]pyridazin-8-amine | 506.57 |
| 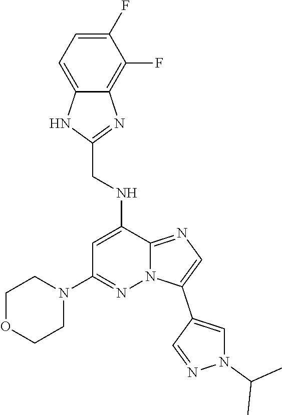 [SMILES: CC(C)n1cc(-c2cnc3c(NCc4nc5c(F)c(F)ccc5[nH]4)cc(N4CCOCC4)nn23)cn1] | 24 | N-((4,5-difluoro-1H-benzo[d]imidazol-2- yl)methyl)-3-(1-isopropyl-1H-pyrazol-4-yl)-6- morpholinoimidazo[1,2-b]pyridazin-8-amine | 493.52 |
| 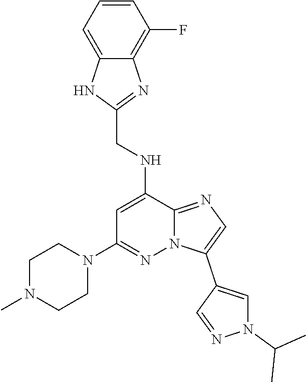 [SMILES: CC(C)n1cc(-c2cnc3c(NCc4nc5c(F)cccc5[nH]4)cc(N4CCN(C)CC4)nn23)cn1] | 26 | N-((4-fluoro-1H-benzo[d]imidazol-2- yl)methyl)-3-(1-isopropyl-1H-pyrazol-4-yl)-6- (4-methylpiperazin-1-yl)imidazo[1,2- b]pyridazin-8-amine | 488.58 |
| 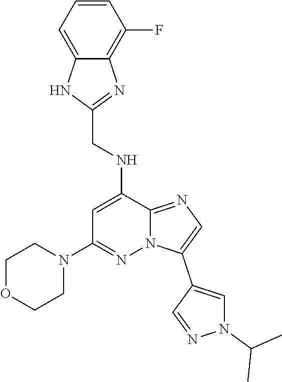 [SMILES: CC(C)n1cc(-c2cnc3c(NCc4nc5c(F)cccc5[nH]4)cc(N4CCOCC4)nn23)cn1] | 27 | N-((4-fluoro-1H-benzo[d]imidazol-2- yl)methyl)-3-(1-isopropyl-1H-pyrazol-4-yl)-6- morpholinoimidazo[1,2-b]pyridazin-8-amine | 475.53 |
| 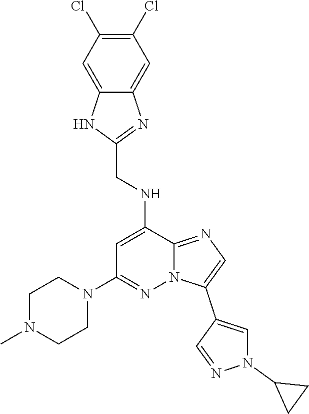 [SMILES: CN1CCN(c2cc(NCc3nc4cc(Cl)c(Cl)cc4[nH]3)c3ncc(-c4cnn(C5CC5)c4)n3n2)CC1] | 29 | 3-(1-cyclopropyl-1H-pyrazol-4-yl)-N-((5,6- dichloro-1H-benzo[d]imidazol-2-yl)methyl)-6- (4-methylpiperazin-1-yl)imidazo[1,2- b]pyridazin-8-amine | 537.45 |
| 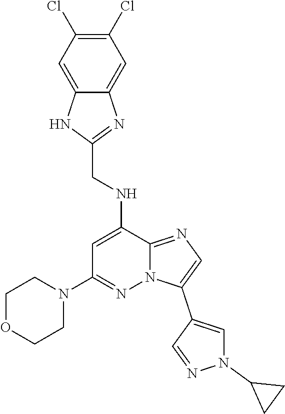 [SMILES: Clc1cc2nc(CNc3cc(N4CCOCC4)nn4c(-c5cnn(C6CC6)c5)cnc34)[nH]c2cc1Cl] | 30 | 3-(1-cyclopropyl-1H-pyrazol-4-yl)-N-((5,6- dichloro-1H-benzo[d]imidazol-2-yl)methyl)-6- morpholinoimidazo[1,2-b]pyridazin-8-amine | 524.41 |
| 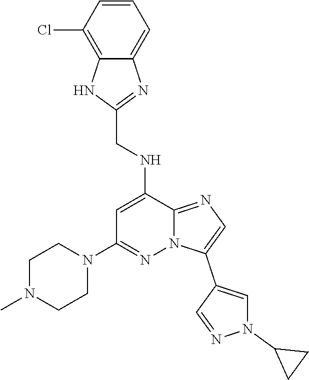 [SMILES: CN1CCN(c2cc(NCc3nc4cccc(Cl)c4[nH]3)c3ncc(-c4cnn(C5CC5)c4)n3n2)CC1] | 32 | N-((7-chloro-1H-benzo[d]imidazol-2- yl)methyl)-3-(1-cyclopropyl-1H-pyrazol-4-yl)- 6-(4-methylpiperazin-1-yl)imidazo[1,2- b]pyridazin-8-amine | 503.01 |
| 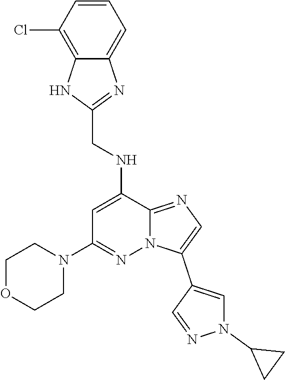 [SMILES: Clc1cccc2nc(CNc3cc(N4CCOCC4)nn4c(-c5cnn(C6CC6)c5)cnc34)[nH]c12] | 33 | N-((7-chloro-1H-benzo[d]imidazol-2- yl)methyl)-3-(1-cyclopropyl-1H-pyrazol-4-yl)- 6-morpholinoimidazo[1,2-b]pyridazin-8-amine | 489.97 |
| 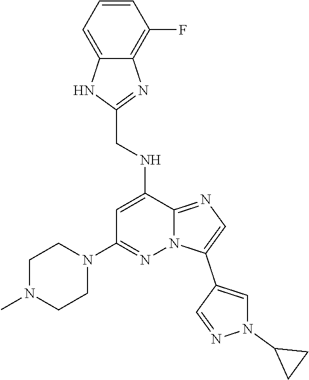 [SMILES: CN1CCN(c2cc(NCc3nc4c(F)cccc4[nH]3)c3ncc(-c4cnn(C5CC5)c4)n3n2)CC1] | 35 | 3-(1-cyclopropyl-1H-pyrazol-4-yl)-N-((4- fluoro-1H-benzo[d]imidazol-2-yl)methyl)-6-(4- methylpiperazin-1-yl)imidazo[1,2-b]pyridazin- 8-amine | 486.56 |
| 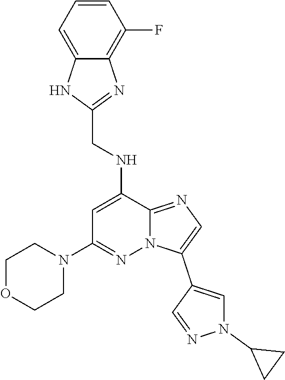 [SMILES: Fc1cccc2[nH]c(CNc3cc(N4CCOCC4)nn4c(-c5cnn(C6CC6)c5)cnc34)nc12] | 36 | 3-(1-cyclopropyl-1H-pyrazol-4-yl)-N-((4- fluoro-1H-benzo[d]imidazol-2-yl)methyl)-6- morpholinoimidazo[1,2-b]pyridazin-8-amine | 473.52 |
| 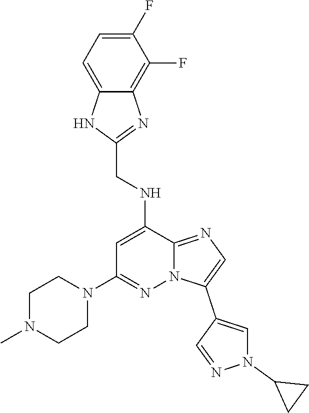 [SMILES: CN1CCN(c2cc(NCc3nc4c(F)c(F)ccc4[nH]3)c3ncc(-c4cnn(C5CC5)c4)n3n2)CC1] | 38 | 3-(1-cyclopropyl-1H-pyrazol-4-yl)-N-((4,5- difluoro-1H-benzo[d]imidazol-2-yl)methyl)-6- (4-methylpiperazin-1-yl)imidazo[1,2- b]pyridazin-8-amine | 504.55 |
| 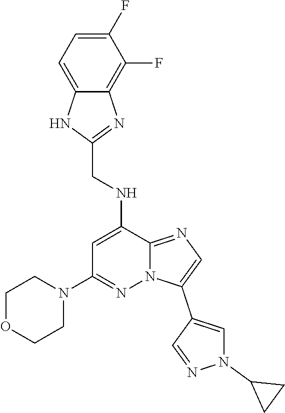 [SMILES: Fc1ccc2[nH]c(CNc3cc(N4CCOCC4)nn4c(-c5cnn(C6CC6)c5)cnc34)nc2c1F] | 39 | 3-(1-cyclopropyl-1H-pyrazol-4-yl)-N-((4,5- difluoro-1H-benzo[d]imidazol-2-yl)mcthyl)-6- morpholinoimidazo[1,2-b]pyridazin-8-amine | 491.51 |
| 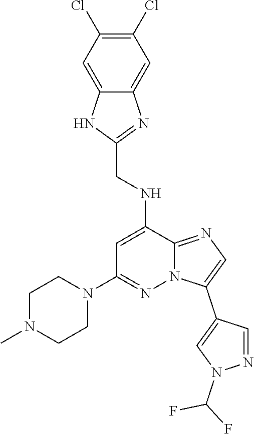 [SMILES: CN1CCN(c2cc(NCc3nc4cc(Cl)c(Cl)cc4[nH]3)c3ncc(-c4cnn(C(F)F)c4)n3n2)CC1] | 41 | N-((5,6-dichloro-1H-benzo[d]imidazol-2- yl)methyl)-3-(1-(difluoromethyl)-1H-pyrazol- 4-yl)-6-(4-methylpiperazin-1-yl)imidazo[1,2- b]pyridazin-8-amine | 547.40 |
| 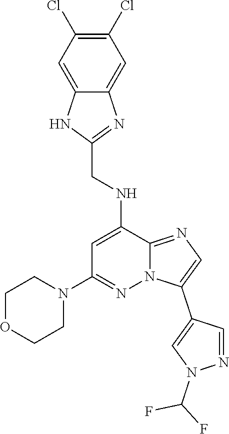 [SMILES: FC(F)n1cc(-c2cnc3c(NCc4nc5cc(Cl)c(Cl)cc5[nH]4)cc(N4CCOCC4)nn23)cn1] | 42 | N-((5,6-dichloro-1H-benzo[d]imidazol-2- yl)methyl)-3-(1-(difluoromethyl)-1H-pyrazol- 4-yl)-6-morpholinoimidazo[1,2-b]pyridazin-8- amine | 534.35 |
| 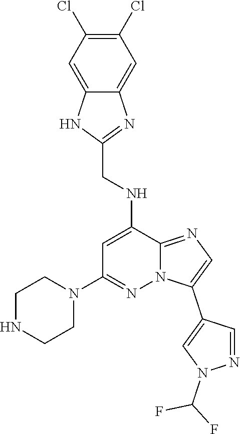 [SMILES: FC(F)n1cc(-c2cnc3c(NCc4nc5cc(Cl)c(Cl)cc5[nH]4)cc(N4CCNCC4)nn23)cn1] | 43 | N-((5,6-dichloro-1H-benzo[d]imidazol-2- yl)methyl)-3-(1-(difluoromethyl)-1H-pyrazol- 4-yl)-6-(piperazin-1-yl)imidazo[1,2- b]pyridazin-8-amine | 533.37 |
| 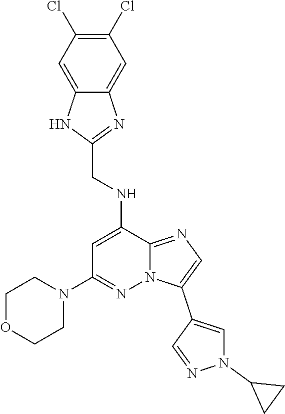 [SMILES: Clc1cc2nc(CNc3cc(N4CCOCC4)nn4c(-c5cnn(C6CC6)c5)cnc34)[nH]c2cc1Cl] | 44 | 6-(3-aminopyrrolidin-1-yl)-N-((5,6-dichloro- 1H-benzo[d]imidazol-2-yl)methyl)-3-(1- (difluoromethyl)-1H-pyrazol-4-yl)imidazo[1,2- b]pyridazin-8-amine | 533.37 |
| 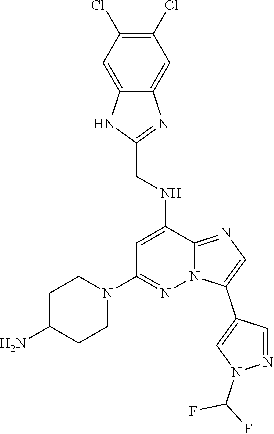 [SMILES: NC1CCN(c2cc(NCc3nc4cc(Cl)c(Cl)cc4[nH]3)c3ncc(-c4cnn(C(F)F)c4)n3n2)CC1] | 45 | 6-(4-aminopiperidin-1-yl)-N-((5,6-dichloro- 1H-benzo[d]imidazol-2-yl)methyl)-3-(1- (difluoromethyl)-1H-pyrazol-4-yl)imidazo[1,2- b]pyridazin-8-amine | 547.40 |
| 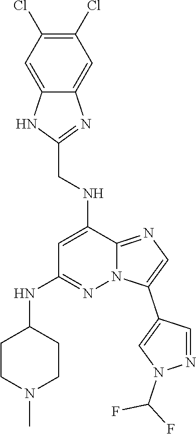 [SMILES: CN1CCC(Nc2cc(NCc3nc4cc(Cl)c(Cl)cc4[nH]3)c3ncc(-c4cnn(C(F)F)c4)n3n2)CC1] | 46 | N⁸-((5,6-dichloro-1H-benzo[d]imidazol-2- yl)methyl)-3-(1-(difluoromethyl)-1H-pyrazol- 4-yl)-N⁶-(1-methylpiperidin-4-yl)imidazo[1,2- b]pyridazine-6,8-diamine | 561.42 |
| 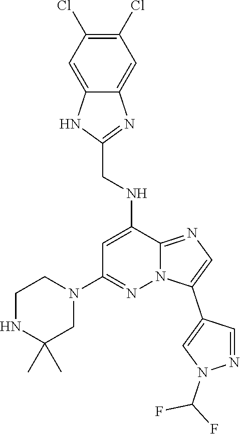 [SMILES: CC1(C)CN(c2cc(NCc3nc4cc(Cl)c(Cl)cc4[nH]3)c3ncc(-c4cnn(C(F)F)c4)n3n2)CCN1] | 47 | N-((5,6-dichloro-1H-benzo[d]imidazol-2- yl)methyl)-3-(1-(difluoromethyl)-1H-pyrazol- 4-yl)-6-(3,3-dimethylpiperazin-1- yl)imidazo[1,2-b]pyridazin-8-amine | 561.42 |
| 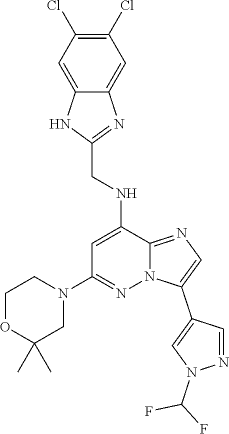 [SMILES: CC1(C)CN(c2cc(NCc3nc4cc(Cl)c(Cl)cc4[nH]3)c3ncc(-c4cnn(C(F)F)c4)n3n2)CCO1] | 48 | N-((5,6-dichloro-1H-benzo[d]imidazol-2- yl)methyl)-3-(1-(difluoromethyl)-1H-pyrazol- 4-yl)-6-(2,2-dimethylmorpholino)imidazo[1,2- b]pyridazin-8-amine | 562.41 |
| 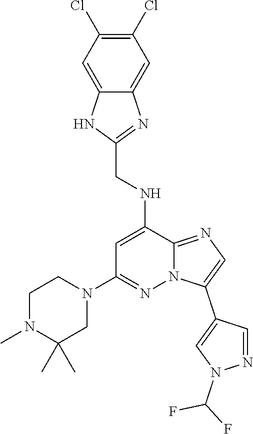 [SMILES: CN1CCN(c2cc(NCc3nc4cc(Cl)c(Cl)cc4[nH]3)c3ncc(-c4cnn(C(F)F)c4)n3n2)CC1(C)C] | 49 | N-((5,6-dichloro-1H-benzo[d]imidazol-2- yl)methyl)-3-(1-(difluoromethyl)-1H-pyrazol- 4-yl)-6-(3,3,4-trimethylpiperazin-1- yl)imidazo[1,2-b]pyridazin-8-amine | 575.45 |
| 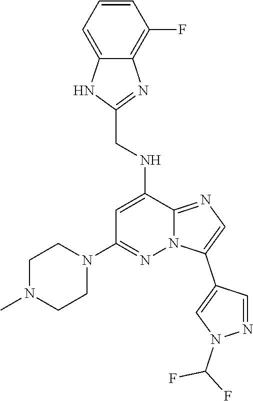 [SMILES: CN1CCN(c2cc(NCc3nc4c(F)cccc4[nH]3)c3ncc(-c4cnn(C(F)F)c4)n3n2)CC1] | 51 | 3-(1-(difluoromethyl)-1H-pyrazol-4-yl)-N-((4- fluoro-1H-benzo[d]imidazol-2-yl)methyl)-6-(4- methylpiperazin-1-yl)imidazo[1,2-b]pyridazin- 8-amine | 496.50 |
| 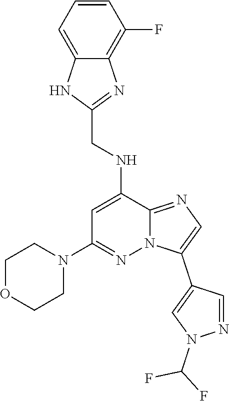 [SMILES: Fc1cccc2[nH]c(CNc3cc(N4CCOCC4)nn4c(-c5cnn(C(F)F)c5)cnc34)nc12] | 52 | 3-(1-(difluoromethyl)-1H-pyrazol-4-yl)-N-((4- fluoro-1H-benzo[d]imidazol-2-yl)methyl)-6- morpholinoimidazo[1,2-b]pyridazin-8-amine | 483.46 |
| 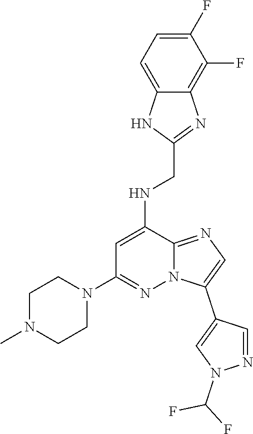 [SMILES: CN1CCN(c2cc(NCc3nc4c(F)c(F)ccc4[nH]3)c3ncc(-c4cnn(C(F)F)c4)n3n2)CC1] | 54 | N-((4,5-difluoro-1H-benzo[d]imidazol-2- yl)methyl)-3-(1-(difluoromethyl)-1H-pyrazol- 4-yl)-6-(4-methylpiperazin-1-yl)imidazo[1,2- b]pyridazin-8-amine | 514.49 |
| 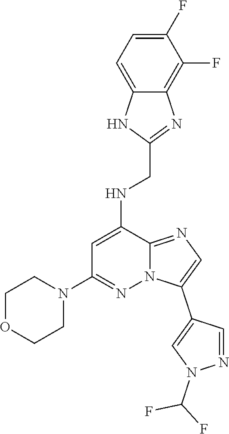 [SMILES: Fc1ccc2[nH]c(CNc3cc(N4CCOCC4)nn4c(-c5cnn(C(F)F)c5)cnc34)nc2c1F] | 55 | N-((4,5-difluoro-1H-benzo[d]imidazol-2- yl)methyl)-3-(1-(difluoromethyl)-1H-pyrazol- 4-yl)-6-morpholinoimidazo[1,2-b]pyridazin-8- amine | 501.45 |
| 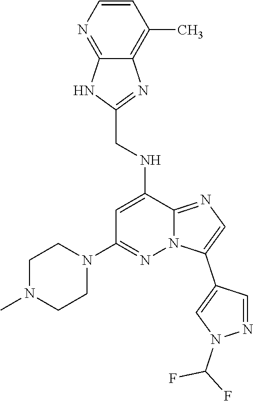 [SMILES: Cc1ccnc2[nH]c(CNc3cc(N4CCN(C)CC4)nn4c(-c5cnn(C(F)F)c5)cnc34)nc12] | 57 | 3-(1-(difluoromethyl)-1H-pyrazol-4-yl)-N-((7- methyl-3H-imidazo[4,5-b]pyridin-2- yl)methyl)-6-(4-methylpiperazin-1- yl)imidazo[1,2-b]pyridazin-8-amine | 493.53 |
| 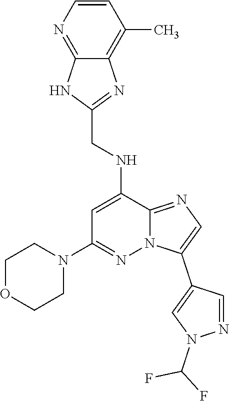 [SMILES: Cc1ccnc2[nH]c(CNc3cc(N4CCOCC4)nn4c(-c5cnn(C(F)F)c5)cnc34)nc12] | 58 | 3-(1-(difluoromethyl)-1H-pyrazol-4-yl)-N-((7- methyl-3H-imidazo[4,5-b]pyridin-2- yl)methyl)-6-morpholinoimidazo[1,2- b]pyridazin-8-amine | 480.48 |
| 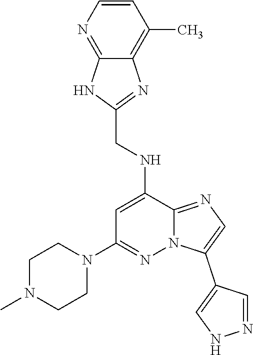 [SMILES: Cc1ccnc2[nH]c(CNc3cc(N4CCN(C)CC4)nn4c(-c5cn[nH]c5)cnc34)nc12] | 60 | N-((5,6-dichloro-1H-benzo[d]imidazol-2- yl)methyl)-6-(4-methylpiperazin-1-yl)-3-(1H- pyrazol-4-yl)imidazo[1,2-b]pyridazin-8-amine | 497.39 |
| 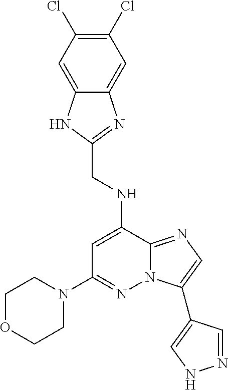 [SMILES: Clc1cc2nc(CNc3cc(N4CCOCC4)nn4c(-c5cn[nH]c5)cnc34)[nH]c2cc1Cl] | 61 | N-((5,6-dichloro-1H-benzo[d]imidazol-2- yl)methyl)-6-morpholino-3-(1H-pyrazol-4- yl)imidazo[1,2-b]pyridazin-8-amine | 484.35 |
| 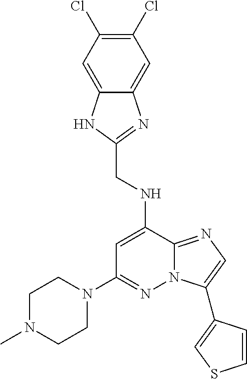 [SMILES: CN1CCN(c2cc(NCc3nc4cc(Cl)c(Cl)cc4[nH]3)c3ncc(-c4ccsc4)n3n2)CC1] | 63 | N-((5,6-dichloro-1H-benzo[d]imidazol-2- (yl)methyl)-6-(4-methylpiperazin-1-yl)-3- (thiophen-3-yl)imidazo[1,2-b]pyridazin-8- aminc | 513.45 |
| 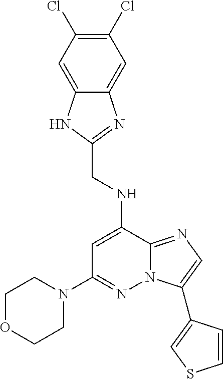 [SMILES: Clc1cc2nc(CNc3cc(N4CCOCC4)nn4c(-c5ccsc5)cnc34)[nH]c2cc1Cl] | 64 | N-((5,6-dichloro-1H-benzo[d]imidazol-2- yl)methyl)-6-morpholino-3-(thiophen-3- yl)imidazo[1,2-b]pyridazin-8-amine | 500.40 |
| 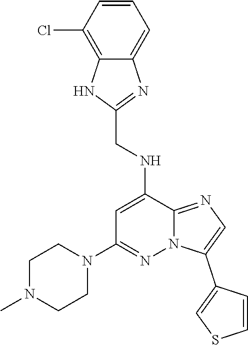 [SMILES: CN1CCN(c2cc(NCc3nc4cccc(Cl)c4[nH]3)c3ncc(-c4ccsc4)n3n2)CC1] | 66 | N-((7-chloro-1H-benzo[d]imidazol-2- yl)methyl)-6-(4-methylpiperazin-1-yl)-3- (thiophen-3-yl)imidazo[1,2-b]pyridazin-8- amine | 479.00 |
| 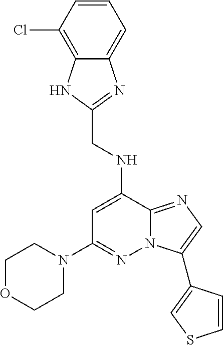 [SMILES: Clc1cccc2nc(CNc3cc(N4CCOCC4)nn4c(-c5ccsc5)cnc34)[nH]c12] | 67 | N-((7-chloro-1H-benzo[d]imidazol-2- yl)methyl)-6-morpholino-3-(thiophen-3- yl)imidazo[1,2-b]pyridazin-8-amine | 465.96 |
| 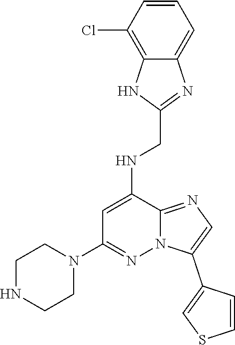 [SMILES: Clc1cccc2nc(CNc3cc(N4CCNCC4)nn4c(-c5ccsc5)cnc34)[nH]c12] | 68 | N-((7-chloro-1H-benzo[d]imidazol-2- yl)methyl)-6-(piperazin-1-yl)-3-(thiophen-3- yl)imidazo[1,2-b]pyridazin-8-amine | 464.98 |
| 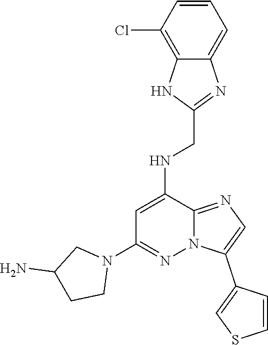 [SMILES: NC1CCN(c2cc(NCc3nc4cccc(Cl)c4[nH]3)c3ncc(-c4ccsc4)n3n2)C1] | 69 | 6-(3-aminopyrrolidin-1-yl)-N-((7-chloro-1H- benzo[d]imidazol-2-yl)methyl)-3-(thiophen-3- yl)imidazo[1,2-b]pyridazin-8-amine | 464.98 |
| 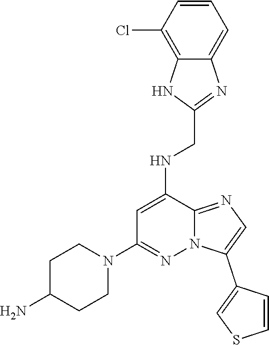 [SMILES: NC1CCN(c2cc(NCc3nc4cccc(Cl)c4[nH]3)c3ncc(-c4ccsc4)n3n2)CC1] | 70 | 6-(4-aminopiperidin-1-yl)-N-((7-chloro-1H- benzo[d]imidazol-2-yl)methyl)-3-(thiophen-3- yl)imidazo[1,2-b]pyridazin-8-amine | 479.00 |
| 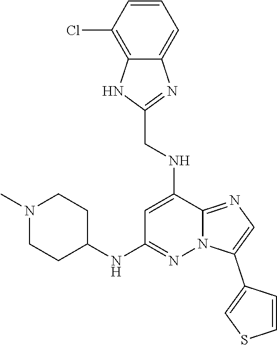 [SMILES: CN1CCC(Nc2cc(NCc3nc4cccc(Cl)c4[nH]3)c3ncc(-c4ccsc4)n3n2)CC1] | 71 | N⁸-((7-chloro-1H-benzo[d]imidazol-2- yl)methyl)-N⁶-(1-methylpiperidin-4-yl)-3- (thiophen-3-yl)imidazo[1,2-b]pyridazine-6,8- diamine | 493.03 |
| 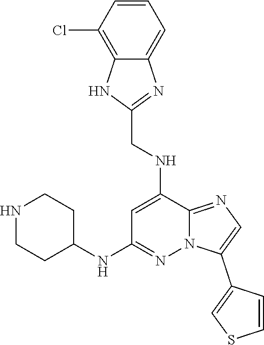 [SMILES: Clc1cccc2nc(CNc3cc(NC4CCNCC4)nn4c(-c5ccsc5)cnc34)[nH]c12] | 72 | N⁸-((7-chloro-1H-benzo[d]imidazol-2- yl)methyl)-N⁶-(piperidin-4-yl)-3-(thiophen-3- yl)imidazo[1,2-b]pyridazine-6,8-diamine | 479.00 |
| 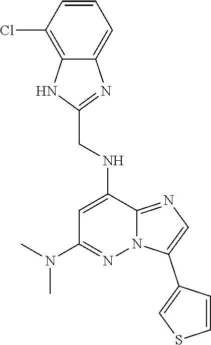 [SMILES: CN(C)c1cc(NCc2nc3cccc(Cl)c3[nH]2)c2ncc(-c3ccsc3)n2n1] | 73 | N⁸-((7-chloro-1H-benzo[d]imidazol-2- yl)methyl)-N⁶,N⁶-dimethyl-3-(thiophen-3- yl)imidazo[1,2-b]pyridazine-6,8-diamine | 423.92 |
| 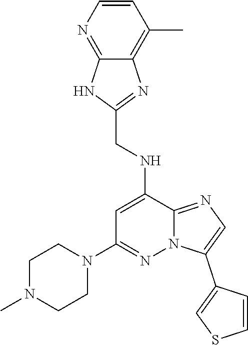 [SMILES: Cc1ccnc2[nH]c(CNc3cc(N4CCN(C)CC4)nn4c(-c5ccsc5)cnc34)nc12] | 75 | N-((7-methyl-3H-imidazo[4,5-b]pyridin-2- yl)methyl)-6-(4-methylpiperazin-1-yl)-3- (thiophen-3-yl)imidazo[1,2-b]pyridazin-8- amine | 459.58 |
| 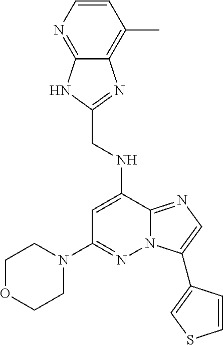 [SMILES: Cc1ccnc2[nH]c(CNc3cc(N4CCOCC4)nn4c(-c5ccsc5)cnc34)nc12] | 76 | N-((7-methyl-3H-imidazo[4,5-b]pyridin-2- yl)methyl)-6-morpholino-3-(thiophen-3- yl)imidazo[1,2-b]pyridazin-8-amine | 446.53 |
| 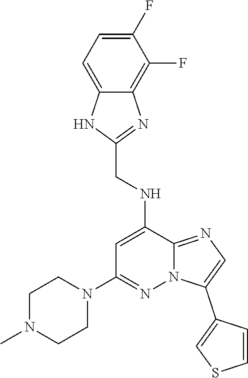 [SMILES: CN1CCN(c2cc(NCc3nc4c(F)c(F)ccc4[nH]3)c3ncc(-c4ccsc4)n3n2)CC1] | 78 | N-((4,5-difluoro-1H-benzo[d]imidazol-2- yl)methyl)-6-(4-methylpiperazin-1-yl)-3- (thiophen-3-yl)imidazo[1,2-b]pyridazin-8- amine | 480.54 |
| 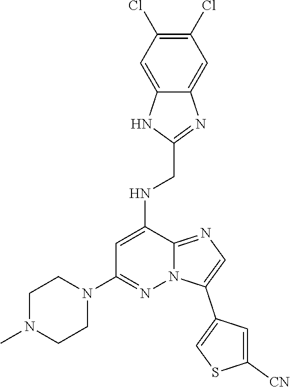 [SMILES: CN1CCN(c2cc(NCc3nc4cc(Cl)c(Cl)cc4[nH]3)c3ncc(-c4csc(C#N)c4)n3n2)CC1] | 79 | 4-(8-(((5,6-dichloro-1H-benzo[d]imidazol-2- yl)methyl)amino)-6-(4-methylpiperazin-1- yl)imidazo[1,2-b]pyridazin-3-yl)thiophene-2- carbonitrile | 538.46 |
| 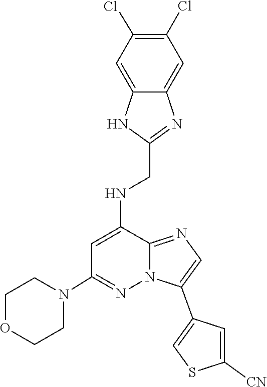 [SMILES: N#Cc1cc(-c2cnc3c(NCc4nc5cc(Cl)c(Cl)cc5[nH]4)cc(N4CCOCC4)nn23)cs1] | 80 | 4-(8-(((5,6-dichloro-1H-benzo[d]imidazol-2- yl)methyl)amino)-6-morpholinoimidazo[1,2- b]pyridazin-3-yl)thiophene-2-carbonitrile | 525.41 |
| 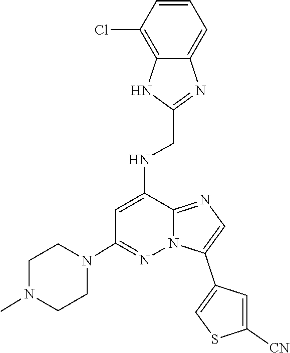 [SMILES: CN1CCN(c2cc(NCc3nc4cccc(Cl)c4[nH]3)c3ncc(-c4csc(C#N)c4)n3n2)CC1] | 81 | 4-(8-(((7-chloro-1H-benzo[d]imidazol-2- yl)methyl)amino)-6-(4-methylpiperazin-1- yl)imidazo[1,2-b]pyridazin-3-yl)thiophene-2- carbonitrile | 504.01 |
| 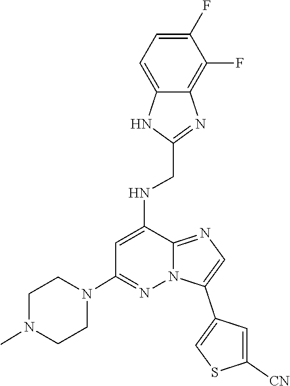 [SMILES: CN1CCN(c2cc(NCc3nc4c(F)c(F)ccc4[nH]3)c3ncc(-c4csc(C#N)c4)n3n2)CC1] | 82 | 4-(8-(((4,5-difluoro-1H-benzo[d]imidazol-2- yl)methyl)amino)-6-(4-methylpiperazin-1- yl)imidazo[1,2-b]pyridazin-3-yl)thiophene-2- carbonitrile | 505.55 |
| 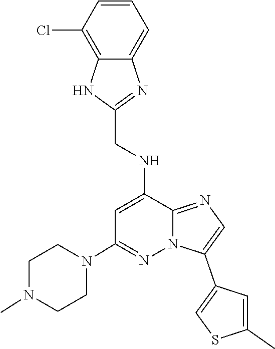 | 84 | N-((7-chloro-1H-benzo[d]imidazol-2- yl)methyl)-6-(4-methylpiperazin-1-yl)-3-(5- methylthiophen-3-yl)imidazo[1,2-b]pyridazin- 8-amine | 492.03 |
| 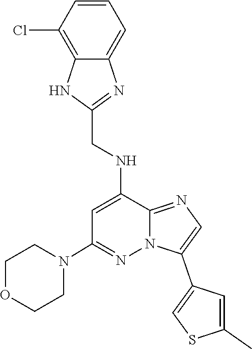 [SMILES: Cc1cc(-c2cnc3c(NCc4nc5cccc(Cl)c5[nH]4)cc(N4CCOCC4)nn23)cs1] | 85 | N-((7-chloro-1H-benzo[d]imidazol-2- yl)methyl)-3-(5-methylthiophen-3-yl)-6- morpholinoimidazo[1,2-b]pyridazin-8-amine | 479.99 |
| 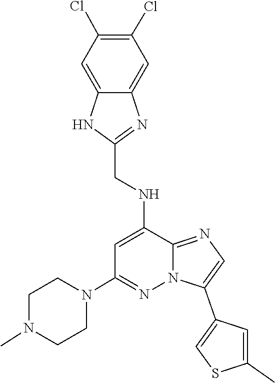 | 87 | N-((5,6-dichloro-1H-benzo[d]imidazol-2- yl)methyl)-6-(4-methylpiperazin-1-yl)-3-(5- methylthiophen-3-yl)imidazo[1,2-b]pyridazin- 8-amine | 527.47 |
| 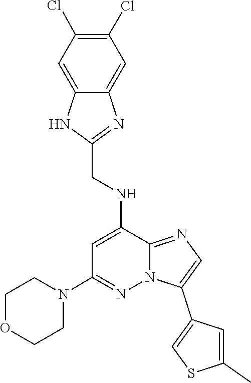 [SMILES: Cc1cc(-c2cnc3c(NCc4nc5cc(Cl)c(Cl)cc5[nH]4)cc(N4CCOCC4)nn23)cs1] | 88 | N-((5,6-dichloro-1H-benzo[d]imidazol-2- yl)methyl)-3-(5-methylthiophen-3-yl)-6- morpholinoimidazo[1,2-b]pyridazin-8-amine | 514.43 |
| 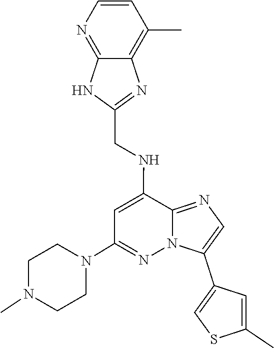 | 90 | N-((7-methyl-3H-imidazo[4,5-b]pyridin-2- yl)methyl)-6-(4-methylpiperazin-1-yl)-3-(5- methylthiophen-3-yl)imidazo[1,2-b]pyridazin- 8-amine | 473.60 |
| 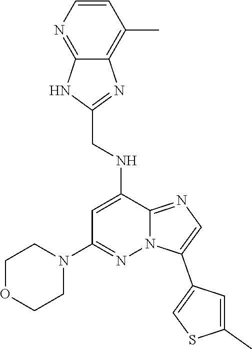 [SMILES: Cc1cc(-c2cnc3c(NCc4nc5c(C)ccnc5[nH]4)cc(N4CCOCC4)nn23)cs1] | 91 | N-((7-methyl-3H-imidazo[4,5-b]pyridin-2- yl)methyl)-3-(5-methylthiophen-3-yl)-6- morpholinoimidazo[1,2-b]pyridazin-8-amine | 460.56 |
| 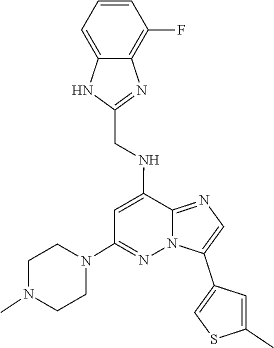 | 93 | N-((4-fluoro-1H-benzo[d]imidazol-2- yl)methyl)-6-(4-methylpiperazin-1-yl)-3-(5- methylthiophen-3-yl)imidazo[1,2-b]pyridazin- 8-amine | 476.58 |
| 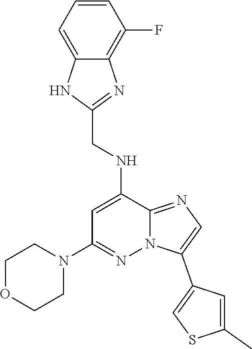 [SMILES: Cc1cc(-c2cnc3c(NCc4nc5c(F)cccc5[nH]4)cc(N4CCOCC4)nn23)cs1] | 94 | N-((4-fluoro-1H-benzo[d]imidazol-2- yl)methyl)-3-(5-methylthiophen-3-yl)-6- morpholinoimidazo[1,2-b]pyridazin-8-amine | 463.54 |
| 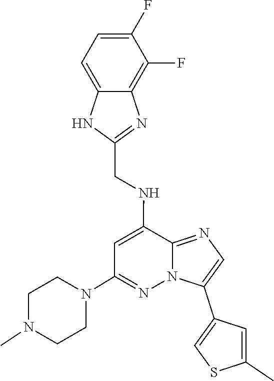 | 96 | N-((4,5-difluoro-1H-benzo[d]imidazol-2- yl)methyl)-6-(4-methylpiperazin-1-yl)-3-(5- methylthiophen-3-yl)imidazo[1,2-b]pyridazin- 8-amine | 494.57 |
| 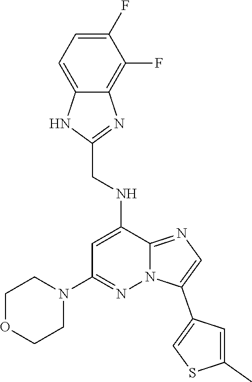 [SMILES: Cc1cc(-c2cnc3c(NCc4nc5c(F)c(F)ccc5[nH]4)cc(N4CCOCC4)nn23)cs1] | 97 | N-((4,5-difluoro-1H-benzo[d]imidazol-2- yl)methyl)-3-(5-methylthiophen-3-yl)-6- morpholinoimidazo[1,2-b]pyridazin-8-amine | 481.53 |
| 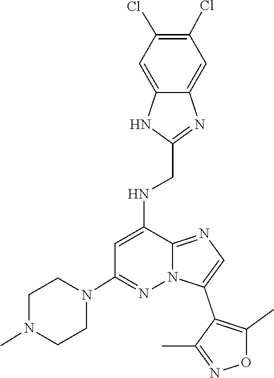 | 99 | N-((5,6-dichloro-1H-benzo[d]imidazol-2- yl)methyl)-3-(3,5-dimethylisoxazol-4-yl)-6-(4- methylpiperazin-1-yl)imidazo[1,2-b]pyridazin- 8-amine | 526.43 |
| 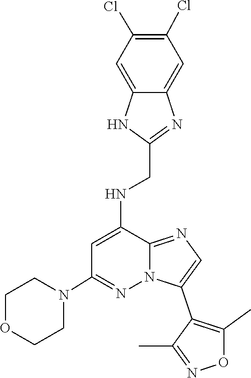 [SMILES: Cc1noc(C)c1-c1cnc2c(NCc3nc4cc(Cl)c(Cl)cc4[nH]3)cc(N3CCOCC3)nn12] | 100 | N-((5,6-dichloro-1H-benzo[d]imidazol-2- yl)methyl)-3-(3,5-dimethylisoxazol-4-yl)-6- morpholinoimidazo[1,2-b]pyridazin-8-amine | 513.35 |

The present disclosure further provides a method of synthesizing described herein, or a pharmaceutically acceptable salt, solvate or prodrug thereof.

With regard to the description of the synthetic methods described below and in the referenced synthetic methods that are used to prepare starting materials, it will be understood by those skilled in the art that all proposed reaction conditions, including choice of solvent, reaction atmosphere, reaction temperature, duration of the experiment and workup procedures, can be readily selected. Moreover, it will be understood by those skilled in the art that the functionality present on various portions of the molecule must be compatible with the reagents and reaction conditions utilized. An example of a particularly suitable method for synthesizing a compound of the present invention is shown as Scheme 1 below.

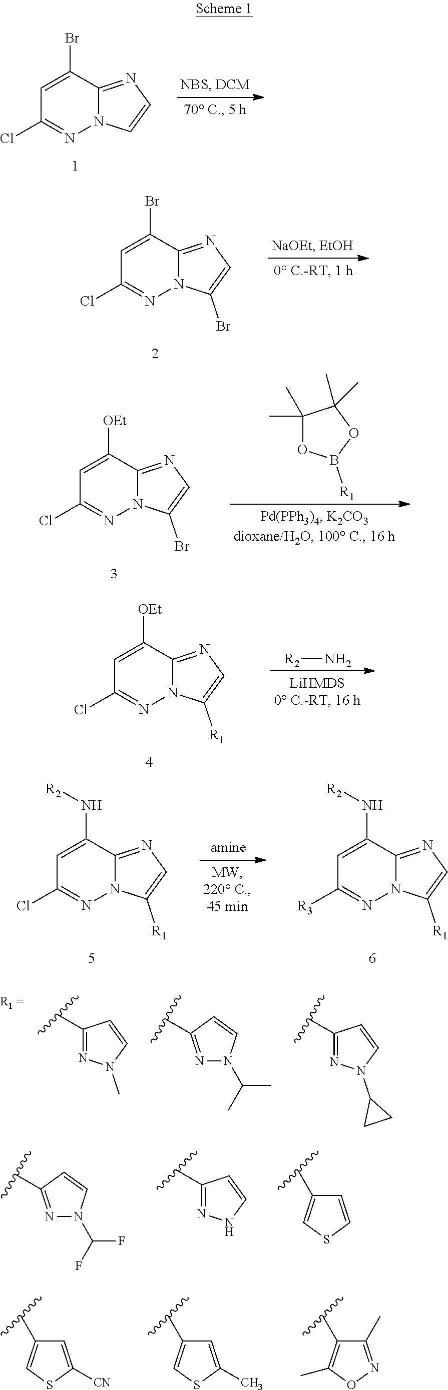

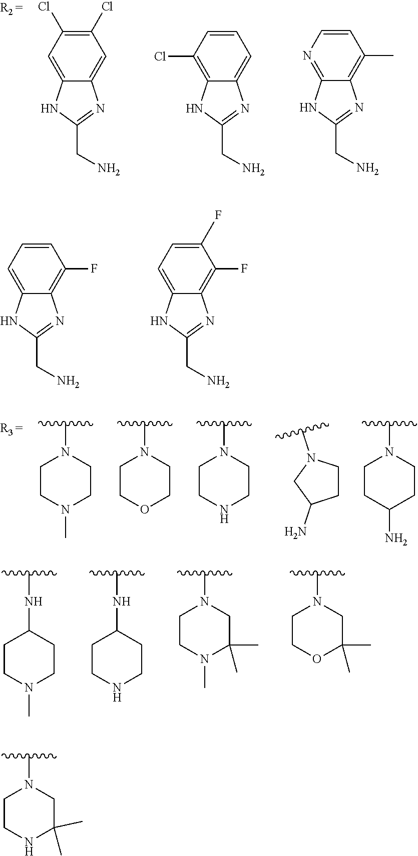

The present disclosure also includes compounds of Formula I with at least one desired isotopic substitution of an atom, at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P, ³²P, ³⁵S, ³⁶Cl, and ¹²⁵I, respectively. In one aspect, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed herein by substituting a readily available isotopically labeled reagent for a non-isotopically labeled reagent.

By way of general example and without limitation, isotopes of hydrogen, for example deuterium (²H) and tritium (³H) may optionally be used anywhere in described structures that achieves the desired result. Alternatively or in addition, isotopes of carbon, e.g., ¹³C and ¹⁴C, may be used. In one aspect, the isotopic substitution is replacing hydrogen with a deuterium at one or more locations on the molecule to improve the performance of the molecule as a drug, for example, the pharmacodynamics, pharmacokinetics, biodistribution, half-life, stability, AUC, Tₘₐₓ, Cₘₐₓ, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example deuterium substitutions, can be partial or complete. Partial deuterium substitution means that at least one hydrogen is substituted with deuterium. In certain aspects, the isotope is 80, 85, 90, 95, or 99% or more enriched in an isotope at any location of interest. In some aspects, deuterium is 80, 85, 90, 95, or 99% enriched at a desired location. Unless otherwise stated, the enrichment at any point is above natural abundance, and in an aspect is enough to alter a detectable property of the compounds as a drug in a human.

The compounds of the present disclosure may form a solvate with solvents (including water). Therefore, in one aspect, the invention includes a solvated form of the active compound. The term “solvate” refers to a molecular complex of a compound of the present invention (including a salt thereof) with one or more solvent molecules. Non-limiting examples of solvents are water, ethanol, dimethyl sulfoxide, acetone and other common organic solvents. The term “hydrate” refers to a molecular complex comprising a disclosed compound and water. Pharmaceutically acceptable solvates in accordance with the invention include those wherein the solvent of crystallization may be isotopically substituted, e.g., D₂O, de-acetone, or d₆-DMSO. A solvate can be in a liquid or solid form.

A “prodrug” as used herein means a compound which when administered to a host in vivo is converted into a parent drug. As used herein, the term “parent drug” means any of the presently described compounds herein. Prodrugs can be used to achieve any desired effect, including to enhance properties of the parent drug or to improve the pharmaceutic or pharmacokinetic properties of the parent, including to increase the half-life of the drug in vivo. Prodrug strategies provide choices in modulating the conditions for in vivo generation of the parent drug. Non-limiting examples of prodrug strategies include covalent attachment of removable groups, or removable portions of groups, for example, but not limited to, acylating, phosphorylation, phosphonylation, phosphoramidate derivatives, amidation, reduction, oxidation, esterification, alkylation, other carboxy derivatives, sulfoxy or sulfone derivatives, carbonylation, or anhydrides, among others. In certain aspects, the prodrug renders the parent compound more lipophilic. In certain aspects, a prodrug can be provided that has several prodrug moieties in a linear, branched, or cyclic manner. For example, non-limiting aspects include the use of a divalent linker moiety such as a dicarboxylic acid, amino acid, diamine, hydroxycarboxylic acid, hydroxyamine, di-hydroxy compound, or other compound that has at least two functional groups that can link the parent compound with another prodrug moiety, and is typically biodegradable in vivo. In some aspects, 2, 3, 4, or 5 prodrug biodegradable moieties are covalently bound in a sequence, branched, or cyclic fashion to the parent compound. Non-limiting examples of prodrugs according to the present disclosure are formed with: a carboxylic acid on the parent drug and a hydroxylated prodrug moiety to form an ester; a carboxylic acid on the parent drug and an amine prodrug to form an amide; an amino on the parent drug and a carboxylic acid prodrug moiety to form an amide; an amino on the parent drug and a sulfonic acid to form a sulfonamide; a sulfonic acid on the parent drug and an amino on the prodrug moiety to form a sulfonamide; a hydroxyl group on the parent drug and a carboxylic acid on the prodrug moiety to form an ester; a hydroxyl on the parent drug and a hydroxylated prodrug moiety to form an ester; a phosphonate on the parent drug and a hydroxylated prodrug moiety to form a phosphonate ester; a phosphoric acid on the parent drug and a hydroxylated prodrug moiety to form a phosphate ester; a hydroxyl on the parent drug and a phosphonate on the prodrug to form a phosphonate ester; a hydroxyl on the parent drug and a phosphoric acid prodrug moiety to form a phosphate ester; a carboxylic acid on the parent drug and a prodrug of the structure HO—(CH₂)₂—O—(C₂₋₂₄ alkyl) to form an ester; a carboxylic acid on the parent drug and a prodrug of the structure HO—(CH₂)₂—S—(C₂₋₂₄ alkyl) to form a thioester; a hydroxyl on the parent drug and a prodrug of the structure HO—(CH₂)₂—O—(C₂₋₂₄ alkyl) to form an ether; a hydroxyl on the parent drug and a prodrug of the structure HO—(CH₂)₂—O—(C₂₋₂₄ alkyl) to form an thioether; and a carboxylic acid, oxime, hydrazide, hydrazine, amine or hydroxyl on the parent compound and a prodrug moiety that is a biodegradable polymer or oligomer including but not limited to polylactic acid, polylactide-co-glycolide, polyglycolide, polyethylene glycol, polyanhydride, polyester, polyamide, or a peptide.

In some aspects, a prodrug is provided by attaching a natural or non-natural amino acid to an appropriate functional moiety on the parent compound, for example, oxygen, nitrogen, or sulfur, and typically oxygen or nitrogen, usually in a manner such that the amino acid is cleaved in vivo to provide the parent drug. The amino acid can be used alone or covalently linked (straight, branched or cyclic) to one or more other prodrug moieties to modify the parent drug to achieve the desired performance, such as increased half-life, lipophilicity, or other drug delivery or pharmacokinetic properties. The amino acid can be any compound with an amino group and a carboxylic acid, which includes an aliphatic amino acid, alkyl amino acid, aromatic amino acid, heteroaliphatic amino acid, heteroalkyl amino acid, heterocyclic amino acid, or heteroaryl amino acid.

### Methods

Further provided herein are methods of inhibiting a cyclin-dependent kinase, comprising contacting the cyclin dependent-kinase with an effective amount or concentration of a compound or pharmaceutical composition as disclosed herein. The cyclin-dependent kinase can be a cyclin-dependent kinase12 or 13 (CDK12 or CDK13). In some aspects of the methods disclosed herein, the CDK is disposed within the body tissue of a patient with cancer, inflammatory or myotonic dystrophy type 1 diseases, or a combination thereof.

Further provided herein are methods for the treatment of a disorder of uncontrolled cellular proliferation in a mammal comprising the step of administering to the mammal an effective amount of a compound as disclosed herein. In some examples, the disorder can be cancer. The methods can further comprise administering a second compound or composition. such as, for example, anti-cancer agents or anti-inflammatory agents. Additionally, the method can further comprise administering an effective amount of ionizing radiation to the subject.

Methods of killing a tumor cell are also provided herein. The methods comprise contacting a tumor cell with an effective amount of a compound or composition as disclosed herein. The methods can further include administering a second compound or composition (e.g., an anti-cancer agent or an anti-inflammatory agent) or administering an effective amount of ionizing radiation to the subject.

Also provided herein are methods of radiotherapy of tumors, comprising contacting the tumor with an effective amount of a compound or composition as disclosed herein and irradiating the tumor with an effective amount of ionizing radiation.

Also disclosed are methods for treating oncological disorders in a patient. In one aspect, an effective amount of one or more compounds or compositions disclosed herein is administered to a patient having an oncological disorder and who is in need of treatment thereof. The disclosed methods can optionally include identifying a patient who is or can be in need of treatment of an oncological disorder. The patient can be a human or other mammals, such as a primate (monkey, chimpanzee, ape, etc.), dog, cat, cow, pig, or horse, or other animals having an oncological disorder. Oncological disorders include, but are not limited to, cancer and/or tumors of the anus, bile duct, bladder, bone, bone marrow, bowel (including colon and rectum), breast, eye, gall bladder, kidney, mouth, larynx, esophagus, stomach, testis, cervix, head, neck, ovary, lung, mesothelioma, neuroendocrine, penis, skin, spinal cord, thyroid, vagina, vulva, uterus, liver, muscle, pancreas, prostate, blood cells (including lymphocytes and other immune system cells), and brain. Specific cancers contemplated for treatment include carcinomas, Kaposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma.

Other examples of cancers that can be treated according to the methods disclosed herein are adrenocortical carcinoma, adrenocortical carcinoma, cerebellar astrocytoma, basal cell carcinoma, bile duct cancer, bladder cancer, bone cancer, brain tumor, breast cancer, Burkitt's lymphoma, carcinoid tumor, central nervous system lymphoma, cervical cancer, chronic myeloproliferative disorders, colon cancer, cutaneous T-cell lymphoma, endometrial cancer, ependymoma, esophageal cancer, gallbladder cancer, gastric (stomach) cancer, gastrointestinal carcinoid tumor, germ cell tumor, glioma,, hairy cell leukemia, head and neck cancer, hepatocellular (liver) cancer, hypopharyngeal cancer, hypothalamic and visual pathway glioma, intraocular melanoma, retinoblastoma, islet cell carcinoma (endocrine pancreas), laryngeal cancer, lip and oral cavity cancer, liver cancer, medulloblastoma, Merkel cell carcinoma, squamous neck cancer with occult mycosis fungoides, myelodysplastic syndromes, myelogenous leukemia, nasal cavity and paranasal sinus cancer, nasopharyngeal cancer, neuroblastoma, non-small cell lung cancer, oral cancer, oropharyngeal cancer, osteosarcoma, ovarian cancer, pancreatic cancer, paranasal sinus and nasal cavity cancer, parathyroid cancer, penile cancer, pheochromocytoma, pineoblastoma and supratentorial primitive neuroectodermal tumor, pituitary tumor, plasma cell neoplasm/multiple myeloma, pleuropulmonary blastoma, prostate cancer, rectal cancer, renal cell (kidney) cancer, retinoblastoma, rhabdomyosarcoma, salivary gland cancer, Ewing's sarcoma, soft tissue sarcoma, Sezary syndrome, skin cancer, small cell lung cancer, small intestine cancer, supratentorial primitive neuroectodermal tumors, testicular cancer, thymic carcinoma, thymoma, thyroid cancer, transitional cell cancer of the renal pelvis and ureter, trophoblastic tumor, urethral cancer, uterine cancer, vaginal cancer, vulvar cancer, Waldenström's macroglobulinemia, and Wilms' tumor.

In some examples, the cancer is selected from breast cancer, brain cancer, cervical cancer, chronic myeloproliferative disorder, colorectal cancer, Ewing's sarcoma, gastrointestinal cancer, glioma, leukemia, lung cancer, lymphoma, endometrial cancer, melanoma, multiple myeloma, myelodysplastic syndrome, myeloproliferative neoplasm, pancreatic cancer, plasma cell neoplasm (myeloma), prostate cancer, ovarian cancer, osteosarcoma, skin cancer, testicular cancer, and thyroid cancer.

In some aspects, disclosed are methods for treating a tumor or tumor metastases in a subject by the administration to the subject a combination of at least one compound or composition as disclosed herein and at least one cancer immunotherapeutic agent. The disclosed compounds can be administered alone or in combination with a cancer immunotherapeutic agent. The subject can receive the therapeutic compositions before, during, or after surgical intervention to remove all or part of a tumor. Administration may be accomplished via direct immersion; systemic or localized intravenous (i.v.), intraperitoneal (i.p.), subcutaneous (s.c.), intramuscular (i.m.), or direct injection into a tumor mass; and/or by oral administration of the appropriate formulations.

In specific examples, the type of cancer is breast cancer or lung cancer.

Methods of treating a disease selected from inflammatory or myotonic dystrophy type diseases in a mammal comprising the step of administering to the mammal an effective amount of a compound as described herein are provided. In some aspects, the mammal is diagnosed with inflammatory or myotonic dystrophy type 1 disease.

### Administration

The disclosed compounds can be administered sequentially or simultaneously in separate or combined pharmaceutical formulations. When one or more of the disclosed compounds is combined with a second therapeutic agent, the dose of each compound can be either the same or differ from that when the compound is used alone. Appropriate doses will be readily appreciated by those skilled in the art.

The term “administration” and variants thereof (e.g., “administering” a compound) in reference to a compound of the invention means introducing the compound or a prodrug of the compound into the system of the animal in need of treatment. When a compound of the invention or prodrug thereof is combined with one or more other active agents (e.g., a cytotoxic agent, etc.), “administration” and its variants are each understood to include the concurrent and sequential introduction of the compound or prodrug thereof and other agents.

In vivo application of the disclosed compounds and compositions containing them can be accomplished by any suitable method and technique presently or prospectively known to those skilled in the art. For example, the disclosed compounds can be formulated in a physiologically- or pharmaceutically-acceptable form and administered by any suitable route known in the art, including oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration or at continuous or distinct intervals as readily determined by a person skilled in the art.

The compounds disclosed herein and compositions comprising them can also be administered utilizing liposome technology, slow-release capsules, implantable pumps, and biodegradable containers. These delivery methods can provide a uniform dosage over an extended period. The compounds can also be administered in their salt derivative forms or crystalline forms.

The compounds disclosed herein can be formulated according to known methods for preparing pharmaceutically acceptable compositions. Formulations are described in detail in many sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier to facilitate the effective administration of the compound. The compositions used can also be in a variety of forms. These include, for example, solid, semisolid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired treatment, compositions disclosed herein can advantageously comprise between about 0.1% and 99%, and especially, 1 and 15% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example, sealed ampoules and vials. The formulations can be stored in a freeze-dried (lyophilized) condition requiring only the condition of the sterile liquid carrier, for example, water for injections, before use. Extemporaneous injection solutions and suspensions can be prepared from sterile powder, granules, tablets, etc. It should be understood that in addition to the ingredients particularly mentioned above, the compositions disclosed herein can include other agents conventional in the art regarding the type of formulation in question.

Compounds disclosed herein and compositions comprising them can be delivered to a cell either through direct contact with the cell or via a carrier. Carriers for delivering compounds and compositions to cells are known in the art and include, for example, encapsulating the composition in a liposome moiety. Another means for delivering compounds and compositions disclosed herein to a cell comprises attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition, allowing the composition to be translocated across biological membranes. U.S. Application Publication No. 20020035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane: sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anti-cancer substances and/or with radiation and/or photodynamic therapy and/or with surgical treatment to remove a tumor. These other substances or treatments can be given simultaneously or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively.

Many tumors and cancers have a viral genome present in the tumor or cancer cells. For example, Epstein-Barr Virus (EBV) is associated with several mammalian malignancies. The compounds disclosed herein can also be used alone or in combination with anti-cancer or antiviral agents, such as ganciclovir, azidothymidine (AZT), lamivudine (3TC), etc., to treat patients infected with a virus that can cause cellular transformation and/or treat patients having a tumor or cancer that is associated with the presence of viral genome in the cells. The compounds disclosed herein can also be used in combination with viral-based treatments of oncologic disease. For example, the compounds can be used with mutant herpes simplex virus in the treatment of non-small cell lung cancer (Toyoizumi et al., “Combined therapy with chemotherapeutic agents and herpes simplex virus type IICP34.5 mutant (HSV-1716) in human non-small cell lung cancer,” Human Gene Therapy, 1999, 10 (18): 17).

Therapeutic application of compounds and/or compositions containing them can be accomplished by any suitable therapeutic method and technique presently or prospectively known to those skilled in the art. Further, compounds and compositions disclosed herein have use as starting materials or intermediates for the preparation of other useful compounds and compositions.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent or an assimilable edible carrier for oral delivery. They can be enclosed in hard or soft shell gelatin capsules, compressed into tablets, or incorporated directly with the food of the patient's diet. For oral therapeutic administration, the active compound can be combined with one or more excipients and used as ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch, or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to modify the solid unit dosage form's physical form. For instance, tablets, pills, or capsules can be coated with gelatin, wax, shellac, sugar, and the like. A syrup or elixir can contain the active compound, sucrose or fructose as a sweetening agent, methyl and propylparabens as preservatives, a dye, and flavorings such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts, hydrates, or analogs thereof, can be administered intravenously, intramuscularly, or intraperitoneally by infusion or injection. Solutions of the active agent or its salts can be prepared in water, optionally mixed with a non-toxic surfactant. Dispersions can also be prepared in glycerol, liquid polyethylene glycols, triacetin, oils, and mixtures thereof. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, non-toxic glyceryl esters, and suitable mixtures thereof. The proper fluidity can be maintained, for example, by the formation of liposomes, by the maintenance of the required particle size in the case of dispersions, or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by including agents that delay absorption, such as aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and freeze-drying techniques, which yield a powder of the active ingredient plus any additional desired ingredient present in the previously sterile-filtered solutions.

For topical administration, compounds and agents disclosed herein can be applied as a liquid or solid. However, it will generally be desirable to administer them topically to the skin as compositions, combined with a dermatologically acceptable carrier, which can be a solid or a liquid. Compounds, agents, and compositions disclosed herein can be applied topically to a subject's skin to reduce the size (and can include complete removal) of malignant or benign growths or treat an infection site. Compounds and agents disclosed herein can be applied directly to the growth or infection site. Preferably, the compounds and agents are applied to the growth or infection site in a formulation such as an ointment, cream, lotion, solution, tincture, or the like. Drug delivery systems for the delivery of pharmacological substances to dermal lesions can also be used, such as that described in U.S. Pat. No. 5,167,649.

Useful solid carriers include finely divided solids such as talc, clay, microcrystalline cellulose, silica, alumina, and the like. Useful liquid carriers include water, alcohols or glycols, or water-alcohol/glycol blends, where the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user. Examples of useful dermatological compositions which can be used to deliver a compound to the skin are disclosed in U.S. Pat. Nos. 4,608,392; 4,992,478; 4,559,157; and 4,820,508.

Useful dosages of the compounds and agents and pharmaceutical compositions disclosed herein can be determined by comparing their in vitro and in vivo activity in animal models. Methods for the extrapolation of effective dosages in mice, and other animals, to humans are known to the art; for example, see U.S. Pat. No. 4,938,949.

Also disclosed are pharmaceutical compositions that comprise a compound disclosed herein in combination with a pharmaceutically acceptable carrier. Pharmaceutical compositions adapted for oral, topical, or parenteral administration, comprising an amount of a compound, constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors, including the condition (health) of the subject, the body weight of the subject, kind of concurrent treatment, if any, frequency of treatment, therapeutic ratio, as well as the severity and stage of the pathological condition.

For the treatment of oncological disorders, compounds and agents and compositions disclosed herein can be administered to a patient in need of treatment before, after, or in combination with other antitumor or anti-cancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds and agents and compositions disclosed herein can be used in methods of treating cancer wherein the patient is to be treated or is or has been treated with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively. These other substances or radiation treatments can be given at the same or different times from the compounds disclosed herein. Examples of other suitable chemotherapeutic agents include, but are not limited to, altretamine, bleomycin, bortezomib (VELCADE), busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib (IRESSA), gemcitabine, hydroxyurea, idarubicin, ifosfamide, imatinib (GLEEVEC), irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uracil, temozolomide, thiotepa, tioguanine/thioguanine, topotecan, treosulfan, vinblastine, vincristine, vindesine, vinorelbine. In an exemplified aspect, the chemotherapeutic agent is melphalan. Examples of suitable immunotherapeutic agents include, but are not limited to, alemtuzumab, cetuximab (ERBITUX), gemtuzumab, iodine 131 tositumomab, rituximab, trastuzamab (HERCEPTIN). Cytotoxic agents include, for example, radioactive isotopes (e.g., I¹³¹, I¹²⁵, Y⁹⁰, P³², etc.), and toxins of bacterial, fungal, plant, or animal origin (e.g., ricin, botulinum toxin, anthrax toxin, aflatoxin, jellyfish venoms (e.g., box jellyfish), etc.) Also disclosed are methods for treating an oncological disorder comprising administering an effective amount of a compound and/or agent disclosed herein before, after, and/or in combination with administration of a chemotherapeutic agent, an immunotherapeutic agent, a radiotherapeutic agent, or radiotherapy.

### Kits

Kits for practicing the methods of the invention are further provided. By “kit” is intended any manufacture (e.g., a package or a container) comprising at least one reagent, e.g., any one of the compounds described in Table 1. The kit may be promoted, distributed, or sold as a unit for performing the methods of the present invention. Additionally, the kits may contain a package insert describing the kit and methods for its use. Any or all of the kit reagents may be provided within containers that protect them from the external environment, such as in sealed containers or pouches.

To provide for the administration of such dosages for the desired treatment, in some aspects, pharmaceutical compositions disclosed herein can comprise between about 0.1% and 45%, and especially, 1 and 15%, by weight of the total of one or more of the compounds based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight.

Also disclosed are kits that comprise a composition comprising a compound disclosed herein in one or more containers. The disclosed kits can optionally include pharmaceutically acceptable carriers and/or diluents. In one aspect, a kit includes one or more other components, adjuncts, or adjuvants as described herein. In another aspect, a kit includes one or more anti-cancer agents, such as those described herein. In one aspect, a kit includes instructions or packaging materials that describe how to administer a compound or composition of the kit. Containers of the kit can be of any suitable material, e.g., glass, plastic, metal, etc., and any suitable size, shape, or configuration. In one aspect, a compound and/or agent disclosed herein is provided in the kit as a solid, such as a tablet, pill, or powder form. In another aspect, a compound and/or agent disclosed herein is provided in the kit as a liquid or solution. In one aspect, the kit comprises an ampoule or syringe containing a compound and/or agent disclosed herein in liquid or solution form.

A number of aspects of the disclosure have been described. Nevertheless, it will be understood that various modifications may be made without departing from the spirit and scope of the invention. Accordingly, other aspects are within the scope of the following claims.

By way of non-limiting illustration, examples of certain aspects of the present disclosure are given below.

### EXAMPLES

The following examples are put forth so as to provide those of ordinary skill in the art with a complete disclosure and description of how the compounds, compositions, and/or methods claimed herein are made and evaluated and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric pressure.

### Example 1. Synthesis

General. All reagents were purchased from commercial suppliers and were used without further purification. Dichloromethane, ethanol, 1,4-dioxane, diethyl ether, N,N-dimethylformamide, acetic acid and ethyl acetate were dried by being passed through a column of desiccant (activated A-1 alumina). N-bromo succinimide, sodium ethoxide (21% w/w in ethanol) and LiHMDS (1M in THF) was purchased from Sigma-Aldrich and used as such. Reactions were either monitored by thin layer chromatography or analytical LC-MS. Thin layer chromatography was performed on Kieselgel 60 F254 glass plates pre-coated with a 0.25 mm thickness of silica gel. TLC plates were visualized with UV light and/or by staining with ninhydrin solution. Normal phase column chromatography was performed on a Biotage Selekt automated flash system. Compounds were loaded onto pre-filled cartridges filled with KP-Sil 50 μm irregular silica. For microwave reactions, a Biotage Initiator Microwave system was used. Final products were isolated by reverse-phase HPLC using Waters HPLC system with UV detector, with Atlantis T3 OBD Prep Column, 100 Å, 5 μm, 19 mm×150 mm. Compounds were eluted using a gradient elution of 90/10 to 0/100 A/B over 20 min at a flow rate of 20.0 mL/min, where solvent A was water (+0.1% formic acid), and solvent B was acetonitrile.

The structures of all compounds were verified via ¹H NMR, ¹⁹F NMR and LCMS. The purity of isolated products was determined using an LC-MS instrument (Agilent 1290 Infinity series LC with single quadrupole MSD system, AP-ESI Ion Source) equipped with Kinetex® 1.7 μm C18 100 Å, LC Column 50×2.1 mm, Ea (Phenomenex) column. Elution was performed using the following conditions: 2% (v/v) acetonitrile (+0.1% FA) in 98% (v/v) H2O (+0.1% FA), ramped to 98% acetonitrile over 4.0 min, and holding at 98% acetonitrile for 0.5 min with a flow rate of 0.6 mL/min; UV absorption was detected from 200 to 950 nm using a diode array detector. The purity of each compound was ≥95% based on this analysis.

NMR spectra were recorded at ambient temperature on a 500 MHz Bruker NMR spectrometer in DMSO-d₆. All ¹H NMR data are reported in parts per million (ppm) downfield of TMS and were measured relative to the signals for dimethyl sulfoxide (2.50 ppm). ¹⁹F NMR experiments were performed with ¹H decoupling. Data for H NMR are reported as follows: chemical shift (δ, ppm), multiplicity (s=singlet, d=doublet, t=triplet, q=quartet, m=multiplet), integration, and coupling constant (Hz). NMR data was analyzed and processed by using MestReNova software.

### 3,8-dibromo-6-chloroimidazo[1,2-b]pyridazine (2)

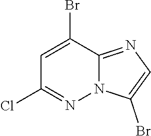 [SMILES: Clc1cc(Br)c2ncc(Br)n2n1]

A solution of 8-bromo-6-chloroimidazo[1,2-b]pyridazine (10.0 g, 43.02 mmol) and NBS (8.42 g, 47.32 mmol) in dichloromethane (80 mL) was heated to 70° C. in a sealed tube. After completion, solvent was evaporated and water was added to the residue resulting in an off-white precipitate which was filtered and dried to afford 3,8-dibromo-6-chloroimidazo[1,2-b]pyridazine (12.0 g, 89.59%) as an off-white solid. ¹H NMR (500 MHz, DMSO) δ 8.21 (d, J=10.5 Hz, 1H), 7.82 (d, J=6.1 Hz, 1H). LCMS (m/z): 311.8 (M+1), Rt: 2.577 min.

### 3-bromo-6-chloro-8-ethoxyimidazo[1,2-b]pyridazine (3)

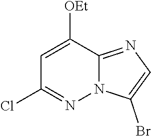 [SMILES: CCOc1cc(Cl)nn2c(Br)cnc12]

To a solution of 3,8-dibromo-6-chloroimidazo[1,2-b]pyridazine (12.0 g, 38.54 mmol) in ethanol (50 mL) at 0° C. was added sodium ethanolate (28.0 mL, 77.08 mmol) dropwise and stirred at room temperature for 1 h. After completion, solvent was evaporated. The residue was treated with IN NH₄Cl in water and extracted with CH₂Cl₂ (3×50 mL). The combined organic layers were concentrated, and the residue was purified by column chromatography on silica gel (20%-30% ethyl acetate in hexanes) to afford 3-bromo-6-chloro-8-ethoxyimidazo|1,2-b| pyridazine (8.0 g, 75.07%) as a pale-orange solid. ¹H NMR (500 MHz, DMSO) δ 7.79 (s, 1H), 7.00 (s, 1H), 4.40 (q, J=7.0 Hz, 2H), 1.44 (t, J=7.0 Hz, 3H). LCMS (m/z): 277.9 (M+2), Rt: 2.531 min.

General Procedure for Step 3 (3a-3i).

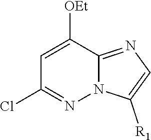

Argon gas was bubbled to a mixture of 3-bromo-6-chloro-8-ethoxyimidazo[1,2-b]pyridazine (1.0 mmol), boronic ester (1.10 mmol), K₂CO₃ (2.0 mmol), 1,4-dioxane (8v) and water (2v) for 10 min. To this mixture, Pd(PPh₃)₄ (0.1 mmol) was added, and the reaction was heated to 90° C. for 16 h. After completion, the reaction was cooled to room temperature and was transferred to separatory funnel containing brine (50 mL) and the aqueous layer was extracted with ethyl acetate (3×50 mL). The combined organic layers were dried over sodium sulphate and concentrated under reduced pressure. The residue was purified by column chromatography on silica gel (20%-50% ethyl acetate in hexanes).

General Procedure for Step 4 (4a-4c).

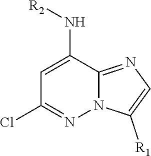

To a solution of step-3 compound (1.0 mmol) and benzimidazole (1.0 mmol) at 0° C. under argon atm was added LiHMDS (4.0 mmol, 1M in THF) dropwise. The reaction turned to dark brown. The solution was allowed to warm to room temperature and stirred for 16 h. After completion, the reaction was quenched with brine (30 mL) and the aqueous layer was extracted with ethyl acetate (3×30 mL). The combined organic layers were dried over sodium sulphate and concentrated under reduced pressure. The residue was purified by column chromatography on silica gel (2%-5% methanol in dichloromethane).

### General Procedure for Final Reactions, Step 5.

1.5 To a flame-dry microwave vial, 50 mg of SM (4a-4e, 1 mmol) was added 1-methyl piperazine (2 mL) for methyl piperazine analogues and morpholine (2 mL) for morpholine analogues were heated to 220° C. in microwave for 45 min. After completion, solvent was removed under reduced pressure and purified by PREP HPLC purification, where solvent A was water (+0.1% formic acid), and solvent B was acetonitrile. Pure fraction was concentrated under reduced pressure and the residue was triturated with diethyl ether and 20 dried in high vacuo.

### 6-chloro-8-ethoxy-3-(1-methyl-1H-pyrazol-4-yl) imidazo[1,2-b]pyridazine (3a)

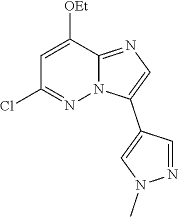 [SMILES: CCOc1cc(Cl)nn2c(-c3cnn(C)c3)cnc12]

Following the general procedure step 3, 3-bromo-6-chloro-8-ethoxyimidazo[1,2-b]pyridazine (2.0 g, 7.23 mmol), 1-methyl-4-(4,4,5,5-tetramethyl-1,3,2-dioxaborolan-2-yl)-1H-pyrazole (1.66 g, 7.96 mmol), K₂CO₃ (1.50 g, 10.85 mmol), 1,4-dioxane (16 mL) and water (4 mL) for 10 min. To this mixture, Pd(PPh₃)₄ (836 mg) was added, and the reaction was heated to 90° C. for 16 h. After completion, the reaction was cooled to room temperature and was transferred to separatory funnel containing brine (50 mL) and the aqueous layer was extracted with ethyl acetate (3×50 mL). The combined organic layers were dried over sodium sulphate and concentrated under reduced pressure. The residue was purified by column chromatography on silica gel (20%-50% ethyl acetate in hexanes) to afford 3, 3-bromo-6-chloro-8-ethoxyimidazo[1,2-b]pyridazine (1.50 g, 74.68%) as an off-white solid. ¹H NMR (500 MHz, DMSO) δ 8.35 (s, 1H), 8.07 (s, 1H), 7.95 (s, 1H), 6.92 (s, 1H), 4.42 (q, J=7.0 Hz, 2H), 3.94 (s, 3H), 1.45 (t, J=7.0 Hz, 3H). LCMS (m/z): 278.0 (M+1), Rt: 2.226 min.

### 6-chloro-N-((4,5-difluoro-1H-benzo[d]imidazol-2-yl)methyl)-3-(1-methyl-1H-pyrazol-4-yl) imidazo[1,2-b]pyridazin-8-amine (4)

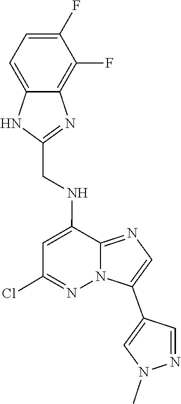 [SMILES: Cn1cc(-c2cnc3c(NCc4nc5c(F)c(F)ccc5[nH]4)cc(Cl)nn23)cn1]

Following the general procedure, 3, 3-bromo-6-chloro-8-ethoxyimidazo[1,2-b]pyridazine (0.5 g. 1.80 mmol) and (4,5-difluoro-1H-benzo[d]imidazol-2-yl) methanamine (330 mg, 1.80 mmol) at 0° C. under argon atm was added LiHMDS (7.20 mL, 7.20 mmol, 1M in THF) dropwise. The reaction turned to dark brown. The solution was allowed to warm to room temperature and stirred for 16 h. After completion, the reaction was quenched with brine (30 mL) and the aqueous layer was extracted with ethyl acetate (3×30 mL). The combined organic layers were dried over sodium sulphate and concentrated under reduced pressure. The residue was purified by column chromatography on silica gel (2%-5% methanol in dichloromethane) to afford 6-chloro-N-((4,5-difluoro-1H-benzo[d]imidazol-2-yl)methyl)-3-(1-methyl-1H-pyrazol-4-yl) imidazo[1,2-b]pyridazin-8-amine (0.5 g, 66.95%) as an off-white solid. LCMS (m/z): 415.0 (M+1), Rt: 2.540 min.

### N-((4,5-difluoro-1H-benzo[d]imidazol-2-yl)methyl)-3-(1-methyl-1H-pyrazol-4-yl)-6-(4-methylpiperazin-1-yl) imidazo[1,2-b]pyridazin-8-amine (5)

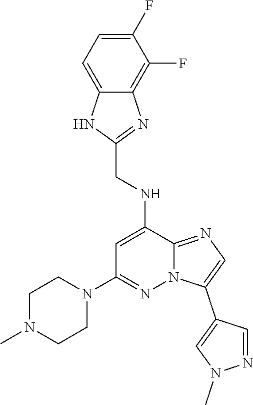 [SMILES: CN1CCN(c2cc(NCc3nc4c(F)c(F)ccc4[nH]3)c3ncc(-c4cnn(C)c4)n3n2)CC1]

Following the general procedure, 6-chloro-N-((4,5-difluoro-1H-benzo[d]imidazol-2-yl)methyl)-3-(1-methyl-1H-pyrazol-4-yl) imidazo[1,2-b]pyridazin-8-amine (50 mg, 0.120 mmol), 1-methyl piperazine (2 mL) were heated to 220° C. in microwave for 45 min. After completion, solvent was removed under reduced pressure and purified by PREP HPLC purification, where solvent A was water (+0.1% formic acid), and solvent B was acetonitrile. Pure fraction was concentrated under reduced pressure and the residue was triturated with diethyl ether and dried in high vacuo to afford N-((4,5-difluoro-1H-benzo[d]imidazol-2-yl)methyl)-3-(1-methyl-1H-pyrazol-4-yl)-6-(4-methylpiperazin-1-yl) imidazo[1,2-b]pyridazin-8-amine (28.0 mg, 48.54%) as a pale-yellow solid. ¹H NMR (500 MHz, DMSO) δ 8.25 (s, 1H), 8.03 (s, 1H), 7.62 (s, 1H), 7.52 (t, J=6.2 Hz, 1H), 7.26 (dd, J=9.0, 3.7 Hz, 1H), 7.21-7.14 (m, 1H), 6.02 (s, 1H), 4.81 (d, J=6.1 Hz, 2H), 3.91 (s, 3H), 3.39 (t, J=5.0 Hz, 4H), 2.41 (t, J=5.0 Hz, 4H), 2.20 (s, 3H). ¹⁹F NMR (471 MHz, DMSO) δ −150.07, −155.50 (d, J=21.2 Hz). LCMS (m/z): 479.1 (M+1), Rt: 1.769 min.

### N-((4,5-difluoro-1H-benzo[d]imidazol-2-yl)methyl)-3-(1-methyl-1H-pyrazol-4-yl)-6-morpholinoimidazo[1,2-b]pyridazin-8-amine (6)

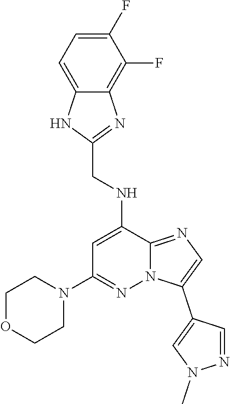 [SMILES: Cn1cc(-c2cnc3c(NCc4nc5c(F)c(F)ccc5[nH]4)cc(N4CCOCC4)nn23)cn1]

Synthesized according to procedure described for 5. Yield (5.0 mg, 8.91%), Off-white solid. ¹H NMR (500 MHz, DMSO) δ 12.71 (s, 1H), 8.27 (s, 1H), 8.04 (s, 1H), 7.64 (s, 1H), 7.57 (s, 1H), 7.27-7.15 (m, 2H), 6.04 (s, 1H). 4.82 (d, J=6.2 Hz, 2H), 3.92 (s, 3H), 3.72 (t. J=4.8 Hz, 4H), 3.37 (q, J=5.8, 4.7 Hz, 4H). ¹⁹F NMR (471 MHz, DMSO) δ −150.12 (d, J=21.4 Hz), −155.50 (d, J=21.1 Hz). LCMS (m/z): 466.1 (M+1), Rt: 2.176 min.

### 6-chloro-N-((5,6-dichloro-1H-benzo[d]imidazol-2-yl)methyl)-3-(1-methyl-1H-pyrazol-4-yl) imidazo[1,2-b]pyridazin-8-amine (7)

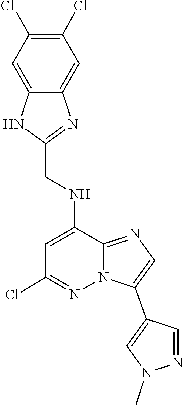 [SMILES: Cn1cc(-c2cnc3c(NCc4nc5cc(Cl)c(Cl)cc5[nH]4)cc(Cl)nn23)cn1]

Synthesized according to procedure described for 4. Yield (150 mg, 46.52%). Pale brown solid. LCMS (m/z): 448.9 (M+1), Rt: 2.793 min.

### N-((5,6-dichloro-1H-benzo[d]imidazol-2-yl)methyl)-3-(1-methyl-1H-pyrazol-4-yl)-6-(4-methylpiperazin-1-yl) imidazo[1,2-b]pyridazin-8-amine (8)

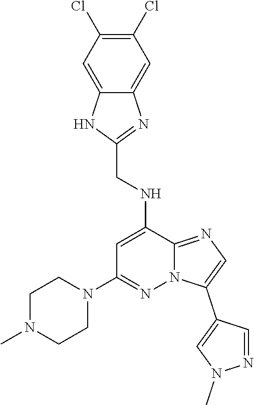 [SMILES: CN1CCN(c2cc(NCc3nc4cc(Cl)c(Cl)cc4[nH]3)c3ncc(-c4cnn(C)c4)n3n2)CC1]

Synthesized according to procedure described for 5. Yield (23 mg, 40.27%), Off-white solid. ¹H NMR (500 MHz, DMSO) δ 8.25 (s, 1H), 8.03 (s, 1H), 7.76 (s, 2H), 7.61 (s, 1H), 7.51 (t, J=6.1 Hz, 1H), 5.99 (s, 1H), 4.80 (d, J=6.1 Hz, 2H), 3.91 (s, 3H), 3.38 (t, J=3.5 Hz, 4H), 2.40 (t, J=5.0 Hz, 4H), 2.20 (s, 3H). LCMS (m/z): 512.1 (M+1), Rt: 1.969 min.

### N-((5,6-dichloro-1H-benzo[d]imidazol-2-yl)methyl)-3-(1-methyl-1H-pyrazol-4-yl)-6-morpholinoimidazo[1,2-b]pyridazin-8-amine (9)

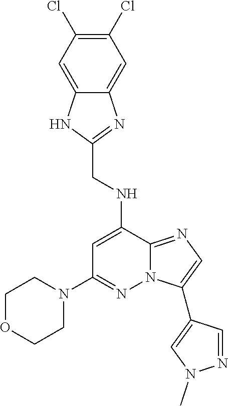 [SMILES: Cn1cc(-c2cnc3c(NCc4nc5cc(Cl)c(Cl)cc5[nH]4)cc(N4CCOCC4)nn23)cn1]

Synthesized according to procedure described for 5. Yield (21 mg, 37.73%), Off-white solid. ¹H NMR (500 MHz, DMSO) § 12.54 (s, 1H), 8.26 (s, 1H), 8.03 (s, 1H), 7.76 (s, 2H), 7.63 (s, 1H), 7.56 (t, J=6.2 Hz, 1H), 6.01 (s, 1H), 4.81 (d, J=6.2 Hz, 2H), 3.91 (s, 3H), 3.76-3.68 (m, 4H), 3.36 (t, J=4.8 Hz, 4H). LCMS (m/z): 498.1 (M+1), Rt: 2.415 min.

### 6-chloro-3-(1-methyl-1/-pyrazol-4-yl)-N-((7-methyl-3H-imidazo[4,5-b]pyridin-2-yl)methyl) imidazo[1,2-b]pyridazin-8-amine (10)

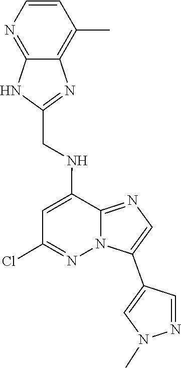 [SMILES: Cc1ccnc2[nH]c(CNc3cc(Cl)nn4c(-c5cnn(C)c5)cnc34)nc12]

Synthesized according to procedure described for 4. Yield (140 mg, 48.68%). Off-white solid. LCMS (m/z): 394.0 (M+1), Rt: 1.902 min.

### 3-(1-methyl-1H-pyrazol-4-yl)-N-((7-methyl-3H-imidazo[4,5-b]pyridin-2-yl)methyl)-6-(4-methylpiperazin-1-yl) imidazo[1,2-b]pyridazin-8-amine (11)

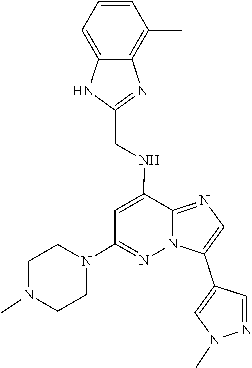 [SMILES: Cc1cccc2[nH]c(CNc3cc(N4CCN(C)CC4)nn4c(-c5cnn(C)c5)cnc34)nc12]

Synthesized according to procedure described for 5. Yield (23 mg, 56.56%), Off-white solid. ¹H NMR (500 MHz, DMSO) δ 8.25 (s, 1H), 8.15 (s, 1H), 8.02 (s, 1H), 7.61 (s, 1H), 7.43 (t, J=6.0 Hz, 1H), 7.02 (d, J=4.9 Hz, 1H), 6.06 (s, 1H), 4.79 (d, J=5.9 Hz, 2H), 3.91 (s, 3H), 3.42-3.37 (m, 5H), 2.52 (s, 3H), 2.43 (t, J=5.0 Hz, 4H), 2.22 (s, 3H). LCMS (m/z): 458.2 (M+1), Rt: 1.432 min.

### 3-(1-methyl-1H-pyrazol-4-yl)-N-((7-methyl-3H-imidazo[4,5-b]pyridin-2-yl)methyl)-6-morpholinoimidazo[1,2-b]pyridazin-8-amine (12)

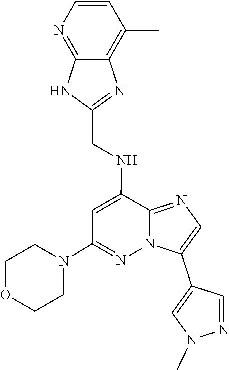 [SMILES: Cc1ccnc2[nH]c(CNc3cc(N4CCOCC4)nn4c(-c5cnn(C)c5)cnc34)nc12]

Synthesized according to procedure described for 5. Yield (11 mg, 27.85%), pale-brown solid. ¹H NMR (500 MHz, DMSO) δ 12.95 (s, 1H), 8.26 (s, 1H), 8.15 (d, J=36.8 Hz, 1H), 8.03 (s, 1H), 7.63 (s, 1H), 7.47 (t, J=6.1 Hz, 1H), 7.02 (d, J=4.9 Hz, 1H), 6.08 (s, 1H), 4.79 (s, 2H), 3.91 (s, 3H), 3.73 (t, J=4.8 Hz, 4H), 3.37 (t, J=4.8 Hz, 4H). LCMS (m/z): 445.2 (M+1), Rt: 1.725 min.

### 6-chloro-8-ethoxy-3-(1-isopropyl-1H-pyrazol-4-yl) imidazo[1,2-b]pyridazine (3b)

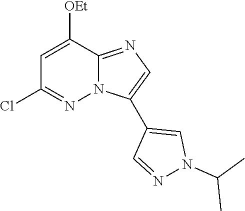 [SMILES: CCOc1cc(Cl)nn2c(-c3cnn(C(C)C)c3)cnc12]

Following the general procedure step 3, 3-bromo-6-chloro-8-ethoxyimidazo[1,2-b]pyridazine (1.50 g, 5.42 mmol), 1-isopropyl-4-(4,4,5,5-tetramethyl-1,3,2-dioxaborolan-2-yl)-1H-pyrazole (1.42 g, 5.97 mmol), K₂CO₃ (1.87 g, 13.56 mmol), 1,4-dioxane (16 mL) and water (4 mL) for 10 min. To this mixture, Pd(PPh₃)₄ (333 mg) was added, and the reaction was heated to 90° C. for 16 h. After completion, the reaction was cooled to room temperature and was transferred to separatory funnel containing brine (50 mL) and the aqueous layer was extracted with ethyl acetate (3×50 mL). The combined organic layers were dried over sodium sulphate and concentrated under reduced pressure. The residue was purified by column chromatography on silica gel (20%-50% ethyl acetate in hexanes) to afford 6-chloro-8-ethoxy-3-(1-isopropyl-1H-pyrazol-4-yl) imidazo[1,2-b]pyridazine (1.20 g, 72.35%) as a pale-yellow gummy solid. ¹H NMR (500 MHz, DMSO) δ 8.39 (s, 1H), 8.10 (s, 1H), 7.94 (s, 1H), 6.91 (s, 1H), 4.63 (p, J=6.7 Hz, 1H), 4.41 (q, J=6.9 Hz, 2H), 3.92 (s, 2H), 1.47 (d, J=6.5 Hz, 6H), 1.07 (s, 9H). LCMS (m/z): 306.1 (M+1), Rt: 2.611 min.

### 6-chloro-N-((5,6-dichloro-1H-benzo[d]imidazol-2-yl)methyl)-3-(1-isopropyl-1H-pyrazol-4-yl) imidazo[1,2-b]pyridazin-8-amine (13)

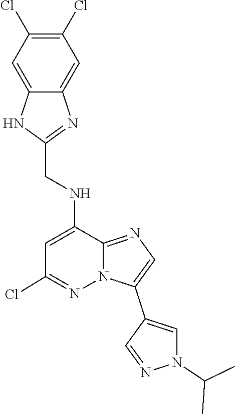 [SMILES: CC(C)n1cc(-c2cnc3c(NCc4nc5cc(Cl)c(Cl)cc5[nH]4)cc(Cl)nn23)cn1]

Synthesized according to procedure described for 4. Yield (220 mg, 47.13%). Pale-orange solid. ¹H NMR (500 MHz, DMSO) δ 8.36 (d, J=5.8 Hz, 1H), 8.08 (d, J=4.1 Hz, 1H), 7.85 (s, 1H), 7.67-7.58 (m, 1H), 6.31 (s, 1H), 4.86 (s, 1H), 4.62 (dd, J=13.3, 6.6 Hz, 1H), 1.52-1.41 (m, 6H). LCMS (m/z): 477.0 (M+1), Rt: 3.086 min.

### N-((5,6-dichloro-1H-benzo[d]imidazol-2-yl)methyl)-3-(1-isopropyl-1H-pyrazol-4-yl)-6-(4-methylpiperazin-1-yl) imidazo[1,2-b]pyridazin-8-amine (14)

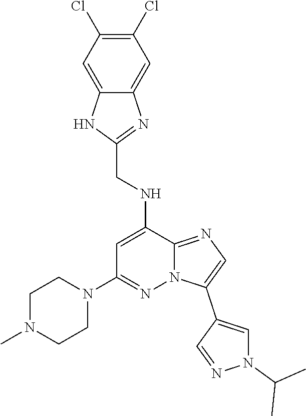 [SMILES: CC(C)n1cc(-c2cnc3c(NCc4nc5cc(Cl)c(Cl)cc5[nH]4)cc(N4CCN(C)CC4)nn23)cn1]

Synthesized according to procedure described for 5. Yield (10 mg, 17.64%), pale-brown solid. ¹H NMR (500 MHz, DMSO) δ 8.33 (s, 1H), 8.07 (s, 1H), 7.77 (s, 1H), 7.61 (s, 1H), 7.51 (t, J=6.2 Hz, 1H), 5.99 (s, 1H), 4.81 (d, J=6.1 Hz, 2H), 4.57 (p, J=6.7 Hz, 1H), 3.40-3.37 (m, 4H), 2.41 (t, J=5.0 Hz, 4H), 2.20 (s, 3H), 1.46 (d, J=6.7 Hz, 6H). LCMS (m/z): 537.1 (M-2), Rt: 2.104 min.

### N-((5,6-dichloro-1H-benzo[d]imidazol-2-yl)methyl)-3-(1-isopropyl-1H-pyrazol-4-yl)-6-morpholinoimidazo[1,2-b]pyridazin-8-amine (15)

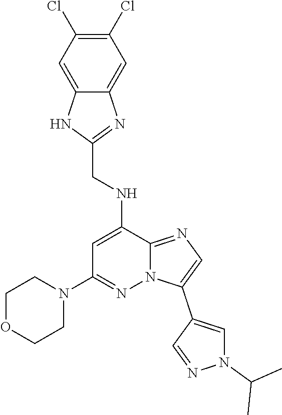 [SMILES: CC(C)n1cc(-c2cnc3c(NCc4nc5cc(Cl)c(Cl)cc5[nH]4)cc(N4CCOCC4)nn23)cn1]

Synthesized according to procedure described for 5. Yield (8 mg, 10.33%), pale-brown solid. ¹H NMR (500 MHz, DMSO) δ 12.61 (s, 1H), 8.33 (s, 1H), 8.08 (s, 1H), 7.84 (d, J=8.9 Hz, 1H), 7.70 (s, 1H), 7.63 (s, 1H), 7.55 (t, J=6.2 Hz, 1H), 6.01 (s, 1H), 4.81 (d, J=6.2 Hz, 2H), 4.57 (p, J=6.7 Hz, 1H), 3.79-3.63 (m, 4H), 3.36 (dd, J=5.8, 3.8 Hz, 4H), 1.46 (d, J=6.7 Hz, 6H). LCMS (m/z): 527.1 (M+1), Rt: 2.650 min.

### 6-chloro-N-((7-chloro-1H-benzo[d]imidazol-2-yl)methyl)-3-(1-isopropyl-1H-pyrazol-4-yl) imidazo[1,2-b]pyridazin-8-amine (16)

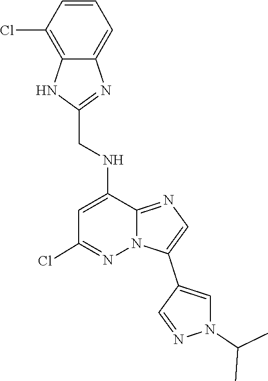 [SMILES: CC(C)n1cc(-c2cnc3c(NCc4nc5cccc(Cl)c5[nH]4)cc(Cl)nn23)cn1]

Synthesized according to procedure described for 4. Yield (180 mg, 49.88%). Pale-orange solid. ¹H NMR (500 MHz, DMSO) δ 8.36 (s, 1H), 8.07 (s, 1H), 7.85 (d, J=4.5 Hz, 1H), 7.66-7.58 (m, 1H), 7.55 (td, J=7.4, 3.0 Hz, 1H), 7.42 (d, J=7.9 Hz, 1H), 7.16 (t, J=7.9 Hz, 1H), 4.88 (s, 1H), 4.62 (p, J=6.6 Hz, 2H), 1.46 (dd, J=6.7, 2.4 Hz, 6H). LCMS (m/z): 442.1 (M+1), Rt: 2.796 min.

### N-((7-chloro-1H-benzo[d]imidazol-2-yl)methyl)-3-(1-isopropyl-1H-pyrazol-4-yl)-6-(4-methylpiperazin-1-yl) imidazo[1,2-b]pyridazin-8-amine (17)

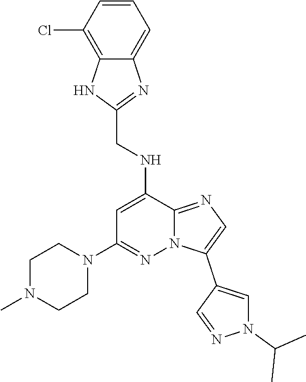 [SMILES: CC(C)n1cc(-c2cnc3c(NCc4nc5cccc(Cl)c5[nH]4)cc(N4CCN(C)CC4)nn23)cn1]

Synthesized according to procedure described for 5. Yield (6 mg, 7.49%), pale-yellow solid. ¹H NMR (500 MHz, DMSO) δ 12.69 (s, 1H), 8.33 (s, 1H), 8.06 (s, 1H), 7.61 (s, 1H), 7.53 (s, 1H), 7.43 (s, 1H), 7.23 (d, J=7.7 Hz, 1H), 7.15 (t, J=7.8 Hz, 1H), 6.09 (s, 1H), 4.81 (d, J=6.0 Hz, 2H), 4.56 (p, J=6.7 Hz, 1H), 3.39 (dd, J=6.3, 3.8 Hz, 4H), 2.41 (t, J=5.0 Hz, 4H), 2.20 (s, 3H), 1.45 (d, J=6.7 Hz, 6H). LCMS (m/z): 506.2 (M+1), Rt: 1.895 min.

### N-((7-chloro-1H-benzo[d]imidazol-2-yl)methyl)-3-(1-isopropyl-1H-pyrazol-4-yl)-6-morpholinoimidazo[1,2-b]pyridazin-8-amine (18)

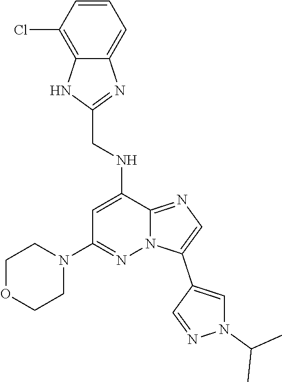 [SMILES: CC(C)n1cc(-c2cnc3c(NCc4nc5cccc(Cl)c5[nH]4)cc(N4CCOCC4)nn23)cn1]

Synthesized according to procedure described for 5. Yield (3 mg, 3.84%), pale-yellow solid. ¹H NMR (500 MHz, DMSO) δ 12.69 (s, 1H), 8.33 (s, 1H), 8.07 (s, 1H), 7.60 (d, J=23.1 Hz, 2H), 7.42 (s, 1H), 7.23 (d, J=7.7 Hz, 1H), 7.15 (t, J=7.9 Hz, 1H), 6.10 (s, 1H), 4.82 (d, J=6.1 Hz, 2H), 4.58 (h, J=6.8 Hz, 1H), 3.72 (t, J=4.6 Hz, 4H), 3.37 (t, J=4.8 Hz, 4H), 1.45 (d, J=6.5 Hz, 6H). LCMS (m/z): 492.1 (M+1), Rt: 2.405 min.

### 6-chloro-3-(1-isopropyl-1H-pyrazol-4-yl)-N-((7-methyl-3H-imidazo[4,5-b]pyridin-2-yl)methyl) imidazo[1,2-b]pyridazin-8-amine (19)

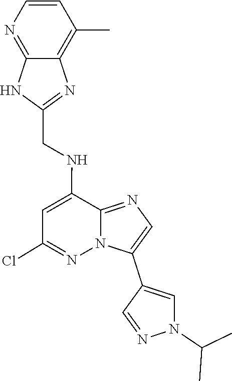 [SMILES: Cc1ccnc2[nH]c(CNc3cc(Cl)nn4c(-c5cnn(C(C)C)c5)cnc34)nc12]

Synthesized according to procedure described for 4. Yield (220 mg, 53.15%). Pale-orange solid. ¹H NMR (500 MHz, DMSO) δ 8.35 (s, 1H), 8.31 (q, J=8.0, 7.2 Hz, 1H), 8.07 (s, 1H), 7.85 (s, 1H), 7.03 (d, J=4.9 Hz, 1H), 6.33 (s, 1H), 4.86 (s, 2H), 4.64-4.60 (m, 1H), 1.46 (d, J=6.7 Hz, 6H), 1.07 (s, 3H). LCMS (m/z): 422.1 (M+1), Rt: 2.184 min.

### 3-(1-isopropyl-1H-pyrazol-4-yl)-N-((7-methyl-3H-imidazo[4,5-b]pyridin-2-yl)methyl)-6-(4-methylpiperazin-1-yl) imidazo[1,2-b]pyridazin-8-amine (20)

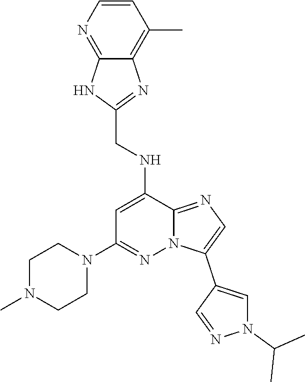 [SMILES: Cc1ccnc2[nH]c(CNc3cc(N4CCN(C)CC4)nn4c(-c5cnn(C(C)C)c5)cnc34)nc12]

Synthesized according to procedure described for 5. Yield (11 mg, 19.11%), pale-brown solid. ¹H NMR (500 MHz, DMSO) δ 8.33 (s, 1H), 8.14 (s, 1H), 8.06 (s, 1H), 7.61 (s, 1H), 7.42 (t, J=6.0 Hz, 1H), 7.02 (d, J=4.9 Hz, 1H), 6.07 (s, 1H), 4.79 (d, J=5.9 Hz, 2H), 4.56 (p, J=6.6 Hz, 1H), 3.40 (t, J=4.7 Hz, 5H), 2.52 (s, 3H), 2.42 (t, J=5.0 Hz, 4H), 2.21 (s, 3H), 1.45 (d, J=6.7 Hz, 6H). LCMS (m/z): 486.2 (M+1), Rt: 1.595 min.

### 3-(1-isopropyl-1H-pyrazol-4-yl)-N-((7-methyl-3H-imidazo[4,5-b]pyridin-2-yl)methyl)-6-morpholinoimidazo[1,2-b]pyridazin-8-amine (21)

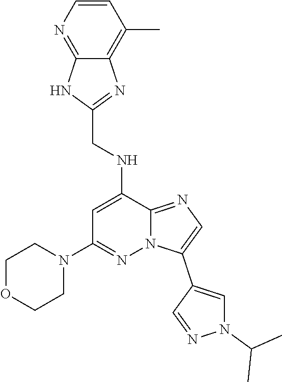 [SMILES: Cc1ccnc2[nH]c(CNc3cc(N4CCOCC4)nn4c(-c5cnn(C(C)C)c5)cnc34)nc12]

Synthesized according to procedure described for 5. Yield (6 mg, 10.71%), pale-brown solid. ¹H NMR (500 MHz, DMSO) δ 12.91 (s, 1H), 8.33 (s, 1H), 8.14 (s, 1H), 8.07 (s, 1H), 7.62 (s, 1H), 7.47 (t, J=6.0 Hz, 1H), 7.02 (d. J=4.9 Hz, 1H), 6.08 (s, 1H), 4.80 (d, J=6.0 Hz, 2H), 4.57 (p, J=6.7 Hz, 1H), 3.73 (t, J=4.8 Hz, 4H), 3.41-3.34 (m, 4H), 2.52 (s, 3H), 1.45 (d, J=6.7 Hz, 6H). LCMS (m/z): 473.2 (M+1), Rt: 1.952 min.

### 6-chloro-N-((4,5-difluoro-1H-benzo[d]imidazol-2-yl)methyl)-3-(1-isopropyl-1H-pyrazol-4-yl) imidazo[1,2-b]pyridazin-8-amine (22)

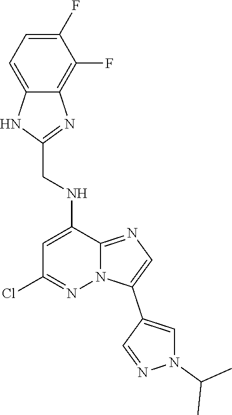 [SMILES: CC(C)n1cc(-c2cnc3c(NCc4nc5c(F)c(F)ccc5[nH]4)cc(Cl)nn23)cn1]

Synthesized according to procedure described for 4. Yield (200 mg, 46.03%). Pale-orange solid. ¹H NMR (500 MHz, DMSO) δ 8.37 (d, J=10.0 Hz, 2H). 8.07 (s, 1H), 7.85 (s, 1H), 7.27 (s, 1H), 7.24-7.19 (m, 1H), 6.32 (s, 1H), 4.87 (s, 2H), 4.64-4.59 (m, 1H), 1.47-1.45 (m, 6H). LCMS (m/z): 443.1 (M+1), Rt: 2.832 min.

### N-((4,5-difluoro-1H-benzo[d]imidazol-2-yl)methyl)-3-(1-isopropyl-1H-pyrazol-4-yl)-6-(4-methylpiperazin-1-yl) imidazo[1,2-b]pyridazin-8-amine (23)

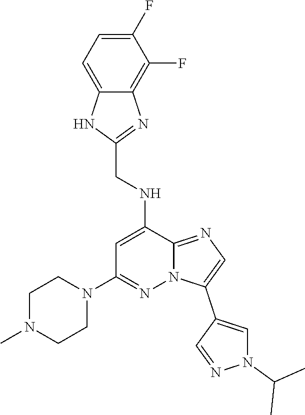 [SMILES: CC(C)n1cc(-c2cnc3c(NCc4nc5c(F)c(F)ccc5[nH]4)cc(N4CCN(C)CC4)nn23)cn1]

Synthesized according to procedure described for 5. Yield (13 mg, 18.94%), pale-brown solid. ¹H NMR (500 MHz, DMSO) δ 8.33 (s, 1H), 8.24 (s, 1H), 8.07 (s, 1H), 7.62 (s, 1H), 7.52 (t, J=6.2 Hz, 1H), 7.27 (dd, J=8.9, 3.8 Hz, 1H), 7.23-7.17 (m, 1H), 6.03 (s, 1H), 4.81 (d, J=6.1 Hz, 2H), 4.59-4.54 (m, 1H), 3.40-3.38 (m, 4H), 2.41 (1, J=5.1 Hz, 4H), 2.20 (s, 3H), 1.46 (d, J=6.6 Hz, 6H). ¹⁹F NMR (471 MHz, DMSO) δ −149.73 (d, J=170.6 Hz), −155.47 (dd, J=22.0, 7.2 Hz). LCMS (m/z): 507.2 (M+1), Rt: 1.894 min.

### N-((4,5-difluoro-1H-benzo[d]imidazol-2-yl)methyl)-3-(1-isopropyl-1H-pyrazol-4-yl)-6-morpholinoimidazo[1,2-b]pyridazin-8-amine (24)

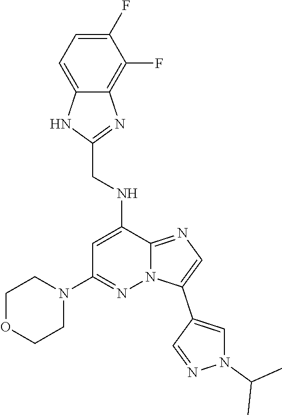 [SMILES: CC(C)n1cc(-c2cnc3c(NCc4nc5c(F)c(F)ccc5[nH]4)cc(N4CCOCC4)nn23)cn1]

Synthesized according to procedure described for 5. Yield (8 mg, 14.36%), pale-brown solid. ¹H NMR (500 MHz, DMSO) δ 12.71 (s, 1H), 8.33 (s, 1H), 8.08 (s, 1H), 7.63 (s, 1H), 7.56 (d, J=7.8 Hz, 1H), 7.31-7.12 (m, 2H), 6.03 (s, 1H), 4.82 (d, J=6.2 Hz, 2H), 4.57 (p, J=6.7 Hz, 1H), 3.72 (dd, J=5.8, 3.9 Hz, 4H), 3.37 (dd, J=6.4, 3.3 Hz, 4H), 1.46 (d, J=6.6 Hz, 6H). ¹⁹F NMR (471 MHz, DMSO) δ −150.10 (dd, J=22.2, 11.3 Hz), −155.50 (dd, J=21.6, 7.0 Hz). LCMS (m/z): 494.2 (M+1), Rt: 2.419 min.

### 6-chloro-N-((4-fluoro-1H-benzo[d]imidazol-2-yl)methyl)-3-(1-isopropyl-1H-pyrazol-4-yl) imidazo[1,2-b]pyridazin-8-amine (25)

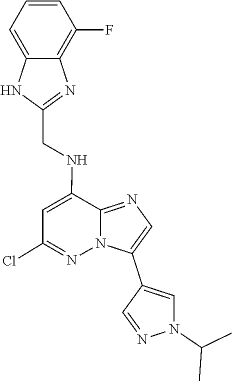 [SMILES: CC(C)n1cc(-c2cnc3c(NCc4nc5c(F)cccc5[nH]4)cc(Cl)nn23)cn1]

Synthesized according to procedure described for 4. Yield (120 mg, 28.79%). Pale-orange solid. LCMS (m/z): 425.1 (M+1), Rt: 2.689 min.

### N-((4-fluoro-1H-benzo[d]imidazol-2-yl)methyl)-3-(1-isopropyl-1H-pyrazol-4-yl)-6-(4-methylpiperazin-1-yl) imidazo[1,2-b]pyridazin-8-amine (26)

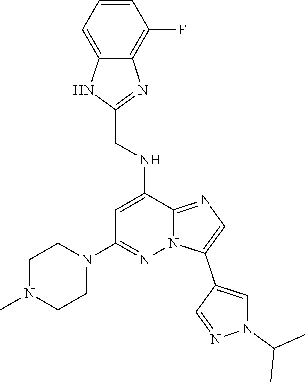 [SMILES: CC(C)n1cc(-c2cnc3c(NCc4nc5c(F)cccc5[nH]4)cc(N4CCN(C)CC4)nn23)cn1]

Synthesized according to procedure described for 5. Yield (7 mg, 12.71%), Pale-brown solid. ¹H NMR (500 MHz, DMSO) δ 12.65 (s, 1H), 8.34 (s, 1H), 8.07 (s, 1H), 7.61 (s, 1H), 7.50 (s, 1H), 7.28 (s, 1H), 7.13 (td, J=8.0, 4.8 Hz, 1H), 6.95 (t, J=9.6 Hz, 1H), 6.03 (s, 1H), 4.81 (d, J=6.1 Hz, 2H), 4.57 (p, J=6.6 Hz, 1H), 3.39 (dt, J=6.7, 3.3 Hz, 4H), 2.41 (t, J=5.0 Hz, 4H), 2.20 (s, 3H), 1.46 (d, J=6.6 Hz, 6H). ¹⁹F NMR (471 MHz, DMSO) δ −129.49 (s). LCMS (m/z): 487.2 (M−1), Rt: 1.807 min.

### N-((4-fluoro-1H-benzo[d]imidazol-2-yl)methyl)-3-(1-isopropyl-1H-pyrazol-4-yl)-6-morpholinoimidazo[1,2-b]pyridazin-8-amine (27)

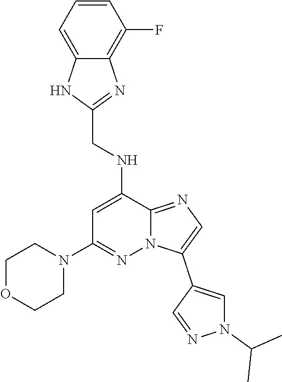 [SMILES: CC(C)n1cc(-c2cnc3c(NCc4nc5c(F)cccc5[nH]4)cc(N4CCOCC4)nn23)cn1]

Synthesized according to procedure described for 5. Yield (4 mg, 5.96%), Pale-brown solid. ¹H NMR (500 MHz, DMSO) δ 12.63 (s, 1H), 8.33 (s, 1H), 8.08 (s, 1H), 7.63 (s, 1H), 7.55 (t. J=6.2 Hz, 1H), 7.27 (d, J=8.0 Hz, 1H), 7.13 (td, J=8.0, 4.9 Hz, 1H), 6.95 (dd, J=11.1, 7.9 Hz, 1H), 6.04 (s, 1H), 4.81 (d, J=6.1 Hz, 2H), 4.57 (p, J=6.7 Hz, 1H), 3.72 (t, J=4.8 Hz, 4H), 3.38-3.35 (m, 4H), 1.46 (d, J=6.7 Hz, 6H). ¹⁹F NMR (471 MHz, DMSO) δ −129.45 (dd, J=11.2, 4.9 Hz). LCMS (m/z): 476.2 (M+1), Rt: 2.306 min.

### 6-chloro-3-(1-cyclopropyl-1H-pyrazol-4-yl)-8-ethoxyimidazo[1,2-b]pyridazine (3c)

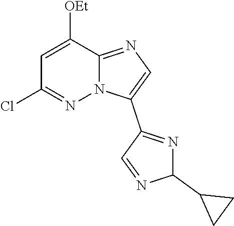 [SMILES: CCOc1cc(Cl)nn2c(C3=NC(C4CC4)N=C3)cnc12]

Following the general procedure step 3, 3-bromo-6-chloro-8-ethoxyimidazo[1,2-b]pyridazine (1.0 g, 3.62 mmol), 1-cyclopropyl-4-(4,4,5,5-tetramethyl-1,3,2-dioxaborolan-2-yl)-1H-pyrazole (0.932 g, 3.98 mmol), K₂CO₃ (1.0 g, 7.23 mmol), 1,4-dioxane (8 mL) and water (2 mL) for 10 min. To this mixture, Pd(PPh₃)₄ (209 mg) was added, and the reaction was heated to 90° C. for 16 h. Yield (0.80 g, 72.83%), Pale yellow gummy solid. ¹H NMR (500 MHz, DMSO) δ 8.43-8.40 (m, 1H), 8.07 (d, J=0.7 Hz, 1H), 7.94 (s, 1H), 6.92 (s, 1H), 4.41 (q, J=7.0 Hz, 2H), 3.92 (s, 1H), 1.45 (t, J=7.0 Hz, 3H), 1.12-1.08 (m, 2H), 1.04-1.01 (m, 2H). LCMS (m/z): 304.1 (M+1), Rt: 2.517 min.

### 6-chloro-3-(1-cyclopropyl-1H-pyrazol-4-yl)-N-((5,6-dichloro-1H-benzo[d]imidazol-2-yl)methyl) imidazo[1,2-b]pyridazin-8-amine (28)

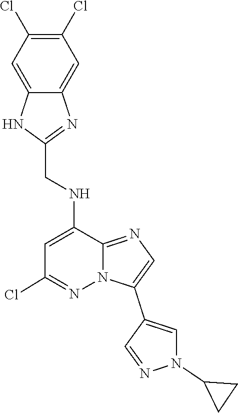 [SMILES: Clc1cc(NCc2nc3cc(Cl)c(Cl)cc3[nH]2)c2ncc(-c3cnn(C4CC4)c3)n2n1]

Synthesized according to procedure described for 4. Yield (140 mg, 44.88%). Pale-brown solid. ¹H NMR (500 MHz, DMSO) δ 8.38 (s, 1H), 8.04 (s, 1H), 7.85 (s, 1H), 7.64-7.60 (m, 1H), 7.56 (dd, J=7.4, 3.1 Hz, 1H), 6.31 (s, 1H), 1.48-1.38 (m, 1H), 1.09 (q, J=3.8 Hz, 2H), 1.01 (dt, J=7.2, 3.3 Hz, 2H). LCMS (m/z): 474.0 (M+1), Rt: 3.011 min.

### 3-(1-cyclopropyl-1H-pyrazol-4-yl)-N-((5,6-dichloro-1H-benzo[d]imidazol-2-yl)methyl)-6-(4-methylpiperazin-1-yl) imidazo[1,2-b]pyridazin-8-amine (29)

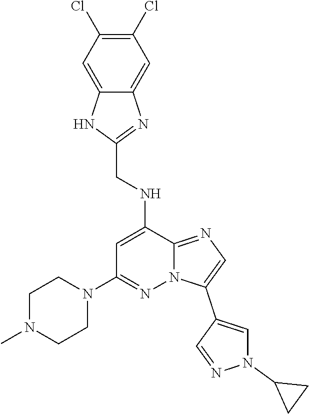 [SMILES: CN1CCN(c2cc(NCc3nc4cc(Cl)c(Cl)cc4[nH]3)c3ncc(-c4cnn(C5CC5)c4)n3n2)CC1]

Synthesized according to procedure described for 5. Yield (10 mg, 17.63%), Pale-brown solid. ¹H NMR (500 MHz, DMSO) δ 8.33 (s, 1H), 8.21 (s, 1H), 8.05 (s, 1H). 7.77 (s, 2H), 7.62 (s, 1H), 7.52 (t, J=6.2 Hz, 1H), 5.99 (s, 1H), 4.81 (d, J=6.1 Hz, 2H), 3.82 (dd, J=7.4, 3.7 Hz, 1H), 3.38 (dt, J=7.1, 2.1 Hz, 4H), 2.41 (t, J=5.0 Hz, 4H), 2.20 (s, 3H), 1.09-1.06 (m, 2H), 1.02-0.97 (m, 2H). LCMS (m/z): 538.1 (M+1), Rt: 2.091 min.

### 3-(1-cyclopropyl-1H-pyrazol-4-yl)-N-((5,6-dichloro-1H-benzo[d]imidazol-2-yl)methyl)-6-morpholinoimidazo[1,2-b]pyridazin-8-amine (30)

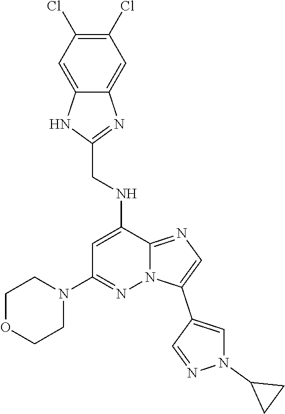 [SMILES: Clc1cc2nc(CNc3cc(N4CCOCC4)nn4c(-c5cnn(C6CC6)c5)cnc34)[nH]c2cc1Cl]

Synthesized according to procedure described for 5. Yield (5 mg, 9.03%), Pale-brown solid. ¹H NMR (500 MHz, DMSO) δ 12.62 (s, 1H), 8.34 (s, 1H), 8.05 (s, 1H), 7.77 (s, 2H), 7.63 (s, 1H), 7.56 (t, J=6.2 Hz, 1H), 6.01 (s, 1H), 4.81 (d, J=6.1 Hz, 2H), 3.82 (tt, J=7.4, 3.9 Hz, 1H), 3.72 (t, J=4.8 Hz, 4H), 3.35 (t, J=4.9 Hz, 4H), 1.12-1.04 (m, 2H), 1.04-0.94 (m, 2H). LCMS (m/z): 525.1 (M+1), Rt: 2.613 min.

### 6-chloro-N-((7-chloro-1H-benzo[d]imidazol-2-yl)methyl)-3-(1-cyclopropyl-1H-pyrazol-4-yl) imidazo[1,2-b]pyridazin-8-amine (31)

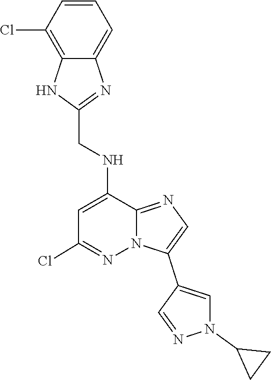 [SMILES: Clc1cc(NCc2nc3cccc(Cl)c3[nH]2)c2ncc(-c3cnn(C4CC4)c3)n2n1]

Synthesized according to procedure described for 4. Yield (140 mg, 48.40%). Pale-orange solid. ¹H NMR (500 MHz, DMSO) δ 8.38 (s, 1H), 8.08-8.00 (m, 1H), 7.83 (d, J=25.0 Hz, 1H), 7.65-7.54 (m, 1H), 7.27-7.21 (m, 1H), 7.18-7.12 (m, 1H), 6.33 (s, 1H), 4.88 (s, 2H), 1.50-1.35 (m, 1H), 1.13-1.07 (m, 2H), 1.04-0.98 (m, 2H). LCMS (m/z): 440.1 (M+1), Rt: 2.720 min.

### N-((7-chloro-1H-benzo[d]imidazol-2-yl)methyl)-3-(1-cyclopropyl-1H-pyrazol-4-yl)-6-(4-methylpiperazin-1-yl) imidazo[1,2-b]pyridazin-8-amine (32)

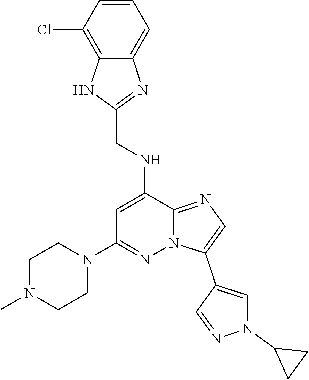 [SMILES: CN1CCN(c2cc(NCc3nc4cccc(Cl)c4[nH]3)c3ncc(-c4cnn(C5CC5)c4)n3n2)CC1]

Synthesized according to procedure described for 5. Yield (10 mg, 17.47%), Pale-brown solid. ¹H NMR (500 MHz, DMSO) δ 8.32 (d, J=9.3 Hz, 1H), 8.04 (s, 1H), 7.61 (s, 1H), 7.52 (s, 1H), 7.44 (d, J=8.0 Hz, 1H), 7.22 (dd, J=7.8, 1.0 Hz, 1H), 7.14 (t, J=7.9 Hz, 1H), 6.09 (s, 1H), 4.81 (d, J=5.9 Hz, 2H), 3.81 (dt, J=7.4, 3.6 Hz, 1H), 3.39 (dt, J=7.2, 3.4 Hz, 4H), 2.41 (t, J=5.0 Hz, 4H), 2.20 (s, 3H), 1.09-1.04 (m, 2H), 1.02-0.97 (m, 2H). LCMS (m/z): 504.2 (M+1), Rt: 1.856 min.

### N-((7-chloro-1H-benzo[d]imidazol-2-yl)methyl)-3-(1-cyclopropyl-1H-pyrazol-4-yl)-6-morpholinoimidazo[1,2-b]pyridazin-8-amine (33)

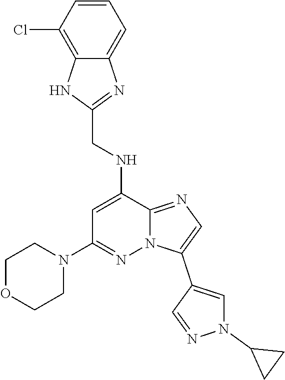 [SMILES: Clc1cccc2nc(CNc3cc(N4CCOCC4)nn4c(-c5cnn(C6CC6)c5)cnc34)[nH]c12]

Synthesized according to procedure described for 5. Yield (5 mg, 89.97%), Pale-brown solid. ¹H NMR (500 MHz, DMSO) δ 12.67 (s, 1H), 8.33 (s, 1H), 8.05 (s, 1H), 7.63 (s, 1H), 7.59 (s, 1H), 7.41 (d, J=8.7 Hz, 1H), 7.22 (d, J=7.7 Hz, 1H), 7.15 (t, J=7.9 Hz, 1H), 6.10 (s, 1H), 4.82 (d, J=6.1 Hz, 2H), 3.81 (dd, J=7.5, 3.7 Hz, 1H), 3.72 (t, J=4.9 Hz, 4H), 3.37 (t, J=4.8 Hz, 4H), 1.11-1.05 (m, 2H), 1.02-0.95 (m, 2H). LCMS (m/z): 490.1 (M+1), Rt: 2.354 min.

### 6-chloro-3-(1-cyclopropyl-1H-pyrazol-4-yl)-N-((4-fluoro-1H-benzo[d]imidazol-2-yl)methyl) imidazo[1,2-b]pyridazin-8-amine (34)

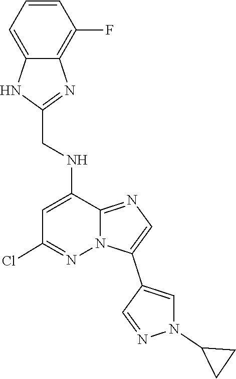 [SMILES: Fc1cccc2[nH]c(CNc3cc(Cl)nn4c(-c5cnn(C6CC6)c5)cnc34)nc12]

Synthesized according to procedure described for 4. Yield (135 mg, 48.49%). Pale-orange solid. ¹H NMR (500 MHz, DMSO) δ 8.38 (s, 1H), 8.04 (dd, J=5.6, 0.7 Hz, 1H), 7.85 (s, 1H), 7.65-7.53 (m, 1H), 7.29 (s, 1H), 7.14 (td, J=8.0, 4.8 Hz, 1H), 6.32 (s, 1H), 4.86 (s, 2H), 3.85 (tt, J=7.4, 3.9 Hz, 1H), 1.10-1.08 (m, 2H), 1.01 (dd, J=7.0, 1.1 Hz, 2H). ¹⁹F NMR (471 MHz, DMSO) δ −129.22 (d, J=11.4 Hz). LCMS (m/z): 423.0 (M+1), Rt: 2.607 min.

### 3-(1-cyclopropyl-1H-pyrazol-4-yl)-N-((4-fluoro-1H-benzo[d]imidazol-2-yl)methyl)-6-(4-methylpiperazin-1-yl) imidazo[1,2-b]pyridazin-8-amine (35)

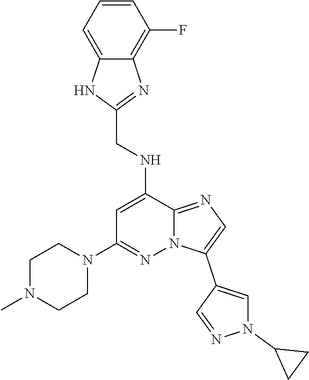 [SMILES: CN1CCN(c2cc(NCc3nc4c(F)cccc4[nH]3)c3ncc(-c4cnn(C5CC5)c4)n3n2)CC1]

Synthesized according to procedure described for 5. Yield (15 mg, 26.07%), Pale-brown solid. ¹H NMR (500 MHz, DMSO) δ 12.64 (s, 1H), 8.33 (s, 1H), 8.18 (s, 1H), 8.05 (d, J=0.7 Hz, 1H), 7.62 (s, 1H), 7.50 (s, 1H), 7.29 (d, J=8.1 Hz, 1H), 7.13 (td, J=8.0, 4.9 Hz, 1H), 6.96 (dd, J=11.1, 8.0 Hz, 1H), 6.04 (s, 1H), 4.81 (d, J=5.9 Hz, 2H), 3.82 (dd, J=7.3, 3.7 Hz, 1H), 3.40-3.37 (m, 4H), 2.42 (t, J=5.0 Hz, 4H), 2.21 (s, 3H), 1.09-1.05 (m, 2H), 1.03-0.95 (m, 2H). ¹⁹F NMR (471 MHz, DMSO) δ −129.54. LCMS (m/z): 487.2 (M+1), Rt: 1.775 min.

### 3-(1-cyclopropyl-1H-pyrazol-4-yl)-N-((4-fluoro-1H-benzo[d]imidazol-2-yl)methyl)-6-morpholinoimidazo[1,2-b]pyridazin-8-amine (36)

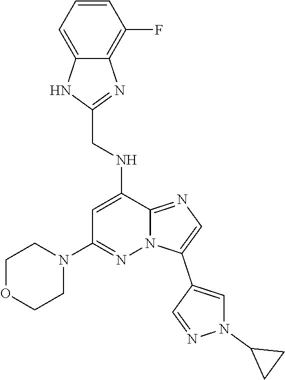 [SMILES: Fc1cccc2[nH]c(CNc3cc(N4CCOCC4)nn4c(-c5cnn(C6CC6)c5)cnc34)nc12]

Synthesized according to procedure described for 5. Yield (7 mg, 12.50%), Pale-brown solid. ¹H NMR (500 MHz, DMSO) δ 12.61 (s, 1H), 8.34 (s, 1H), 8.05 (s, 1H), 7.63 (s, 1H), 7.56 (t, J=6.1 Hz, 1H), 7.27 (d, J=8.0 Hz, 1H), 7.13 (td, J=8.0, 4.8 Hz, 1H), 6.94 (dd, J=11.1, 7.9 Hz, 1H), 6.04 (s, 1H), 4.81 (d, J=6.1 Hz, 2H), 3.82 (tt, J=7.4, 3.8 Hz, 1H), 3.76-3.64 (m, 4H), 3.37 (q, J=6.1, 4.8 Hz, 4H), 1.10-1.06 (m, 2H), 1.04-0.95 (m, 2H). ¹⁹F NMR (471 MHz, DMSO) δ −129.60 (s). LCMS (m/z): 474.2 (M+1), Rt: 2.228 min.

### 6-chloro-3-(1-cyclopropyl-1H-pyrazol-4-yl)-N-((4,5-difluoro-1H-benzo[d]imidazol-2-yl)methyl) imidazo[1,2-b]pyridazin-8-amine (37)

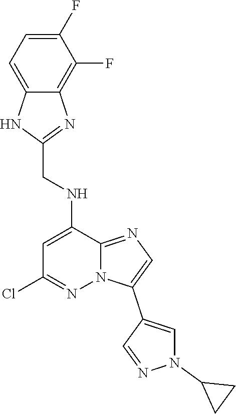 [SMILES: Fc1ccc2[nH]c(CNc3cc(Cl)nn4c(-c5cnn(C6CC6)c5)cnc34)nc2c1F]

Synthesized according to procedure described for 4. Yield (150 mg, 51.68%). Pale-orange solid. ¹H NMR (500 MHz, DMSO) δ 8.39 (td, J=6.8, 4.9 Hz, 2H), 8.04 (dd, J=5.8, 0.8 Hz, 1H), 7.86 (s, 1H), 7.67-7.49 (m, 1H), 7.30-7.24 (m, 1H), 7.23-7.18 (m, 1H), 6.33 (s, 1H), 4.87 (s, 2H), 3.85 (tt, J=7.4, 3.9 Hz, 1H), 1.11-1.08 (m, 2H), 1.02-0.99 (m, 2H). ¹⁹F NMR (471 MHz, DMSO) δ −149.94, −155.25 (d, J=20.9 Hz). LCMS (m/z): 441.1 (M+1), Rt: 2.748 min.

### 3-(1-cyclopropyl-1H-pyrazol-4-yl)-N-((4,5-difluoro-1H-benzo[d]imidazol-2-yl)methyl)-6-(4-methylpiperazin-1-yl) imidazo[1,2-b]pyridazin-8-amine (38)

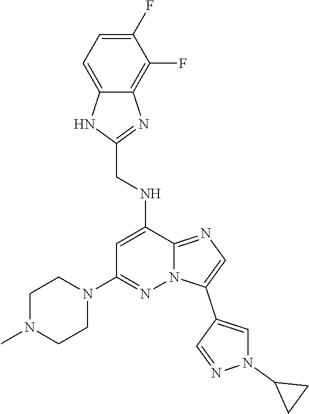 [SMILES: CN1CCN(c2cc(NCc3nc4c(F)c(F)ccc4[nH]3)c3ncc(-c4cnn(C5CC5)c4)n3n2)CC1]

Synthesized according to procedure described for 5. Yield (10 mg, 17.47%), Pale-brown solid. ¹H NMR (500 MHz, DMSO) δ 12.72 (s, 1H), 8.33 (s, 1H), 8.17 (s, 1H), 8.05 (s, 1H), 7.62 (s, 1H), 7.52 (t, J=6.2 Hz, 1H), 7.26 (s, 1H), 7.22-7.18 (m, 1H), 6.03 (s, 1H), 4.81 (d, J=6.1 Hz, 2H), 3.81 (dt, J=7.3, 3.6 Hz, 1H), 3.41-3.37 (m, 4H), 2.42 (t, J=5.0 Hz, 4H), 2.21 (s, 3H), 1.08 (dd, J=5.2, 2.7 Hz, 2H), 1.02-0.97 (m, 2H). ¹⁹F NMR (471 MHz, DMSO) δ −150.76, −155.51. LCMS (m/z): 505.2 (M+1), Rt: 1.865 min.

### 3-(1-cyclopropyl-1H-pyrazol-4-yl)-N-((4,5-difluoro-1H-benzo[d]imidazol-2-yl)methyl)-6-morpholinoimidazo[1,2-b]pyridazin-8-amine (39)

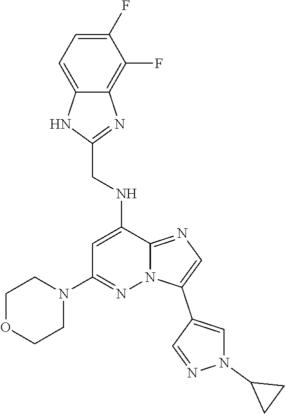 [SMILES: Fc1ccc2[nH]c(CNc3cc(N4CCOCC4)nn4c(-c5cnn(C6CC6)c5)cnc34)nc2c1F]

Synthesized according to procedure described for 5. Yield (2.30 mg, 4.13%), Off-white solid. ¹H NMR (500 MHz, DMSO) δ 12.85 (s, 1H), 8.34 (s, 1H), 8.05 (s, 1H), 7.63 (s, 1H), 7.56 (t, J=6.2 Hz, 1H), 7.26 (dd, J=8.9, 3.7 Hz, 1H), 7.21-7.16 (m, 1H), 6.04 (s, 1H), 4.81 (d, J=6.0 Hz, 2H), 3.82 (tt, J=7.4, 3.9 Hz, 1H), 3.72 (t, J=4.7 Hz, 4H), 3.37 (t, J=4.7 Hz, 4H), 1.08 (p, J=4.3 Hz, 2H), 0.99 (td, J=7.4, 4.8 Hz, 2H). ¹⁹F NMR (471 MHz, DMSO) δ −150.14, −155.50. LCMS (m/z): 492.1 (M+1), Rt: 2.357 min.

### 6-chloro-3-(1-(difluoromethyl)-1H-pyrazol-4-yl)-8-ethoxyimidazo[1,2-b]pyridazine (3d)

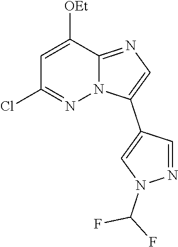 [SMILES: CCOc1cc(Cl)nn2c(-c3cnn(C(F)F)c3)cnc12]

Following the general procedure step 3, 3-bromo-6-chloro-8-ethoxyimidazo[1,2-b]pyridazine (2.0 g, 7.23 mmol), 1-(difluoro methyl)-4-(4,4,5,5-tetramethyl-1,3,2-dioxaborolan-2-yl)-1H-pyrazole (2.0 g, 7.96 mmol), K₂CO₃ (1.0 g, 14.47 mmol), 1,4-dioxane (16 mL) and water (4 mL) for 10 min. To this mixture, Pd(PPh₃)₄ (835 mg) was added, and the reaction was heated to 90° C. for 16 h. Yield (1.30 g, 57.30%), white solid. LCMS (m/z): 314.0 (M+1), Rt: 2.634 min.

### 6-chloro-N-((5,6-dichloro-1H-benzo[d]imidazol-2-yl)methyl)-3-(1-(difluoromethyl)-1H-pyrazol-4-yl) imidazo[1,2-b]pyridazin-8-amine (40)

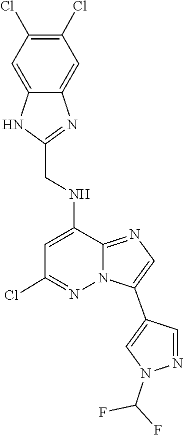 [SMILES: FC(F)n1cc(-c2cnc3c(NCc4nc5cc(Cl)c(Cl)cc5[nH]4)cc(Cl)nn23)cn1]

Synthesized according to procedure described for 4. Yield (150 mg, 34.62%). Pale-brown solid. LCMS (m/z): 485.0 (M+1), Rt: 3.082 min.

### N-((5,6-dichloro-1H-benzo[d]imidazol-2-yl)methyl)-3-(1-(difluoromethyl)-1H-pyrazol-4-yl)-6-(4-methylpiperazin-1-yl) imidazo[1,2-b]pyridazin-8-amine (41)

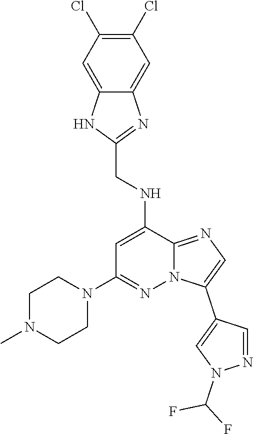 [SMILES: CN1CCN(c2cc(NCc3nc4cc(Cl)c(Cl)cc4[nH]3)c3ncc(-c4cnn(C(F)F)c4)n3n2)CC1]

Synthesized according to procedure described for 5. Yield (4 mg, 7.07%), Pale-yellow solid. ¹H NMR (500 MHz, DMSO) δ 8.74 (s, 1H), 8.43 (s, 1H), 8.25 (s, 1H), 7.93 (s, 1H), 7.80 (s, 1H), 7.77 (s, 1H), 7.61 (t, J=6.2 Hz, 1H), 6.05 (s, 1H), 4.82 (d, J=6.2 Hz, 2H), 3.39 (dd, J=6.5, 3.5 Hz, 5H), 2.41 (t, J=5.0 Hz, 4H), 2.20 (s, 3H). LCMS (m/z): 548.1 (M+1), Rt: 2.170 min.

### N-((5,6-dichloro-1H-benzo[d]imidazol-2-yl)methyl)-3-(1-(difluoromethyl)-1H-pyrazol-4-yl)-6-morpholinoimidazo[1,2-b]pyridazin-8-amine (42)

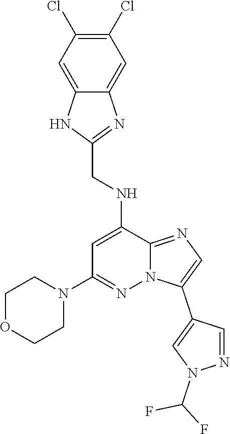 [SMILES: FC(F)n1cc(-c2cnc3c(NCc4nc5cc(Cl)c(Cl)cc5[nH]4)cc(N4CCOCC4)nn23)cn1]

Synthesized according to procedure described for 5. Yield (4 mg, 7.24%), Pale-yellow solid. ¹H NMR (500 MHz, DMSO) δ 8.75 (s, 1H), 8.44 (s, 1H), 7.93 (s, 1H), 7.81 (s, 2H), 7.68-7.57 (m, 2H), 6.06 (s, 1H), 4.83 (d, J=6.1 Hz, 2H), 3.72 (t, J=4.7 Hz, 4H), 3.37 (t, J=4.8 Hz, 4H). LCMS (m/z): 535.1 (M+1), Rt: 2.774 min.

### N-((5,6-dichloro-1H-benzo[d]imidazol-2-yl)methyl)-3-(1-(difluoromethyl)-1H-pyrazol-4-yl)-6-(piperazin-1-yl) imidazo[1,2-b]pyridazin-8-amine (43)

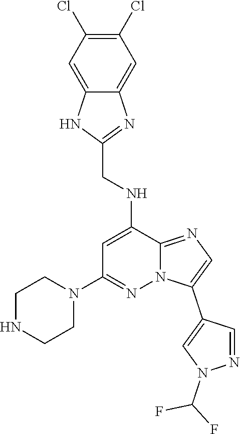 [SMILES: FC(F)n1cc(-c2cnc3c(NCc4nc5cc(Cl)c(Cl)cc5[nH]4)cc(N4CCNCC4)nn23)cn1]

Synthesized according to procedure described for 5. Yield (9 mg, 26.72%), Off-white solid. ¹H NMR (500 MHz, DMSO) δ 8.74 (s, 1H), 8.44 (s, 1H), 8.23 (s, 1H), 7.93 (s, 1H), 7.80 (s, 1H), 7.77 (s, 2H), 7.63 (t, J=6.2 Hz, 1H), 6.04 (s, 1H), 4.82 (d, J=6.2 Hz, 2H), 3.37 (d, J=10.1 Hz, 4H), 2.90-2.85 (m, 4H), ¹⁹F NMR (471 MHz, DMSO) δ −94.22, −94.34, LCMS (m/z): 534.1 (M+1), Rt: 2.152 min.

### 6-(3-aminopyrrolidin-1-yl)-N-((5,6-dichloro-1H-benzo[d]imidazol-2-yl)methyl)-3-(1-(difluoromethyl)-1H-pyrazol-4-yl) imidazo[1,2-b]pyridazin-8-amine (44)

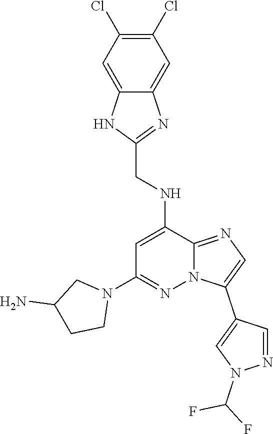 [SMILES: NC1CCN(c2cc(NCc3nc4cc(Cl)c(Cl)cc4[nH]3)c3ncc(-c4cnn(C(F)F)c4)n3n2)C1]

Synthesized according to procedure described for 5. Yield (10 mg, 29.69%), Off-white solid. ¹H NMR (500 MHz, DMSO) δ 8.78 (d, J=3.2 Hz, 1H), 8.46 (s, 1H), 8.34 (s, 1H), 7.95 (d, J=8.4 Hz, 1H), 7.77 (s, 2H), 7.76 (s, 1H), 7.61 (t, J=6.1 Hz, 1H), 5.63 (s, 1H), 4.80 (d, J=6.0 Hz, 2H), 3.67 (q, J=5.4 Hz, 1H), 3.57-3.51 (m, 2H), 3.39 (ddd, J=7.0, 5.2, 3.7 Hz, 2H), 3.20 (dd, J=10.6, 4.1 Hz, 2H), 2.15-2.09 (m, 1H), 1.81 (ddd, J=12.5, 7.4, 3.8 Hz, 1H), ¹⁹F NMR (471 MHz, DMSO) δ −94.09, −94.21, LCMS (m/z): 534.1 (M+1), Rt: 2.140 min.

### 6-(4-aminopiperidin-1-yl)-N-((5,6-dichloro-1H-benzo[d]imidazol-2-yl)methyl)-3-(1-(difluoromethyl)-1H-pyrazol-4-yl) imidazo[1,2-b]pyridazin-8-amine (45)

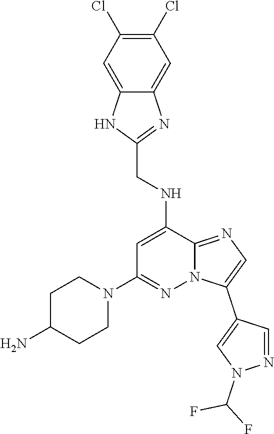 [SMILES: NC1CCN(c2cc(NCc3nc4cc(Cl)c(Cl)cc4[nH]3)c3ncc(-c4cnn(C(F)F)c4)n3n2)CC1]

Synthesized according to procedure described for 5. Yield (5.50 mg, 22.69%), Off-white solid. ¹H NMR (500 MHz, DMSO) δ 8.73 (d, J=1.4 Hz, 1H), 8.43 (d, J=1.6 Hz, 1H), 8.41 (s, 1H), 7.79 (d, J=2.3 Hz, 1H), 7.77 (s, 2H), 7.64-7.59 (m, 1H), 6.05 (d, J=4.3 Hz, 1H), 4.82 (d, J=6.1 Hz, 2H), 4.03 (d, J=12.8 Hz, 2H), 3.92-3.86 (m, 1H), 2.92 (t, J=12.4 Hz, 2H), 2.66 (s, 1H), 1.90-1.80 (m, 2H), 1.39 (dd, J=16.8, 6.5 Hz, 2H), ¹⁹F NMR (471 MHz, DMSO) δ −94.23, −94.36, LCMS (m/z): 546.1 (M−1), Rt: 2.188 min.

### N8-((5,6-dichloro-1H-benzo[d]imidazol-2-yl)methyl)-3-(1-(difluoromethyl)-1H-pyrazol-4-yl)-N6-(1-methylpiperidin-4-yl) imidazo[1,2-b]pyridazine-6,8-diamine (46)

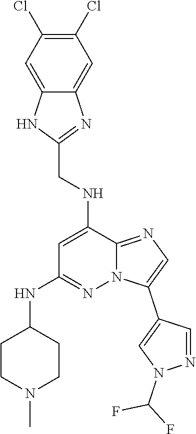 [SMILES: CN1CCC(Nc2cc(NCc3nc4cc(Cl)c(Cl)cc4[nH]3)c3ncc(-c4cnn(C(F)F)c4)n3n2)CC1]

Synthesized according to procedure described for 5. Yield (6 mg, 12.92%), Off-white solid. ¹H NMR (500 MHz, DMSO) δ 8.74 (s, 1H), 8.43 (s, 1H), 8.25 (s, 1H), 7.93 (s, 1H), 7.80 (s, 1H), 7.77 (s, 1H), 7.61 (t, J=6.2 Hz, 1H), 6.05 (s, 1H), 4.82 (d, J=6.2 Hz, 2H), 3.39 (dd, J=6.5, 3.5 Hz, 5H), 2.41 (t, J=5.0 Hz, 4H), 2.20 (s, 3H), LCMS (m/z): 560.2 (M−1), Rt: 2.149 min.

### N-((5,6-dichloro-1H-benzo[d]imidazol-2-yl)methyl)-3-(1-(difluoromethyl)-1H-pyrazol-4-yl)-6-(3,3-dimethylpiperazin-1-yl) imidazo[1,2-b]pyridazin-8-amine (47)

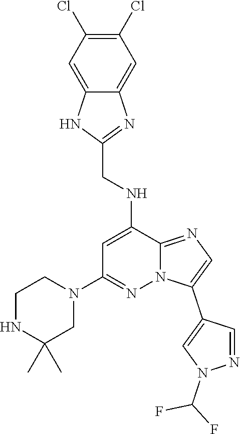 [SMILES: CC1(C)CN(c2cc(NCc3nc4cc(Cl)c(Cl)cc4[nH]3)c3ncc(-c4cnn(C(F)F)c4)n3n2)CCN1]

Synthesized according to procedure described for 5. Yield (5.30 mg, 20.82%), Off-white solid. ¹H NMR (500 MHz, DMSO) δ 8.73 (s, 1H), 8.43 (s, 1H), 8.25 (s, 1H), 7.79 (s, 1H), 7.77 (s, 1H), 7.63 (t, J=6.1 Hz, 1H), 5.95 (s, 1H), 4.82 (d, J=6.1 Hz, 2H), 3.43-3.40 (m, 2H), 3.15 (s, 2H), 2.94-2.89 (m, 2H), 1.04 (s, 6H), ¹⁹F NMR (471 MHz, DMSO) δ −94.25, −94.38, LCMS (m/z): 560.2 (M−1), Rt: 2.215 min.

### N-((5,6-dichloro-1H-benzo[d]imidazol-2-yl)methyl)-3-(1-(difluoromethyl)-1H-pyrazol-4-yl)-6-(2,2-dimethylmorpholino) imidazo[1,2-b]pyridazin-8-amine (48)

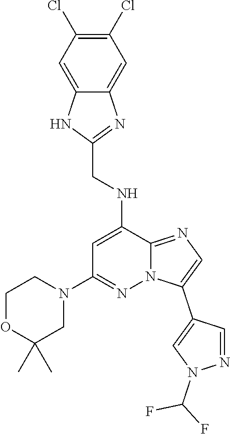 [SMILES: CC1(C)CN(c2cc(NCc3nc4cc(Cl)c(Cl)cc4[nH]3)c3ncc(-c4cnn(C(F)F)c4)n3n2)CCO1]

Synthesized according to procedure described for 5. Yield (7 mg, 28.82%), Off-white solid. ¹H NMR (500 MHz, DMSO) δ 12.64 (s, 1H), 8.73 (s, 1H), 8.43 (s, 1H), 7.85 (s, 1H), 7.79 (s, 1H), 7.69 (s, 1H), 7.64 (t, J=6.1 Hz, 1H), 5.99 (s, 1H), 4.83 (d, J=6.1 Hz, 2H), 3.72 (dd, J=6.1, 4.0 Hz, 2H), 3.40-3.37 (m, 2H), 3.18 (s, 2H), 1.14 (s, 6H), ¹⁹F NMR (471 MHz, DMSO) δ −94.26, −94.39, LCMS (m/z): 561.1 (M−1), Rt: 2.949 min.

### N-((5,6-dichloro-1H-benzo[d]imidazol-2-yl)methyl)-3-(1-(difluoromethyl)-1H-pyrazol-4-yl)-6-(3,3,4-trimethylpiperazin-1-yl) imidazo[1,2-b]pyridazin-8-amine (49)

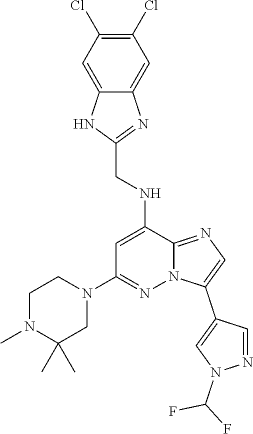 [SMILES: CN1CCN(c2cc(NCc3nc4cc(Cl)c(Cl)cc4[nH]3)c3ncc(-c4cnn(C(F)F)c4)n3n2)CC1(C)C]

Synthesized according to procedure described for 5. Yield (4 mg, 11.82%), Off-white solid. ¹H NMR (500 MHz, DMSO) δ 12.49 (s, 1H), 8.82 (s, 1H), 8.45 (s, 1H), 8.30 (s, 1H), 8.23 (s, 1H), 8.16 (s, 1H), 7.99 (d, J=58.8 Hz, 1H), 7.69 (s, 2H), 4.76 (s, 2H), 3.62 (s, 2H), 2.33 (s, 2H), 2.07 (s, 3H), 0.67 (s, 6H), ¹⁹F NMR (471 MHz, DMSO) δ −94.46, −94.59, LCMS (m/z): 571.1 (M−1), Rt: 2.139 min.

### 6-chloro-3-(1-(difluoromethyl)-1H-pyrazol-4-yl)-N-((4-fluoro-1H-benzo[d]imidazol-2-yl)methyl) imidazo[1,2-b]pyridazin-8-amine (50)

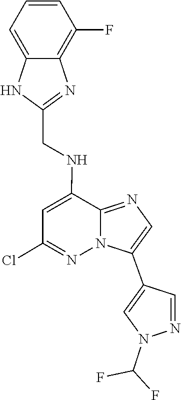 [SMILES: Fc1cccc2[nH]c(CNc3cc(Cl)nn4c(-c5cnn(C(F)F)c5)cnc34)nc12]

Synthesized according to procedure described for 4. Yield (100 mg, 41.79%). Pale-brown solid. LCMS (m/z): 433.0 (M+1), Rt: 2.675 min.

### 3-(1-(difluoromethyl)-1H-pyrazol-4-yl)-N-((4-fluoro-1H-benzo[d]imidazol-2-yl)methyl)-6-(4-methylpiperazin-1-yl) imidazo[1,2-b]pyridazin-8-amine (51)

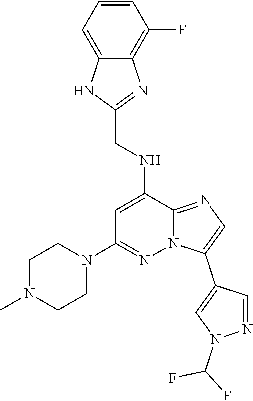 [SMILES: CN1CCN(c2cc(NCc3nc4c(F)cccc4[nH]3)c3ncc(-c4cnn(C(F)F)c4)n3n2)CC1]

Synthesized according to procedure described for 5. Yield (2.60 mg, 4.53%), Pale-yellow solid. ¹H NMR (500 MHz, DMSO) δ 8.74 (s, 1H), 8.43 (s, 1H), 8.28 (s, 2H), 7.93 (s, 1H), 7.81 (d, J=6.5 Hz, 1H), 7.13 (dt, J=8.0, 4.0 Hz, 1H), 6.09 (s, 1H), 4.82 (d, J=6.1 Hz, 2H), 3.56-3.47 (m, 4H), 3.40 (s, 5H), 2.41 (t, J=5.0 Hz, 3H). LCMS (m/z): 497.1 (M+1), Rt: 1.802 min.

### 3-(1-(difluoromethyl)-1H-pyrazol-4-yl)-N-((4-fluoro-1H-benzo[d]imidazol-2-yl)methyl)-6-morpholinoimidazo[1,2-b]pyridazin-8-amine (52)

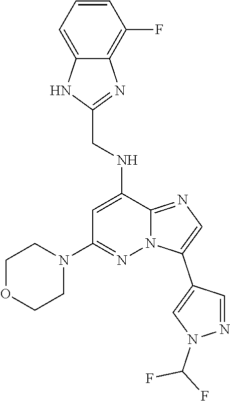 [SMILES: Fc1cccc2[nH]c(CNc3cc(N4CCOCC4)nn4c(-c5cnn(C(F)F)c5)cnc34)nc12]

Synthesized according to procedure described for 5. Yield (2.80 mg, 5.01%), Pale-yellow solid. ¹H NMR (500 MHz, CDCl₃) δ 8.34 (s, 1H), 8.02 (s, 2H), 7.39 (s, 1H), 7.13-7.07 (m, 1H), 6.89 (t, J=9.3 Hz, 1H), 6.15 (s, 1H), 4.99 (d, J=5.7 Hz, 2H), 3.90-3.85 (m, 1H), 3.80 (t, J=4.9 Hz, 4H), 3.39 (t, J=4.9 Hz, 4H). LCMS (m/z): 484.1 (M+1), Rt: 2.352 min.

### 6-chloro-N-((4,5-difluoro-1H-benzo[d]imidazol-2-yl)methyl)-3-(1-(difluoromethyl)-1H-pyrazol-4-yl) imidazo[1,2-b]pyridazin-8-amine (53)

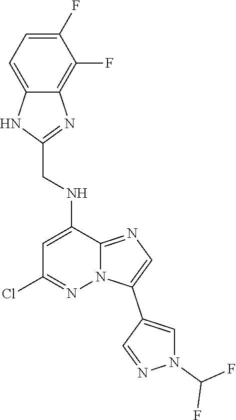 [SMILES: Fc1ccc2[nH]c(CNc3cc(Cl)nn4c(-c5cnn(C(F)F)c5)cnc34)nc2c1F]

Synthesized according to procedure described for 4. Yield (100 mg, 77.32%). Pale-brown solid. LCMS (m/z): 451.0 (M+1), Rt: 2.819 min.

### N-((4,5-difluoro-1H-benzo[d]imidazol-2-yl)methyl)-3-(1-(difluoromethyl)-1H-pyrazol-4-yl)-6-(4-methylpiperazin-1-yl) imidazo[1,2-b]pyridazin-8-amine (54)

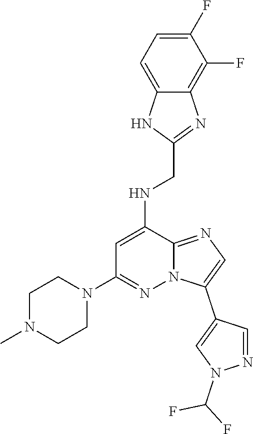 [SMILES: CN1CCN(c2cc(NCc3nc4c(F)c(F)ccc4[nH]3)c3ncc(-c4cnn(C(F)F)c4)n3n2)CC1]

Synthesized according to procedure described for 5. Yield (5 mg, 10.95%), Pale-yellow solid. ¹H NMR (500 MHz, DMSO) δ 8.74 (s, 1H), 8.44 (s, 1H), 7.93 (s, 1H), 7.81 (d, J=6.7 Hz, 1H), 7.62 (t, J=6.2 Hz, 1H), 7.27 (dd, J=8.7, 3.8 Hz, 1H), 7.23-7.17 (m, 1H), 6.08 (s, 1H), 4.83 (d, J=6.0 Hz, 2H), 3.40 (d, J=10.2 Hz, 4H), 2.81 (q, J=6.6, 5.0 Hz, 1H), 2.41 (t, J=5.0 Hz, 4H), 2.20 (s, 3H), ¹⁹F NMR (471 MHz, DMSO) δ −94.24 (d, J=26.9 Hz), −94.34-−94.41 (m), −155.28-−155.82 (m), LCMS (m/z): 513.2 (M−1), Rt: 1.963 min.

### N-((4,5-difluoro-1H-benzo[d]imidazol-2-yl)methyl)-3-(1-(difluoromethyl)-1H-pyrazol-4-yl)-6-morpholinoimidazo[1,2-b]pyridazin-8-amine (55)

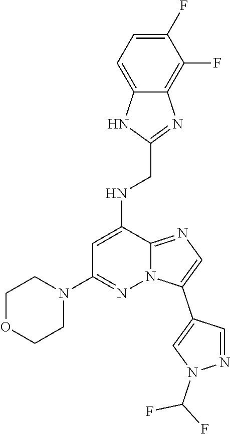 [SMILES: Fc1ccc2[nH]c(CNc3cc(N4CCOCC4)nn4c(-c5cnn(C(F)F)c5)cnc34)nc2c1F]

Synthesized according to procedure described for 5. Yield (5 mg, 11.24%), Off-white solid. ¹H NMR (500 MHz, DMSO) δ 8.75 (s, 1H), 8.44 (s, 1H), 7.81 (s, 1H), 7.69-7.60 (m, 2H), 7.55 (td, J=7.4, 3.0 Hz, 1H), 7.27 (dd, J=8.9, 3.8 Hz, 1H), 7.19 (dt, J=11.5, 7.9 Hz, 1H), 6.09 (s, 1H), 4.83 (d, J=6.1 Hz, 2H), 3.78-3.69 (m, 4H), 3.38 (t, J=4.7 Hz, 4H), 19F NMR (471 MHz, DMSO) δ −94.22, −94.34, −155.22-−155.83 (m), LCMS (m/z): 502.1 (M+1), Rt: 2.521 min.

### 6-chloro-3-(1-(difluoromethyl)-1H-pyrazol-4-yl)-N-((7-methyl-3H-imidazo[4,5-b]pyridin-2-yl)methyl) imidazo[1,2-b]pyridazin-8-amine (56)

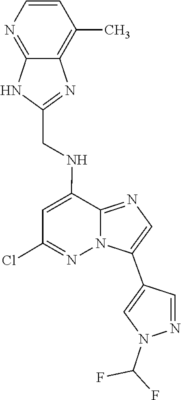 [SMILES: Cc1ccnc2[nH]c(CNc3cc(Cl)nn4c(-c5cnn(C(F)F)c5)cnc34)nc12]

Synthesized according to procedure described for 5. Yield (100 mg, 81.24%), Off-white solid. LCMS (m/z): 430.0 (M+1), Rt: 2.180 min.

### 3-(1-(difluoromethyl)-1H-pyrazol-4-yl)-N-((7-methyl-3H-imidazo[4,5-b]pyridin-2-yl)methyl)-6-(4-methylpiperazin-1-yl) imidazo[1,2-b]pyridazin-8-amine (57)

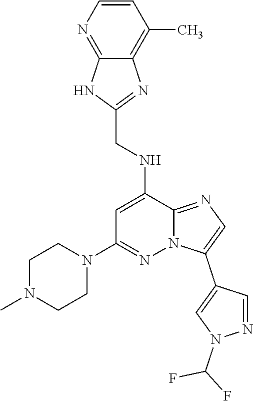 [SMILES: Cc1ccnc2[nH]c(CNc3cc(N4CCN(C)CC4)nn4c(-c5cnn(C(F)F)c5)cnc34)nc12]

Synthesized according to procedure described for 5. Yield (10 mg, 21.77%), Off-white solid. ¹H NMR (500 MHz, DMSO) δ 12.92 (s, 1H), 8.74 (s, 1H), 8.43 (s, 1H), 8.14 (s, 1H), 7.99 (d, J=59.0 Hz, 1H), 7.79 (s, 1H), 7.53 (t, J=6.0 Hz, 1H), 7.02 (d, J=4.9 Hz, 1H), 6.12 (s, 1H), 4.80 (d, J=5.9 Hz, 2H), 3.40 (dd, J=6.3, 3.8 Hz, 4H), 2.52 (s, 3H), 2.42 (t, J=5.0 Hz, 4H), 2.21 (s, 3H), ¹⁹F NMR (471 MHz, DMSO) δ −94.24, −94.36, LCMS (m/z): 494.2 (M+1), Rt: 1.628 min.

### 3-(1-(difluoromethyl)-1H-pyrazol-4-yl)-N-((7-methyl-3H-imidazo[4,5-b]pyridin-2-yl)methyl)-6-morpholinoimidazo[1,2-b]pyridazin-8-amine (58)

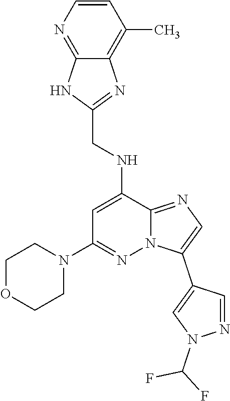 [SMILES: Cc1ccnc2[nH]c(CNc3cc(N4CCOCC4)nn4c(-c5cnn(C(F)F)c5)cnc34)nc12]

Synthesized according to procedure described for 5. Yield (13 mg, 29.07%), Off-white solid. ¹H NMR (500 MHz, DMSO) δ 12.85 (d, J=71.6 Hz, 1H), 8.74 (s, 1H), 8.44 (s, 1H), 8.13 (s, 1H), 7.98 (d, J=58.9 Hz, 1H), 7.81 (s, 1H), 7.57 (t, J=6.0 Hz, 1H), 7.02 (d, J=4.8 Hz, 1H), 6.13 (s, 1H), 4.81 (s, 2H), 3.73 (dd, J=5.8, 3.8 Hz, 4H), 3.42-3.35 (m, 4H), 2.52 (s, 3H), ¹⁹F NMR (471 MHz, DMSO) δ −94.22, −94.35, LCMS (m/z): 481.1 (M+1), Rt: 2.012 min.

### 6-chloro-N-((5,6-dichloro-1H-benzo[d]imidazol-2-yl)methyl)-3-(1H-pyrazol-4-yl) imidazo[1,2-b]pyridazin-8-amine (59)

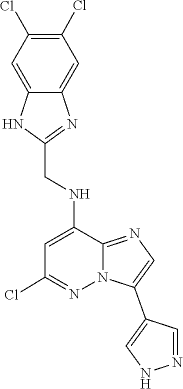 [SMILES: Clc1cc(NCc2nc3cc(Cl)c(Cl)cc3[nH]2)c2ncc(-c3cn[nH]c3)n2n1]

Synthesized according to procedure described for 4. Yield (200 mg, 40.53%). Pale-yellow solid. LCMS (m/z): 434.9 (M+1), Rt: 2.595 min.

### N-((5,6-dichloro-1H-benzo[d]imidazol-2-yl)methyl)-6-(4-methylpiperazin-1-yl)-3-(1H-pyrazol-4-yl) imidazo[1,2-b]pyridazin-8-amine (60)

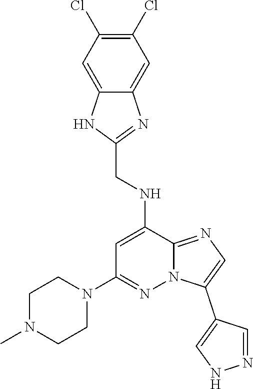 [SMILES: CN1CCN(c2cc(NCc3nc4cc(Cl)c(Cl)cc4[nH]3)c3ncc(-c4cn[nH]c4)n3n2)CC1]

Synthesized according to procedure described for 5. Yield (15 mg, 26.16%), pale-yellow solid. ¹H NMR (500 MHz, DMSO) δ 8.22 (s, 2H), 8.16 (s, 1H), 7.77 (s, 2H), 7.64 (s, 1H), 7.52 (t, J=6.2 Hz, 1H), 6.00 (s, 1H), 4.81 (d, J=6.2 Hz, 2H), 3.38 (t, J=4.9 Hz, 4H), 3.17 (s, 3H), 2.42 (t, J=5.0 Hz, 4H). LCMS (m/z): 498.1 (M+1), Rt: 1.879 min.

### N-((5,6-dichloro-1H-benzo[d]imidazol-2-yl)methyl)-6-morpholino-3-(1H-pyrazol-4-yl) imidazo[1,2-b]pyridazin-8-amine (61)

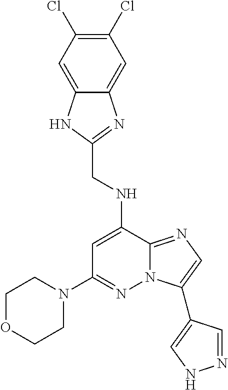 [SMILES: Clc1cc2nc(CNc3cc(N4CCOCC4)nn4c(-c5cn[nH]c5)cnc34)[nH]c2cc1Cl]

Synthesized according to procedure described for 5. Yield (6.50 mg, 11.64%), pale-yellow solid. ¹H NMR (500 MHz, DMSO) δ 8.30 (s, 2H), 7.77 (s, 1H), 7.66 (s, 1H), 7.56 (t, J=6.2 Hz, 1H), 6.01 (s, 1H), 4.82 (d, J=6.2 Hz, 2H), 3.78-3.64 (m, 4H), 3.35 (t, J=4.8 Hz, 4H). LCMS (m/z): 485.1 (M+1), Rt: 2.289 min.

### 6-chloro-8-ethoxy-3-(thiophen-3-yl) imidazo[1,2-b]pyridazine (3f)

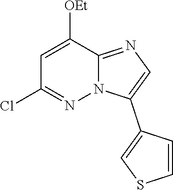 [SMILES: CCOc1cc(Cl)nn2c(-c3ccsc3)cnc12]

Following the general procedure step 3, 3-bromo-6-chloro-8-ethoxyimidazo[1,2-b]pyridazine (1.0 g, 3.62 mmol), 1thiophen-3-ylboronic acid (509 mg, 3.98 mmol), K₂CO₃ (1.0 g, 7.23 mmol), 1,4-dioxane (8 mL) and water (2 mL) for 10 min. To this mixture, Pd(PPh₃)₄ (417 mg) was added, and the reaction was heated to 90° C. for 16 h. Yield (0.80 g, 79.08%), Off-white solid. ¹H NMR (500 MHz, DMSO) δ 8.29 (dd, J=2.9, 1.3 Hz, 1H), 8.14 (s, 1H), 7.79 (dd, J=5.1, 1.3 Hz, 1H), 7.73 (dd, J=5.1, 2.9 Hz, 1H), 6.99 (s, 1H), 4.43 (q, J=7.0 Hz, 2H), 1.46 (t, J=7.0 Hz, 3H). LCMS (m/z): 280.0 (M+1), Rt: 2.969 min.

### 6-chloro-N-((5,6-dichloro-1H-benzo[d]imidazol-2-yl)methyl)-3-(thiophen-3-yl) imidazo[1,2-b]pyridazin-8-amine (62)

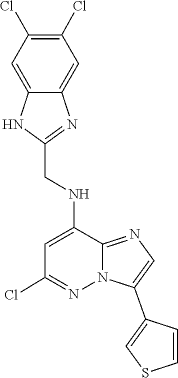 [SMILES: Clc1cc(NCc2nc3cc(Cl)c(Cl)cc3[nH]2)c2ncc(-c3ccsc3)n2n1]

Synthesized according to procedure described for 4. Yield (150 mg, 46.65%). Pale-yellow solid. LCMS (m/z): 451.9 (M+1), Rt: 3.380 min.

### N-((5,6-dichloro-1H-benzo[d]imidazol-2-yl)methyl)-6-(4-methylpiperazin-1-yl)-3-(thiophen-3-yl) imidazo[1,2-b]pyridazin-8-amine (63)

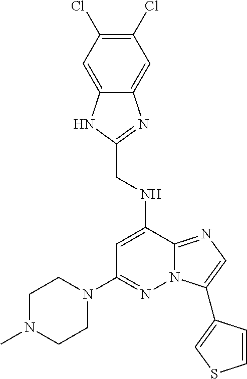 [SMILES: CN1CCN(c2cc(NCc3nc4cc(Cl)c(Cl)cc4[nH]3)c3ncc(-c4ccsc4)n3n2)CC1]

Synthesized according to procedure described for 5. Yield (4 mg, 11.44%), Pale-brown solid. ¹H NMR (500 MHz, DMSO) δ 8.28 (d, J=3.0 Hz, 1H), 8.20 (s, 1H), 7.83 (s, 1H), 7.77-7.71 (m, 2H), 7.65 (dd, J=5.1, 2.9 Hz, 1H), 7.58 (t, J=6.2 Hz, 1H), 6.06 (s, 1H), 4.82 (d, J=6.2 Hz, 2H), 3.40 (t, J=4.5 Hz, 4H), 2.41 (t, J=5.0 Hz, 4H), 2.20 (s, 3H). LCMS (m/z): 514.0 (M+1), Rt: 2.236 min.

### N-((5,6-dichloro-1H-benzo[d]imidazol-2-yl)methyl)-6-morpholino-3-(thiophen-3-yl) imidazo[1,2-b]pyridazin-8-amine (64)

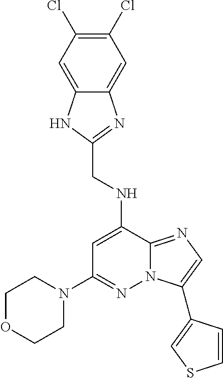 [SMILES: Clc1cc2nc(CNc3cc(N4CCOCC4)nn4c(-c5ccsc5)cnc34)[nH]c2cc1Cl]

Synthesized according to procedure described for 5. Yield (10 mg, 29.85%), Pale-brown solid. ¹H NMR (500 MHz, DMSO) δ 12.62 (s, 1H), 8.29 (d, J=3.0 Hz, 1H), 7.85 (s, 2H), 7.74 (d, J=5.1 Hz, 1H), 7.67-7.61 (m, 2H), 6.07 (s, 1H), 4.83 (d, J=6.2 Hz, 2H), 3.72 (t, J=4.7 Hz, 4H), 3.37 (dd, J=6.0, 4.0 Hz, 4H). LCMS (m/z): 501.1 (M+1), Rt: 2.949 min.

### 6-chloro-N-((7-chloro-1H-benzo[d]imidazol-2-yl)methyl)-3-(thiophen-3-yl) imidazo[1,2-b]pyridazin-8-amine (65)

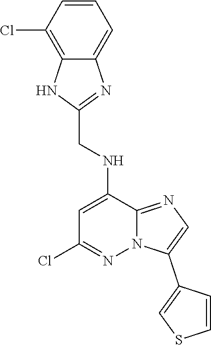 [SMILES: Clc1cc(NCc2nc3cccc(Cl)c3[nH]2)c2ncc(-c3ccsc3)n2n1]

Synthesized according to procedure described for 4. Yield (200 mg, 67.36%). Pale-yellow solid. ¹H NMR (500 MHz, DMSO) δ 12.76 (s, 1H), 8.44 (d, J=18.9 Hz, 1H), 8.27 (dd, J=3.0, 1.3 Hz, 1H), 8.06 (s, 1H), 7.77 (dd, J=5.1, 1.3 Hz, 1H), 7.71 (dd, J=5.1, 2.9 Hz, 1H), 7.50-7.40 (m, 1H), 7.24 (d, J=7.6 Hz, 1H), 7.16 (t, J=7.9 Hz, 1H), 6.40 (s, 1H), 4.98-4.78 (m, 2H). LCMS (m/z): 416.0 (M+1), Rt: 3.110 min.

### N-((7-chloro-1H-benzo[d]imidazol-2-yl)methyl)-6-(4-methylpiperazin-1-yl)-3-(thiophen-3-yl) imidazo[1,2-b]pyridazin-8-amine (66)

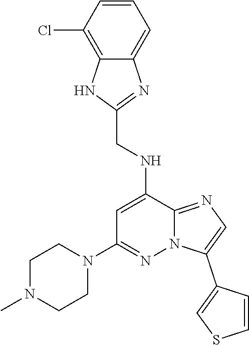 [SMILES: CN1CCN(c2cc(NCc3nc4cccc(Cl)c4[nH]3)c3ncc(-c4ccsc4)n3n2)CC1]

Synthesized according to procedure described for 5. Yield (15 mg, 26.01%), Pale-yellow solid. ¹H NMR (500 MHz, DMSO) δ 12.68 (s, 1H), 8.28 (dd, J=3.1, 1.2 Hz, 1H), 8.16 (s, 1H), 7.83 (s, 1H), 7.74 (dd, J=5.1, 1.2 Hz, 1H), 7.65 (dd, J=5.1, 2.9 Hz, 1H), 7.44 (d, J=8.0 Hz, 1H), 7.23 (d, J=7.7 Hz, 1H), 7.15 (t, J=7.9 Hz, 1H), 6.15 (s, 1H), 4.83 (d, J=6.0 Hz, 2H), 3.41 (t, J=4.9 Hz, 4H), 2.43 (t, J=4.9 Hz, 4H), 2.21 (s, 3H). LCMS (m/z): 480.1 (M+1), Rt: 2.012 min.

### N-((7-chloro-1H-benzo[d]imidazol-2-yl)methyl)-6-morpholino-3-(thiophen-3-yl) imidazo[1,2-b]pyridazin-8-amine (67)

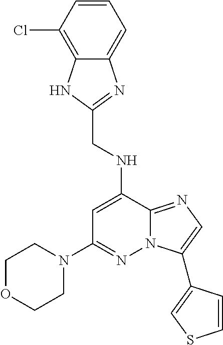 [SMILES: Clc1cccc2nc(CNc3cc(N4CCOCC4)nn4c(-c5ccsc5)cnc34)[nH]c12]

Synthesized according to procedure described for 5. Yield (9 mg, 16.04%), Pale-yellow solid. ¹H NMR (500 MHz, DMSO) δ 12.66 (s, 1H), 8.29 (d, J=3.0 Hz, 1H), 7.84 (s, 1H), 7.74 (d, J=5.1 Hz, 1H), 7.69-7.61 (m, 2H), 7.41 (d, J=8.0 Hz, 1H), 7.22 (d, J=7.8 Hz, 1H), 7.15 (t, J=7.8 Hz, 1H), 6.15 (d, J=18.5 Hz, 1H), 4.84 (d, J=6.2 Hz, 2H), 3.72 (t, J=4.8 Hz, 4H), 3.38 (1, J=4.7 Hz, 4H). LCMS (m/z): 466.1 (M+1), Rt: 2.665 min.

### N-((7-chloro-1H-benzo[d]imidazol-2-yl)methyl)-6-(piperazin-1-yl)-3-(thiophen-3-yl) imidazo[1,2-b]pyridazin-8-amine (68)

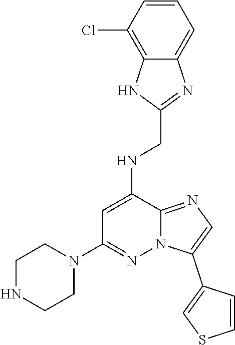 [SMILES: Clc1cccc2nc(CNc3cc(N4CCNCC4)nn4c(-c5ccsc5)cnc34)[nH]c12]

Synthesized according to procedure described for 5. Yield (9 mg, 28.34%), Pale-yellow solid. ¹H NMR (500 MHz, DMSO) δ 12.70 (s, 1H), 8.28 (dd, J=3.0, 1.2 Hz, 1H), 8.21 (s, 1H), 7.84 (s, 1H), 7.74 (dd, J=5.1, 1.2 Hz, 1H), 7.65 (dd, J=5.1, 3.0 Hz, 2H), 7.44 (d, J=7.8 Hz, 1H), 7.23 (d, J=7.7 Hz, 1H), 7.15 (t. J=7.8 Hz, 1H), 6.15 (s, 1H), 4.83 (d, J=6.1 Hz, 2H), 3.46-3.40 (m, 4H), 2.97-2.86 (m, 4H). LCMS (m/z): 465.1 (M+1), Rt: 1.990 min.

### 6-(3-aminopyrrolidin-1-yl)-N-((7-chloro-1H-benzo[d]imidazol-2-yl)methyl)-3-(thiophen-3-yl) imidazo[1,2-b]pyridazin-8-amine (69)

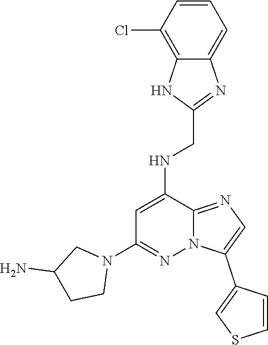 [SMILES: NC1CCN(c2cc(NCc3nc4cccc(Cl)c4[nH]3)c3ncc(-c4ccsc4)n3n2)C1]

Synthesized according to procedure described for 5. Yield (10 mg, 30.38%), Off-white solid. ¹H NMR (500 MHz, DMSO) δ 8.36 (dd, J=2.9, 1.3 Hz, 1H), 8.26 (s, 1H), 7.81 (s, 1H), 7.78-7.75 (m, 1H), 7.65 (dd, J=5.1, 3.0 Hz, 1H), 7.60 (s, 1H), 7.45 (d, J=8.0 Hz, 1H), 7.23 (dd, J=7.8, 1.0 Hz, 1H), 7.15 (t, J=7.9 Hz, 1H), 5.75 (d, J=5.2 Hz, 1H), 4.83 (d, J=5.9 Hz, 2H), 3.77 (p, J=5.4 Hz, 1H), 3.62 (dd, J=10.9, 6.0 Hz, 1H), 3.57-3.52 (m, 1H), 3.47-3.38 (m, 2H), 2.23-2.16 (m, 1H), 1.91 (t, J=7.6, 3.9 Hz, 1H). LCMS (m/z): 465.1 (M+1), Rt: 1.996 min.

### 6-(4-aminopiperidin-1-yl)-N-((7-chloro-1H-benzo[d]imidazol-2-yl)methyl)-3-(thiophen-3-yl) imidazo[1,2-b]pyridazin-8-amine (70)

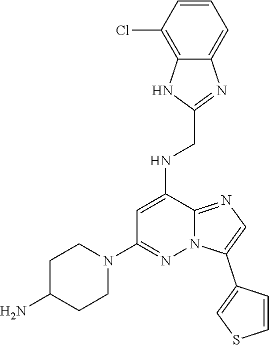 [SMILES: NC1CCN(c2cc(NCc3nc4cccc(Cl)c4[nH]3)c3ncc(-c4ccsc4)n3n2)CC1]

Synthesized according to procedure described for 5. Yield (8 mg, 24.18%), Off-white solid. ¹H NMR (500 MHz, DMSO) δ 8.39 (s, 1H), 8.27 (dd, J=3.0, 1.3 Hz, 1H), 7.83 (s, 1H), 7.74 (dd, J=5.1, 1.2 Hz, 1H), 7.68-7.57 (m, 2H), 7.44 (d, J=7.9 Hz, 1H), 7.22 (dd, J=7.8, 0.9 Hz, 1H), 7.15 (t, J=7.9 Hz, 1H), 6.16 (s, 1H), 4.83 (d, J=6.0 Hz, 2H), 4.08 (dd, J=13.3, 3.8 Hz, 2H), 3.11 (dd, J=15.6, 6.4 Hz, 1H), 2.92 (dd, J=25.5, 2.5 Hz, 2H), 1.93-1.81 (m, 2H), 1.45 (dd, J=12.1, 4.0 Hz, 2H). LCMS (m/z): 480.1 (M+1), Rt: 2.025 min.

### N8-((7-chloro-1H-benzo[d]imidazol-2-yl)methyl)-N6-(1-methylpiperidin-4-yl)-3-(thiophen-3-yl) imidazo[1,2-b]pyridazine-6,8-diamine (71)

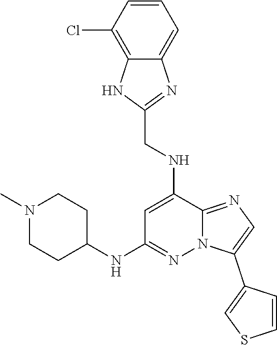 [SMILES: CN1CCC(Nc2cc(NCc3nc4cccc(Cl)c4[nH]3)c3ncc(-c4ccsc4)n3n2)CC1]

Synthesized according to procedure described for 5. Yield (7 mg, 12.79%), Off-white solid. ¹H NMR (500 MHz, DMSO) δ 12.68 (s, 1H), 8.28 (dd, J=3.1, 1.2 Hz, 1H), 8.16 (s, 1H), 7.83 (s, 1H), 7.74 (dd, J=5.1, 1.2 Hz, 1H), 7.65 (dd, J=5.1, 2.9 Hz, 1H), 7.59 (s, 1H), 7.44 (d, J=8.0 Hz, 1H), 7.23 (d, J=7.7 Hz, 1H), 7.15 (t, J=7.9 Hz, 1H), 6.15 (s, 1H), 4.83 (d, J=6.0 Hz, 2H), 3.41 (t, J=4.9 Hz, 4H), 2.43 (t, J=4.9 Hz, 4H), 2.21 (s, 3H). LCMS (m/z): 491.2 (M-2), Rt: 2.003 min.

### N8-((7-chloro-1H-benzo[d]imidazol-2-yl)methyl)-N6-(piperidin-4-yl)-3-(thiophen-3-yl) imidazo[1,2-b]pyridazine-6,8-diamine (72)

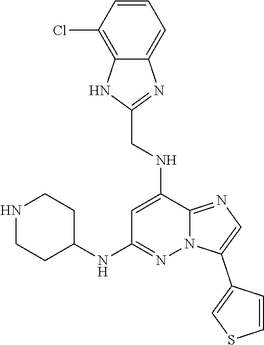 [SMILES: Clc1cccc2nc(CNc3cc(NC4CCNCC4)nn4c(-c5ccsc5)cnc34)[nH]c12]

Synthesized according to procedure described for 5. Yield (15 mg, 26.01%), Off-white solid. ¹H NMR (500 MHz, DMSO) δ 8.29 (s, 1H), 8.26 (dd, J=3.0, 1.2 Hz, 1H), 7.79 (s, 1H), 7.73 (dd, J=5.1, 1.2 Hz, 1H), 7.64 (dd, J=5.1, 3.0 Hz, 1H), 7.57 (q, J=8.8, 6.0 Hz, 1H), 7.44 (d, J=7.9 Hz, 1H), 7.22 (dd, J=7.8, 0.9 Hz, 1H), 7.14 (t, J=7.9 Hz, 1H), 5.93 (s, 1H), 4.82 (d, J=6.0 Hz, 2H), 3.67 (t, J=5.2 Hz, 2H), 3.63 (t, J=6.2 Hz, 2H), 2.94 (t, J=5.2 Hz, 2H), 2.70 (dd, J=7.0, 4.6 Hz, 1H), 1.79 (t, J=6.0 Hz, 2H). LCMS (m/z): 477.1 (M−2), Rt: 2.074 min.

### N8-((7-chloro-1H-benzo[d]imidazol-2-yl)methyl)-N6,N6-dimethyl-3-(thiophen-3-yl) imidazo[1,2-b]pyridazine-6,8-diamine (73)

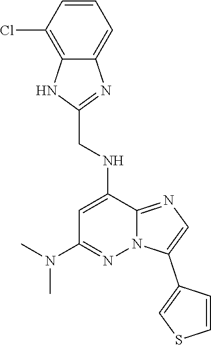 [SMILES: CN(C)c1cc(NCc2nc3cccc(Cl)c3[nH]2)c2ncc(-c3ccsc3)n2n1]

Synthesized according to procedure described for 5. Yield (8 mg, 15.67%), Off-white solid. ¹H NMR (500 MHz, DMSO) δ 12.68 (s, 1H), 8.33 (dd, J=3.0, 1.2 Hz, 1H), 7.80 (s, 1H), 7.75 (dd, J=5.1, 1.3 Hz, 1H), 7.64 (dd, J=5.1, 3.0 Hz, 1H), 7.58 (t, J=6.0 Hz, 1H), 7.41 (d, J=7.9 Hz, 1H), 7.22 (d, J=7.7 Hz, 1H), 7.15 (t, J=7.8 Hz, 1H), 5.94 (s, 1H), 4.83 (d, J=5.9 Hz, 2H), 3.00 (d, J=6.4 Hz, 6H). LCMS (m/z): 424.1 (M+2), Rt: 2.703 min.

### 6-chloro-N-((7-methyl-3H-imidazo[4,5-b]pyridin-2-yl)methyl)-3-(thiophen-3-yl) imidazo[1,2-b]pyridazin-8-amine (74)

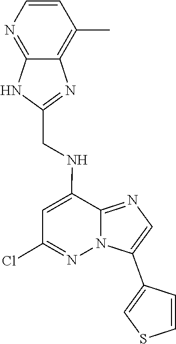 [SMILES: Cc1ccnc2[nH]c(CNc3cc(Cl)nn4c(-c5ccsc5)cnc34)nc12]

Synthesized according to procedure described for 4. Yield (200 mg, 70.67%). Pale-yellow solid. LCMS (m/z): 396.0 (M+1), Rt: 2.442 min.

### N-((7-methyl-3H-imidazo[4,5-b]pyridin-2-yl)methyl)-6-(4-methylpiperazin-1-yl)-3-(thiophen-3-yl) imidazo[1,2-b]pyridazin-8-amine (75)

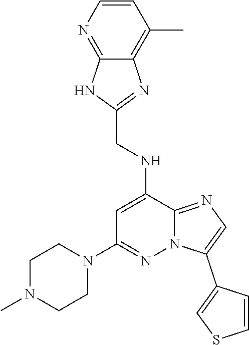 [SMILES: Cc1ccnc2[nH]c(CNc3cc(N4CCN(C)CC4)nn4c(-c5ccsc5)cnc34)nc12]

Synthesized according to procedure described for 5. Yield (15 mg, 25.84%), Pale-yellow solid. ¹H NMR (500 MHz, DMSO) δ 8.28 (dd, J=3.1, 1.2 Hz, 1H), 8.19-8.12 (m, 2H), 7.83 (s, 1H), 7.74 (dd, J=5.0, 1.2 Hz, 1H), 7.65 (dd, J=5.0, 2.9 Hz, 1H), 7.50 (t, J=6.0 Hz, 1H), 7.02 (d, J=4.9 Hz, 1H), 6.13 (s, 1H), 4.81 (d, J=5.9 Hz, 2H), 3.43-3.39 (m, 4H), 2.52 (s, 3H), 2.43 (t, J=5.0 Hz, 4H), 2.21 (s, 3H). LCMS (m/z): 460.1 (M+1), Rt: 1.688 min.

### N-((7-methyl-3H-imidazo[4,5-b]pyridin-2-yl)methyl)-6-morpholino-3-(thiophen-3-yl) imidazo[1,2-b]pyridazin-8-amine (76)

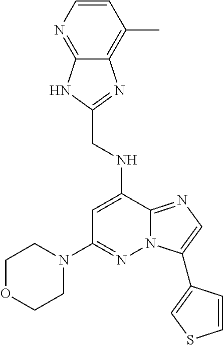 [SMILES: Cc1ccnc2[nH]c(CNc3cc(N4CCOCC4)nn4c(-c5ccsc5)cnc34)nc12]

Synthesized according to procedure described for 5. Yield (16 mg, 28.37%), Pale-yellow solid. ¹H NMR (500 MHz, DMSO) δ 12.79 (s, 1H), 8.29 (d, J=3.0 Hz, 1H), 8.14 (s, 1H), 7.84 (s, 1H), 7.74 (d, J=5.1 Hz, 1H), 7.65 (dd, J=5.1, 2.9 Hz, 1H), 7.54 (t, J=6.0 Hz, 1H), 7.02 (d, J=4.9 Hz, 1H), 6.14 (s, 1H), 4.81 (d, J=5.9 Hz, 2H), 3.73 (t, J=4.8 Hz, 4H), 3.39 (t, J=4.9 Hz, 4H), 2.52 (s, 3H). LCMS (m/z): 447.1 (M+1), Rt: 2.171 min.

### 6-chloro-N-((4,5-difluoro-1H-benzo[d]imidazol-2-yl)methyl)-3-(thiophen-3-yl)imidazo[1,2-b]pyridazin-8-amine (77)

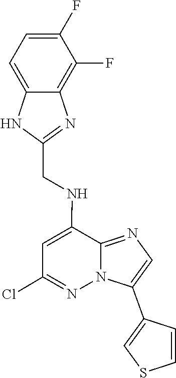 [SMILES: Fc1ccc2[nH]c(CNc3cc(Cl)nn4c(-c5ccsc5)cnc34)nc2c1F]

Synthesized according to procedure described for 4. Yield (100 mg, 33.56%). Pale-yellow solid. LCMS (m/z): 417.0 (M+1), Rt: 3.112 min.

### N-((4,5-difluoro-1H-benzo[d]imidazol-2-yl)methyl)-6-(4-methylpiperazin-1-yl)-3-(thiophen-3-yl) imidazo[1,2-b]pyridazin-8-amine (78)

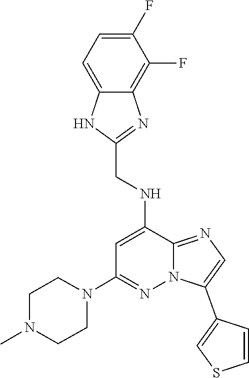 [SMILES: CN1CCN(c2cc(NCc3nc4c(F)c(F)ccc4[nH]3)c3ncc(-c4ccsc4)n3n2)CC1]

Synthesized according to procedure described for 5. Yield (4 mg, 6.94%), Pale-brown solid. ¹H NMR (500 MHz, DMSO) δ 8.28 (d, J=3.0 Hz, 1H), 8.23 (s, 1H), 7.83 (s, 1H), 7.74 (d, J=5.1 Hz, 1H), 7.65 (dd, J=5.2, 3.0 Hz, 1H), 7.59 (s, 1H), 7.26 (s, 1H), 7.23-7.18 (m, 1H), 6.09 (s, 1H), 4.83 (d, J=6.1 Hz, 2H), 3.40 (t, J=5.1 Hz, 4H), 2.41 (t, J=5.0 Hz, 4H), 2.20 (s, 3H). LCMS (m/z): 481.1 (M+1), Rt: 2.057 min.

### 4-(6-chloro-8-ethoxyimidazo[1,2-b]pyridazin-3-yl)thiophene-2-carbonitrile (3g)

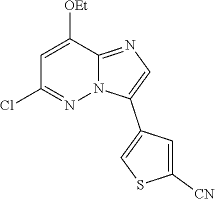 [SMILES: CCOc1cc(Cl)nn2c(-c3csc(C#N)c3)cnc12]

Following the general procedure step 3, 3-bromo-6-chloro-8-ethoxyimidazo[1,2-b]pyridazine (1.0 g, 3.62 mmol), (5-cyanothiophen-3-yl) boronic acid (609 mg, 3.98 mmol), K₂CO₃ (1.0 g, 7.23 mmol), 1,4-dioxane (8 mL) and water (2 mL) for 10 min. To this mixture, Pd(PPh₃)₄ (417 mg) was added, and the reaction was heated to 90° C. for 16 h. Yield (0.50 g, 45.37%), Pale-yellow solid. LCMS (m/z): 305.0 (M+1), Rt: 2.917 min.

### 4-(8-(((5,6-dichloro-1H-benzo[d]imidazol-2-yl)methyl)amino)-6-(4-methylpiperazin-1-yl) imidazo[1,2-b]pyridazin-3-yl)thiophene-2-carbonitrile (79)

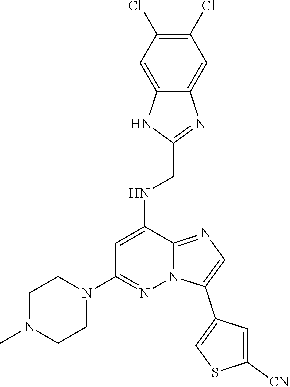 [SMILES: CN1CCN(c2cc(NCc3nc4cc(Cl)c(Cl)cc4[nH]3)c3ncc(-c4csc(C#N)c4)n3n2)CC1]

Synthesized according to procedure described for 5. Yield (3 mg, 5.49%), Pale-brown solid. ¹H NMR (500 MHz, DMSO) δ 8.72 (d, J=1.5 Hz, 1H), 8.57 (d, J=1.5 Hz, 1H), 8.19 (s, 1H), 7.93 (s, 1H), 7.65 (t, J=6.2 Hz, 2H), 6.09 (s, 1H), 4.83 (d, J=6.3 Hz, 2H), 3.41 (s, 4H), 2.40 (q, J=5.5 Hz, 4H), 2.22-2.18 (m, 3H). LCMS (m/z): 539.1 (M+1), Rt: 2.327 min.

### 4-(8-(((5,6-dichloro-1H-benzo[d]imidazol-2-yl)methyl)amino)-6-morpholinoimidazo[1,2-b]pyridazin-3-yl)thiophene-2-carbonitrile (80)

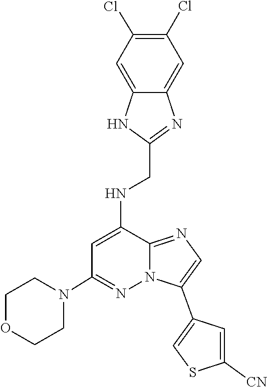 [SMILES: N#Cc1cc(-c2cnc3c(NCc4nc5cc(Cl)c(Cl)cc5[nH]4)cc(N4CCOCC4)nn23)cs1]

Synthesized according to procedure described for 5. Yield (3 mg, 5.62%), Pale-brown solid. ¹H NMR (500 MHz, DMSO) δ 8.73 (d, J=1.5 Hz, 1H), 8.57 (d, J=1.5 Hz, 1H), 8.43-8.37 (m, 1H), 7.95 (s, 1H), 7.78 (d, J=8.8 Hz, 2H), 7.70 (t, J=6.2 Hz, 1H), 6.11 (s, 1H), 4.83 (d, J=6.2 Hz, 2H), 3.76-3.68 (m, 4H), 3.38 (t, J=4.8 Hz, 4H). LCMS (m/z): 526.0 (M+1), Rt: 3.068 min.

### 4-(8-(((7-chloro-1/-benzo[d]imidazol-2-yl)methyl)amino)-6-(4-methylpiperazin-1-yl) imidazo[1,2-b]pyridazin-3-yl)thiophene-2-carbonitrile (81)

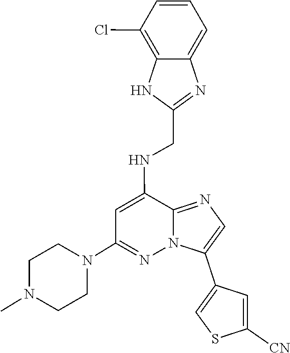 [SMILES: CN1CCN(c2cc(NCc3nc4cccc(Cl)c4[nH]3)c3ncc(-c4csc(C#N)c4)n3n2)CC1]

Synthesized according to procedure described for 5. Yield (3 mg, 5.46%), Pale-brown solid. ¹H NMR (500 MHz, DMSO) δ 8.72 (d, J=1.4 Hz, 1H), 8.57 (d, J=1.5 Hz, 1H), 7.93 (s, 1H), 7.44 (d, J=8.0 Hz, 1H), 7.23 (d, J=7.6 Hz, 1H), 7.15 (t, J=7.9 Hz, 1H), 6.19 (s, 1H), 4.83 (d, J=6.0 Hz, 2H), 2.99 (s, 3H), 2.82 (dd, J=8.4, 3.6 Hz, 4H), 2.15 (d, J=4.0 Hz, 4H). LCMS (m/z): 505.0 (M+1), Rt: 2.103 min.

### 4-(8-(((4,5-difluoro-1H-benzo[d]imidazol-2-yl)methyl)amino)-6-(4-methylpiperazin-1-yl) imidazo[1,2-b]pyridazin-3-yl)thiophene-2-carbonitrile (82)

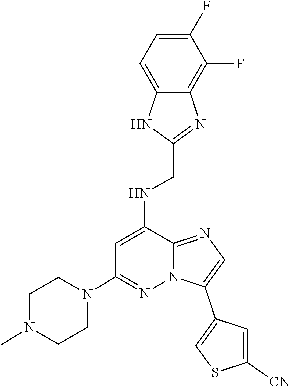 [SMILES: CN1CCN(c2cc(NCc3nc4c(F)c(F)ccc4[nH]3)c3ncc(-c4csc(C#N)c4)n3n2)CC1]

Synthesized according to procedure described for 5. Yield (3.40 mg, 6.18%), Pale-brown solid. ¹H NMR (500 MHz, DMSO) δ 8.72 (s, 1H), 8.57 (s, 1H), 8.21 (s, 1H), 7.93 (s, 1H), 7.66 (t, J=5.9 Hz, 1H), 7.30-7.18 (m, 2H), 6.13 (s, 1H), 4.83 (d, J=6.2 Hz, 2H), 3.41 (d, J=10.0 Hz, 4H), 2.40 (q, J=7.5, 6.2 Hz, 4H), 2.19 (d, J=12.4 Hz, 3H). ¹⁹F NMR (471 MHz, DMSO) δ −150.08, −154.88 (s). LCMS (m/z): 506.1 (M+1), Rt: 2.117 min.

### 6-chloro-8-ethoxy-3-(5-methylthiophen-3-yl) imidazo[1,2-b]pyridazine (3h)

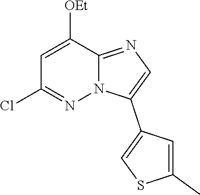 [SMILES: CCOc1cc(Cl)nn2c(-c3csc(C)c3)cnc12]

Following the general procedure step 3, 3-bromo-6-chloro-8-ethoxyimidazo[1,2-b]pyridazine (1.50 g, 5.42 mmol), (5-methyl thiophen-3-yl) boronic acid (808 mg, 5.70 mmol), K₂CO₃ (1.87 g, 13.56 mmol), 1,4-dioxane (16 mL) and water (4 mL) for 10 min. To this mixture, Pd(PPh₃)₄ (627 mg) was added, and the reaction was heated to 90° C. for 16 h. Yield (1.0 g, 62.75%), Pale-brown gummy solid. ¹H NMR (500 MHz, DMSO) δ 8.06 (s, 1H), 8.03 (d, J=1.4 Hz, 1H), 7.46 (t, J=1.3 Hz, 1H), 6.98 (s, 1H), 4.42 (q, J=7.0 Hz, 2H), 2.51 (d, J=1.1 Hz, 3H), 1.45 (t, J=7.0 Hz, 3H). LCMS (m/z): 294.0 (M+1), Rt: 3.115 min.

### 6-chloro-N-((7-chloro-1H-benzo[d]imidazol-2-yl)methyl)-3-(5-methylthiophen-3-yl)imidazo[1,2-b]pyridazin-8-amine (83)

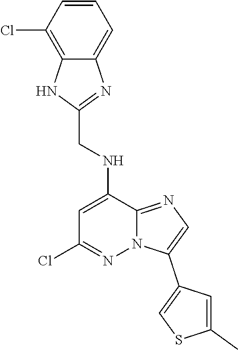 [SMILES: Cc1cc(-c2cnc3c(NCc4nc5cccc(Cl)c5[nH]4)cc(Cl)nn23)cs1]

Synthesized according to procedure described for 4. Yield (140 mg, 38.32%). Pale-brown solid. ¹H NMR (500 MHz, DMSO) δ 12.72 (s, 1H), 8.48 (s, 1H), 8.04-7.95 (m, 2H), 7.45 (t, J=1.4 Hz, 1H), 7.24 (d, J=7.8 Hz, 1H), 7.16 (t, J=7.8 Hz, 1H), 6.38 (s, 1H), 4.88 (s, 2H), 2.48 (s, 3H). LCMS (m/z): 430.0 (M+1), Rt: 3.210 min.

### N-((7-chloro-1H-benzo[d]imidazol-2-yl)methyl)-6-(4-methylpipcrazin-1-yl)-3-(5-methylthiophen-3-yl) imidazo[1,2-b]pyridazin-8-amine (84)

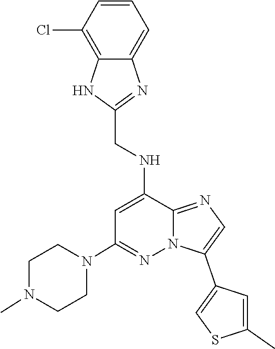

Synthesized according to procedure described for 5. Yield (17 mg, 29.61%), Off-white solid. ¹H NMR (500 MHz, DMSO) δ 12.69 (s, 1H), 8.17 (s, 1H), 8.03 (d. J=1.4 Hz, 1H), 7.75 (s, 1H), 7.43 (s, 1H), 7.40 (t, J=1.3 Hz, 1H), 7.23 (dd, J=7.8, 0.9 Hz, 1H), 7.15 (t, J=7.8 Hz, 1H), 6.14 (s, 1H), 4.82 (d, J=6.1 Hz, 2H), 3.41-3.38 (m, 4H), 2.49 (d, J=1.1 Hz, 3H), 2.41 (L, J=5.0 Hz, 4H), 2.20 (s, 3H). LCMS (m/z): 494.1 (M+1), Rt: 2.069 min.

### N-((7-chloro-1H-benzo[d]imidazol-2-yl)methyl)-3-(5-methylthiophen-3-yl)-6-morpholinoimidazo[1,2-b]pyridazin-8-amine (85)

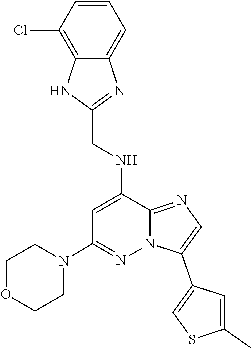 [SMILES: Cc1cc(-c2cnc3c(NCc4nc5cccc(Cl)c5[nH]4)cc(N4CCOCC4)nn23)cs1]

Synthesized according to procedure described for 5. Yield (9 mg, 16.10%), Off-white solid. ¹H NMR (500 MHz, DMSO) δ 12.68 (s, 1H), 8.05 (d, J=1.3 Hz, 1H). 7.77 (s, 1H), 7.65 (s, 1H), 7.46-7.35 (m, 2H), 7.23 (d, J=7.7 Hz, 1H), 7.15 (t, J=7.8 Hz, 1H), 6.15 (s, 1H), 4.83 (d, J=6.1 Hz, 2H), 3.72 (t, J=4.8 Hz, 4H), 3.37 (t, J=4.8 Hz, 4H), 2.49 (d, J=1.1 Hz, 3H). LCMS (m/z): 480.1 (M+1), Rt: 2.762 min.

### 6-chloro-N-((5,6-dichloro-1H-benzo[d]imidazol-2-yl)methyl)-3-(5-methylthiophen-3-yl) imidazo[1,2-b]pyridazin-8-amine (86)

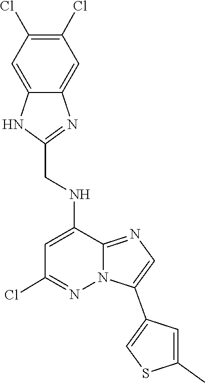 [SMILES: Cc1cc(-c2cnc3c(NCc4nc5cc(Cl)c(Cl)cc5[nH]4)cc(Cl)nn23)cs1]

Synthesized according to procedure described for 4. Yield (150 mg, 38.01%). Pale-brown solid. ¹H NMR (500 MHz, DMSO) δ 12.69 (s, 1H), 8.44 (t, J=6.3 Hz, 1H), 8.04-7.96 (m, 2H), 7.45 (t, J=1.3 Hz, 1H), 6.37 (s, 1H), 4.86 (s, 2H), 4.11 (q, J=5.3 Hz, 1H), 3.16 (d, J=4.7 Hz, 2H). LCMS (m/z): 465.0 (M+1), Rt: 3.502 min.

### N-((5,6-dichloro-1H-benzo[d]imidazol-2-yl)methyl)-6-(4-methylpiperazin-1-yl)-3-(5-methylthiophen-3-yl) imidazo[1,2-b]pyridazin-8-amine (87)

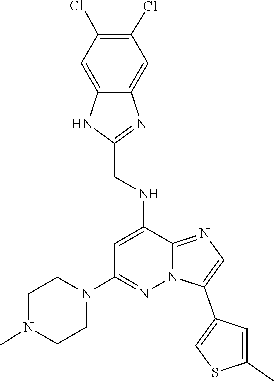

Synthesized according to procedure described for 5. Yield (18 mg, 31.65%), Off-white solid. ¹H NMR (500 MHz, DMSO) δ 8.16 (s, 1H), 8.04 (d, J=1.4 Hz, 1H), 7.76 (s, 2H), 7.59 (t, J=6.2 Hz, 1H), 7.40 (t, J=1.3 Hz, 1H), 6.05 (s, 1H), 4.82 (d, J=6.2 Hz, 2H), 3.39 (dd, J=6.4, 3.8 Hz, 4H), 2.49 (s, 3H), 2.42 (t, J=5.0 Hz, 4H), 2.21 (s, 3H). LCMS (m/z): 528.1 (M+1), Rt: 2.264 min.

### N-((5,6-dichloro-1H-benzo[d]imidazol-2-yl)methyl)-3-(5-methylthiophen-3-yl)-6-morpholinoimidazo[1,2-b]pyridazin-8-amine (88)

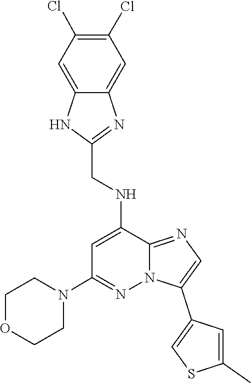 [SMILES: Cc1cc(-c2cnc3c(NCc4nc5cc(Cl)c(Cl)cc5[nH]4)cc(N4CCOCC4)nn23)cs1]

Synthesized according to procedure described for 5. Yield (17 mg, 30.65%), Pale-yellow solid. ¹H NMR (500 MHz, DMSO) δ 12.63 (s, 1H), 8.05 (s, 1H), 7.77 (s, 2H), 7.62 (t, J=6.2 Hz, 1H), 7.40 (s, 1H), 6.06 (s, 1H), 4.82 (d, J=6.1 Hz, 2H), 3.71 (t, J=4.7 Hz, 4H), 3.36 (t, J=4.9 Hz, 4H), 2.49 (s, 3H). LCMS (m/z): 515.1 (M+1), Rt: 3.024 min.

### 6-chloro-N-((7-methyl-3H-imidazo[4,5-b]pyridin-2-yl)methyl)-3-(5-methylthiophen-3-yl)imidazo[1,2-b]pyridazin-8-amine (89)

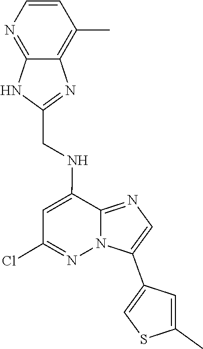 [SMILES: Cc1cc(-c2cnc3c(NCc4nc5c(C)ccnc5[nH]4)cc(Cl)nn23)cs1]

Synthesized according to procedure described for 4. Yield (140 mg, 59.02%). Pale-brown solid. ¹H NMR (500 MHz, DMSO) δ 12.88 (s, 1H), 8.40 (t, J=5.9 Hz, 1H), 8.14 (d, J=4.9 Hz, 1H), 8.05-7.95 (m, 2H), 7.44 (t, J=1.4 Hz, 1H), 7.02 (d, J=4.9 Hz, 1H), 6.39 (s, 1H), 4.85 (s, 2H), 2.51 (s, 3H), 2.49 (s, 3H). LCMS (m/z): 410.1 (M+1), Rt: 2.564 min.

### N-((7-methyl-3H-imidazo[4,5-b]pyridin-2-yl)methyl)-6-(4-methylpiperazin-1-yl)-3-(5-methylthiophen-3-yl) imidazo[1,2-b]pyridazin-8-amine (90)

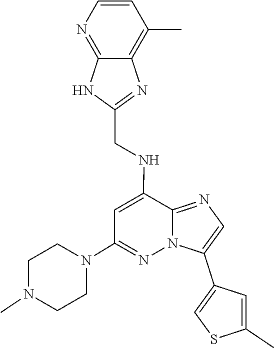

Synthesized according to procedure described for 5. Yield (20 mg, 34.62%), Off-white solid. ¹H NMR (500 MHz, DMSO) δ 8.17 (s, 2H), 8.03 (d, J=1.3 Hz, 1H), 7.75 (s, 1H), 7.50 (t, J=6.0 Hz, 1H), 7.40 (d, J=1.4 Hz, 1H), 7.02 (d, J=4.9 Hz, 1H), 6.11 (s, 1H), 4.80 (d, J=5.9 Hz, 2H), 3.41-3.37 (m, 4H), 2.52 (s, 3H), 2.49 (d, J=1.1 Hz, 3H), 2.42 (t, J=5.0 Hz, 4H), 2.20 (s, 3H). LCMS (m/z): 474.2 (M+1), Rt: 1.773 min.

### N-((7-methyl-3H-imidazo[4,5-b]pyridin-2-yl)methyl)-3-(5-methylthiophen-3-yl)-6-morpholinoimidazo[1,2-b]pyridazin-8-amine (91)

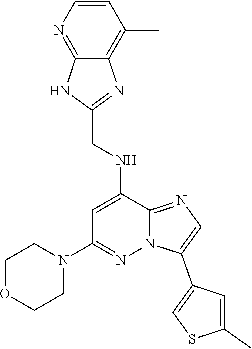 [SMILES: Cc1cc(-c2cnc3c(NCc4nc5c(C)ccnc5[nH]4)cc(N4CCOCC4)nn23)cs1]

Synthesized according to procedure described for 5. Yield (5 mg, 8.90%), Off-white solid. ¹H NMR (500 MHz, DMSO) δ 12.96 (s, 1H), 8.13 (s, 1H), 8.05 (d, J=1.4 Hz., 1H), 7.77 (s, 1H), 7.54 (t, J=6.0 Hz, 1H), 7.40 (t, J=1.4 Hz, 1H), 7.02 (d, J=4.9 Hz, 1H), 6.12 (s, 1H), 4.81 (d, J=6.1 Hz, 2H), 3.72 (t, J=4.8 Hz, 4H), 3.37 (t, J=4.9 Hz, 4H), 2.52 (s, 3H), 2.49 (d, J=1.1 Hz, 3H). LCMS (m/z): 461.1 (M+1), Rt: 2.269 min.

### 6-chloro-N-((4-fluoro-1H-benzo[d]imidazol-2-yl)methyl)-3-(5-methylthiophen-3-yl)imidazo[1,2-b]pyridazin-8-amine (92)

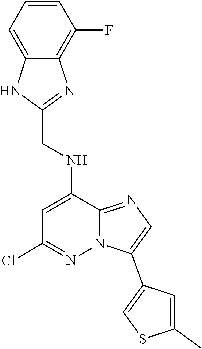 [SMILES: Cc1cc(-c2cnc3c(NCc4nc5c(F)cccc5[nH]4)cc(Cl)nn23)cs1]

Synthesized according to procedure described for 4. Yield (130 mg, 54.41%). Pale-brown solid. ¹H NMR (500 MHz, DMSO) δ 12.68 (s, 1H), 8.45 (s, 1H), 8.01 (d, J=1.5 Hz, 1H), 7.98 (s, 1H), 7.47-7.42 (m, 2H), 6.98 (s, 1H), 6.37 (s, 1H), 4.86 (s, 2H), 2.48 (s, 3H). LCMS (m/z): 413.0 (M+1), Rt: 3.097 min.

### N-((4-fluoro-1H-benzo[d]imidazol-2-yl)methyl)-6-(4-methylpiperazin-1-yl)-3-(5-methylthiophen-3-yl) imidazo[1,2-b]pyridazin-8-amine (93)

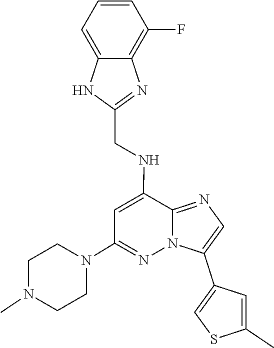

Synthesized according to procedure described for 5. Yield (4 mg, 6.93%), Off-white solid. ¹H NMR (500 MHz, DMSO) δ 12.71 (s, 1H), 8.04 (d, J=1.4 Hz, 1H), 7.76 (s, 1H), 7.58 (d, J=5.6 Hz, 1H), 7.40 (t, J=1.4 Hz, 1H), 7.29 (d. J=8.0 Hz, 1H), 7.12 (td, J=8.0, 4.8 Hz, 1H), 6.96 (dd, J=11.0, 7.9 Hz, 1H), 6.09 (s, 1H), 4.82 (d, J=6.0 Hz, 2H), 3.39 (dd, J=5.9, 4.0 Hz, 4H), 2.49 (d, J=1.1 Hz, 3H), 2.40 (t, J=5.0 Hz, 4H), 2.19 (s, 3H). ¹⁹F NMR (471 MHz, DMSO) δ −129.52. LCMS (m/z): 477.1 (M+1), Rt: 2.078 min.

### N-((4-fluoro-1H-benzo[d]imidazol-2-yl)methyl)-3-(5-methylthiophen-3-yl)-6-morpholinoimidazo[1,2-b]pyridazin-8-amine (94)

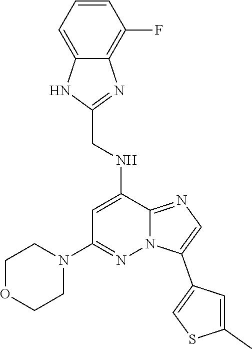 [SMILES: Cc1cc(-c2cnc3c(NCc4nc5c(F)cccc5[nH]4)cc(N4CCOCC4)nn23)cs1]

Synthesized according to procedure described for 5. Yield (5 mg, 8.91%), Off-white solid. ¹H NMR (500 MHz, DMSO) δ 12.63 (d, J=2.3 Hz, 1H), 8.05 (d, J=1.4 Hz, 1H), 7.77 (d, J=2.9 Hz, 1H), 7.63 (t, J=6.2 Hz, 1H), 7.40 (t, J=1.4 Hz, 1H), 7.27 (d, J=8.0 Hz, 1H), 7.13 (td, J=8.0, 4.8 Hz, 1H), 6.95 (dd, J=11.1, 7.9 Hz, 1H), 6.09 (s, 1H), 4.82 (t, J=5.3 Hz, 2H), 3.71 (dd, J=5.9, 3.7 Hz, 4H), 3.35 (s, 4H), 2.49 (s, 3H). ¹⁹F NMR (471 MHz, DMSO) δ −129.46. LCMS (m/z): 464.1 (M+1), Rt: 2.741 min.

### 6-chloro-N-((4,5-difluoro-1H-benzo[d]imidazol-2-yl)methyl)-3-(5-methylthiophen-3-yl) imidazo[1,2-b]pyridazin-8-amine (95)

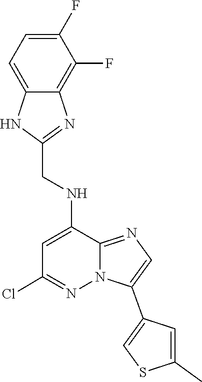 [SMILES: Cc1cc(-c2cnc3c(NCc4nc5c(F)c(F)ccc5[nH]4)cc(Cl)nn23)cs1]

Synthesized according to procedure described for 4. Yield (130 mg, 63.31%). Off-white solid. LCMS (m/z): 431.0 (M+1), Rt: 3.224 min.

### N-((4,5-difluoro-1H-benzo[d]imidazol-2-yl)methyl)-6-(4-methylpiperazin-1-yl)-3-(5-methylthiophen-3-yl) imidazo[1,2-b]pyridazin-8-amine (96)

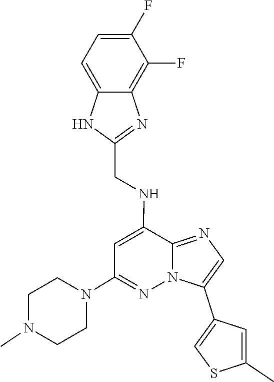

Synthesized according to procedure described for 5. Yield (12 mg, 20.91%), Off-white solid. ¹H NMR (500 MHz, DMSO) δ 12.72 (s, 1H), 8.16 (s, 1H), 8.03 (d, J=1.4 Hz, 1H), 7.75 (s, 1H), 7.57 (s, 1H), 7.40 (t, J=1.4 Hz, 1H), 7.26 (s, 1H), 7.23-7.18 (m, 1H), 6.08 (s, 1H), 4.82 (d, J=6.1 Hz, 2H), 3.40 (dd, J=6.3, 3.8 Hz, 4H), 2.49 (s, 3H), 2.42 (t, J=5.0 Hz, 4H), 2.21 (s, 3H). ¹⁹F NMR (471 MHz, DMSO) δ −150.11, −155.53. LCMS (m/z): 495.1 (M+1), Rt: 2.141 min.

### N-((4,5-difluoro-1H-benzo[d]imidazol-2-yl)methyl)-3-(5-methylthiophen-3-yl)-6-morpholinoimidazo[1,2-b]pyridazin-8-amine (97)

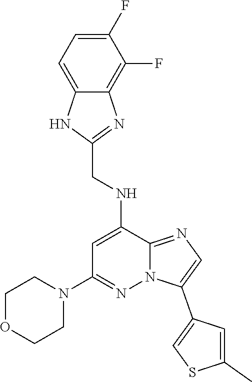 [SMILES: Cc1cc(-c2cnc3c(NCc4nc5c(F)c(F)ccc5[nH]4)cc(N4CCOCC4)nn23)cs1]

Synthesized according to procedure described for 5. Yield (6 mg, 10.74%), Off-white solid. ¹H NMR (500 MHz, DMSO) δ 12.71 (s, 1H), 8.05 (d, J=1.4 Hz, 1H), 7.77 (s, 1H), 7.61 (s, 1H), 7.40 (t, J=1.4 Hz, 1H), 7.27-7.16 (m, 2H), 6.09 (s, 1H), 4.83 (d, J=6.1 Hz, 2H), 3.71 (t, J=4.8 Hz, 4H), 3.37 (t, J=4.9 Hz, 4H), 2.49 (s, 3H). ¹⁹F NMR (471 MHz, DMSO) δ −150.11, −155.50. LCMS (m/z): 482.1 (M+1), Rt: 2.870 min.

### 4-(6-chloro-8-ethoxyimidazo[1,2-b]pyridazin-3-yl)-3,5-dimethylisoxazole (3i)

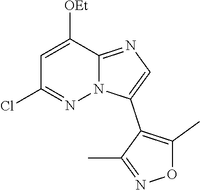 [SMILES: CCOc1cc(Cl)nn2c(-c3c(C)noc3C)cnc12]

Following the general procedure step 3, 3-bromo-6-chloro-8-ethoxyimidazo[1,2-b]pyridazine (1.0 g, 3.62 mmol), 3,5-dimethyl isoxazole boronic acid (560 mg, 3.98 mmol), K₂CO₃ (1.0 g, 7.23 mmol), 1,4-dioxane (8 mL) and water (2 mL) for 10 min. To this mixture, Pd(PPh₃)₄ (417 mg) was added, and the reaction was heated to 90° C. for 16 h. Yield (0.60 g, 56.68%), Pale-orange solid. LCMS (m/z): 293.0 (M+1), Rt: 2.503 min.

### 6-chloro-N-((5,6-dichloro-1H-benzo[d]imidazol-2-yl)methyl)-3-(3,5-dimethylisoxazol-4-yl)imidazo[1,2-b]pyridazin-8-amine (98)

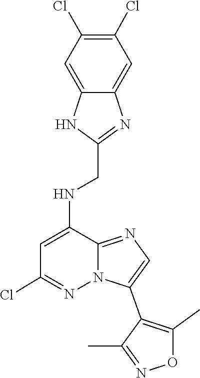 [SMILES: Cc1noc(C)c1-c1cnc2c(NCc3nc4cc(Cl)c(Cl)cc4[nH]3)cc(Cl)nn12]

Synthesized according to procedure described for 4. Yield (80 mg, 33.74%). Pale-yellow solid. LCMS (m/z): 464.0 (M+1), Rt: 3.010 min.

### N-((5,6-dichloro-1H-benzo[d]imidazol-2-yl)methyl)-3-(3,5-dimethylisoxazol-4-yl)-6-(4-methylpiperazin-1-yl) imidazo[1,2-b]pyridazin-8-amine (99)

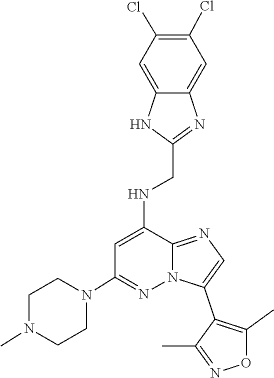

Synthesized according to procedure described for 5. Yield (2.80 mg, 6.15%), Pale-brown solid. ¹H NMR (500 MHz, DMSO) δ 7.78 (s, 2H), 7.64 (t, J=6.2 Hz, 1H), 7.48 (s, 1H), 6.02 (s, 1H), 4.82 (d, J=6.2 Hz, 2H), 3.54-3.48 (m, 4H), 3.27 (t, J=5.0 Hz, 4H), 2.38 (s, 3H), 2.16 (d, J=1.8 Hz, 6H). LCMS (m/z): 527.1 (M+1), Rt: 2.132 min.

### N-((5,6-dichloro-1H-benzo[d]imidazol-2-yl)methyl)-3-(3,5-dimethylisoxazol-4-yl)-6-morpholinoimidazo[1,2-b]pyridazin-8-amine (100)

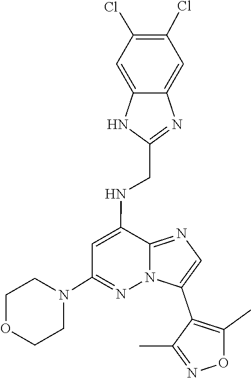 [SMILES: Cc1noc(C)c1-c1cnc2c(NCc3nc4cc(Cl)c(Cl)cc4[nH]3)cc(N3CCOCC3)nn12]

Synthesized according to procedure described for 5. Yield (3.50 mg, 7.89%), Off-white solid. ¹H NMR (500 MHz, DMSO) δ 8.46 (8, 1H), 7.77 (s, 2H), 7.68 (t, J=6.2 Hz, 1H), 7.50 (s, 1H), 6.04 (s, 1H), 4.82 (d, J=6.1 Hz, 2H), 3.64 (t, J=4.8 Hz, 4H), 3.24 (t, J=4.8 Hz, 4H), 2.38 (s, 3H), 2.20 (s, 3H). LCMS (m/z): 514.1 (M+1), Rt: 2.686 min.

### Example 2. Biological Activity

TABLE 2
Inhibition of cyclin-dependent kinase 12 and antiproliferative
activity of selected compounds of the present invention.

| | % Enzyme | | MDA-MB- | CCNK | |
|---|---|---|---|---|---|
| | Activity | CDK12 | 231 cell line | degradation | |
| Cmpd | CDK12@ 1 | (IC₅₀ | (GI₅₀ in | (DC₅₀ in | Dmax |
| # | uM | in nM) | nM) | nM) | (%) |
| 5 | — | 3000 | 4277 | 1300 | 65 |
| 6 | — | 3000 | 3840 | 460 | 75 |
| 8 | — | 10000 | 424 | 1300 | 100 |
| 9 | — | 9560 | 564 | 680 | 95 |
| 311 | — | 1000 | 7930 | 5600 | 60 |
| 12 | — | 1000 | 5689 | 2100 | 65 |
| 14 | 84 | 5000 | 1773 | 10000 | — |
| 15 | 68 | 3000 | 4110 | 2100 | 65 |
| 17 | 45 | 919 | 5601 | 10000 | — |
| 18 | 48 | 997 | 9922 | 1800 | 70 |
| 20 | 30 | 531 | 6674 | 10000 | — |
| 21 | 34 | 722 | 10000 | 10000 | — |
| 23 | 53 | 1110 | 5181 | 10000 | — |
| 24 | 60 | 1273 | 10000 | 2100 | 65 |
| 26 | 53 | 1107 | 6069 | 10000 | — |
| 27 | 52 | 1082 | 10000 | 1600 | 50 |
| 29 | 81 | — | 1125 | 10000 | — |
| 30 | 83 | — | 2125 | 3000 | 70 |
| 32 | 45 | 2079 | 4427 | 1400 | 50 |
| 33 | 60 | — | 6156 | 830 | 65 |
| 35 | 49 | — | 5168 | 1300 | 80 |
| 36 | 45 | 1223 | 4667 | 830 | 65 |
| 38 | 53 | — | 5433 | 1600 | 65 |
| 39 | 61 | — | 10000 | 920 | 80 |
| 41 | — | 1000 | 247 | 470 | 100 |
| 42 | — | 36 | 127 | 180 | 100 |
| 43 | — | 292 | 49 | 150 | 100 |
| 44 | — | 1419 | 154 | 440 | 100 |
| 45 | — | 567 | 365 | 1200 | 100 |
| 46 | — | 68 | 25 | 180 | 100 |
| 47 | — | 18 | 7 | 30 | 100 |
| 48 | — | 2445 | 130 | 200 | 100 |
| 49 | — | — | 465 | 370 | 95 |
| 51 | 55 | — | 5130 | 1900 | 50 |
| 52 | 23 | 359 | 390 | 30 | 80 |
| 54 | — | 175 | 440 | 110 | 85 |
| 55 | — | 283 | 472 | 70 | 80 |
| 57 | — | 54 | 419 | 240 | 65 |
| 58 | — | 65 | 406 | 260 | 100 |
| 60 | — | 907 | 284 | 1400 | 100 |
| 61 | — | 1320 | 235 | 590 | 100 |
| 63 | — | 2010 | 78 | 180 | 100 |
| 64 | — | 4250 | 168 | 340 | 100 |
| 66 | — | 90 | 180 | 80 | 85 |
| 67 | — | 300 | 207 | 80 | 85 |
| 68 | — | 65 | 33 | 10 | 90 |
| 69 | — | 57 | 92 | 150 | 85 |
| 70 | — | 78 | 73 | 160 | 95 |
| 71 | — | 130 | 71 | 160 | 100 |
| 72 | — | 56 | 49 | 50 | 100 |
| 73 | — | 1000 | 5000 | — | — |
| 75 | — | 38 | 146 | 140 | 80 |
| 76 | — | 7380 | 376 | 250 | 75 |
| 78 | 24 | 473 | 425 | 260 | 80 |
| 79 | 80 | 5000 | 1467 | 1700 | 75 |
| 80 | 75 | 3000 | 867 | 1000 | 95 |
| 81 | 33 | 647 | 1921 | 300 | 80 |
| 82 | 30 | 405 | 1115 | 250 | 50 |
| 84 | 61 | — | 2209 | 1500 | 80 |
| 85 | 73 | — | 3909 | 1200 | 85 |
| 87 | 82 | — | 7361 | 10000 | — |
| 88 | 82 | — | 460 | 1800 | 100 |
| 90 | 36 | 1408 | 4155 | 10000 | — |
| 91 | 103 | — | 3938 | 10000 | — |
| 93 | 36 | 1216 | 1246 | 80 | 80 |
| 94 | 70 | — | 1495 | 1000 | 80 |
| 96 | 63 | — | 3110 | 1800 | 75 |
| 97 | 79 | — | 10000 | 1300 | 75 |
| 99 | 59 | 1253 | 689 | 10000 | — |
| 100 | 73 | 3000 | 2031 | 10000 | — |

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A compound of Formula I 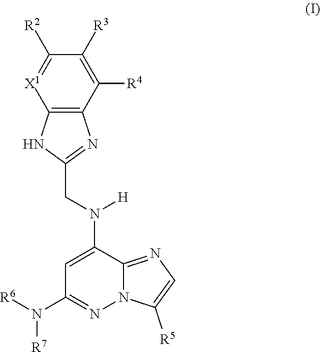
or a pharmaceutically acceptable salt or derivative thereof;
wherein:
X¹ is CR¹ or N;
R¹, R², R³ and R⁴ are independently selected from hydrogen, halo, cyano, azido, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, each of which may be optionally substituted with one or more groups selected from Y as allowed by valency;
R⁵ is 5- to 10-membered monocyclic or bicyclic heteroaryl optionally substituted with one or more groups selected from Z as allowed by valency;
R⁶ is selected from C₁-C₆ alkyl and 3- to 8-membered monocyclic or bicyclic heterocycle optionally substituted with one or more groups selected from Z;
R⁷ is selected from hydrogen and C₁-C₆ alkyl;
or R⁶ and R⁷ are brought together with the nitrogen to which they are attached to form a 3- to 8-membered monocyclic or bicyclic heterocycle optionally substituted with one or more groups selected from Z;
Z is independently selected at each occurrence from hydrogen, halo, cyano, azido, oxo, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, each of which may be optionally substituted with one or more groups selected from Y as allowed by valency;
R^{z} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

2. The compound of claim 1, or a pharmaceutically acceptable salt or derivative thereof, wherein X¹ is CR¹.

3. The compound of claim 1, or a pharmaceutically acceptable salt or derivative thereof, wherein X¹ is N.

4. The compound of claim 1, or a pharmaceutically acceptable salt or derivative thereof, wherein R¹, R², R³, and R⁴ where present are independently selected from hydrogen, halo, and C₁-C₆ alkyl.

5. The compound of claim 1, or a pharmaceutically acceptable salt or derivative thereof, wherein R¹, R², R³, and R⁴ where present are independently selected from hydrogen, chloro, fluoro, and methyl.

6. The compound of claim 1, or a pharmaceutically acceptable salt or derivative thereof, wherein X¹ is CR¹, R¹ and R⁴ are hydrogen, and R² and R³ are chloro, or
X¹ is CR¹, R¹ is chloro and R², R³, and R⁴ are hydrogen, or
X¹ is N, and R² and R³ are hydrogen, and R⁴ is methyl, or
X¹ is CR¹, R¹, R², and R³ are hydrogen, and R⁴ is fluoro, or
X¹ is CR¹, R¹ and R² are hydrogen, and R³ and R⁴ are fluoro.

7-10. (canceled)

11. The compound of claim 1, or a pharmaceutically acceptable salt or derivative thereof, wherein R⁵ is 5-membered monocyclic heteroaryl having one or two heteroatoms independently selected from N, O, and S and optionally substituted with one or two groups selected from Z as allowed by valency.

12. The compound of claim 1, or a pharmaceutically acceptable salt or derivative thereof, wherein R⁵ is selected from pyrazolyl, thienyl, and isoxazolyl optionally substituted with or two groups selected from Z as allowed by valency.

13. The compound of claim 1, or a pharmaceutically acceptable salt or derivative thereof, wherein R⁵ is selected from:

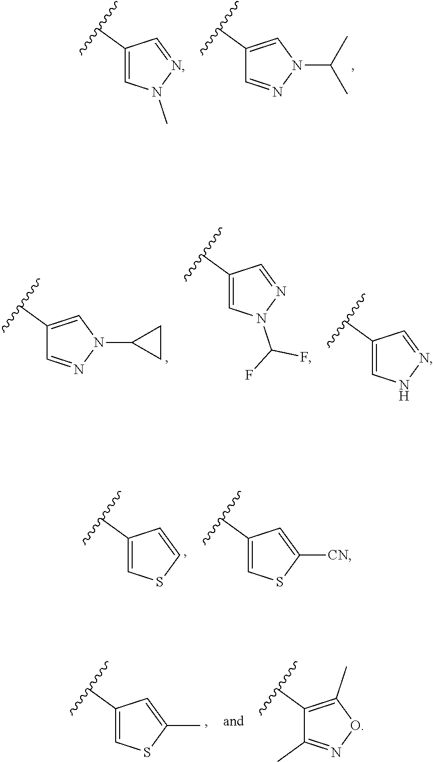

14. The compound of claim 1, or a pharmaceutically acceptable salt or derivative thereof, wherein R⁷ is hydrogen, and R⁶ is 5- to 6-membered monocyclic heterocyclic having one or two heteroatoms independently selected from N, O, and S and optionally substituted with one or two groups selected from Z as allowed by valency, or
wherein R⁶ and R⁷ are brought together with the nitrogen to which they are attached to form a 5- to 6-membered monocyclic heterocycle optionally substituted with one or two groups selected from Z as allowed by valency.

15. The compound of claim 1, or a pharmaceutically acceptable salt or derivative thereof, wherein R⁷ is hydrogen and R⁶ is selected from piperazinyl, morpholinyl, or piperidinyl optionally substituted with one or two groups selected from Z as allowed by valency, or
wherein R⁶ and R⁷ are brought together with the nitrogen to which they are attached to form a piperazinyl, morpholinyl, or piperidinyl ring optionally substituted with one or two groups selected from Z as allowed by valency.

16-17. (canceled)

18. The compound of claim 1, or a pharmaceutically acceptable salt or derivative thereof, wherein —NR⁶R⁷ is selected from:

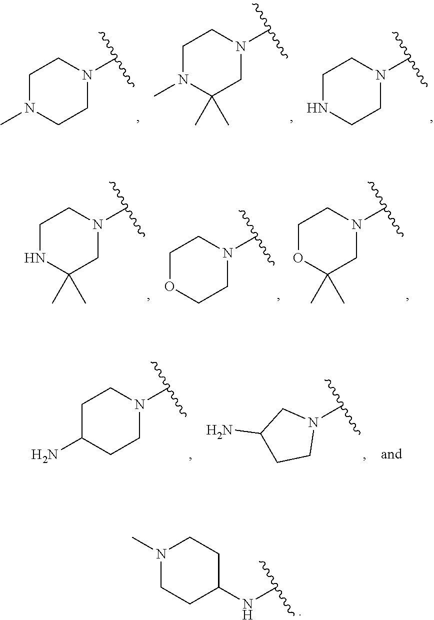

19. A compound of claim 1 selected from 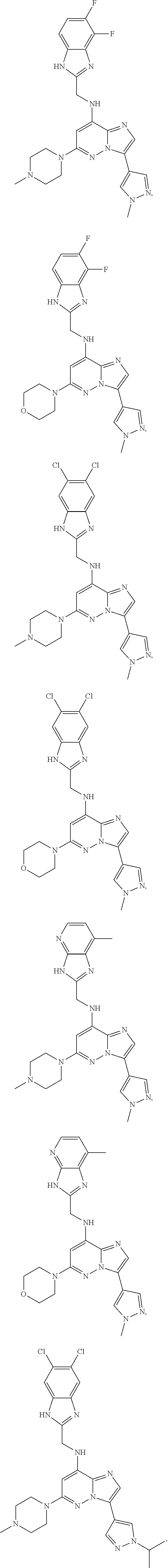 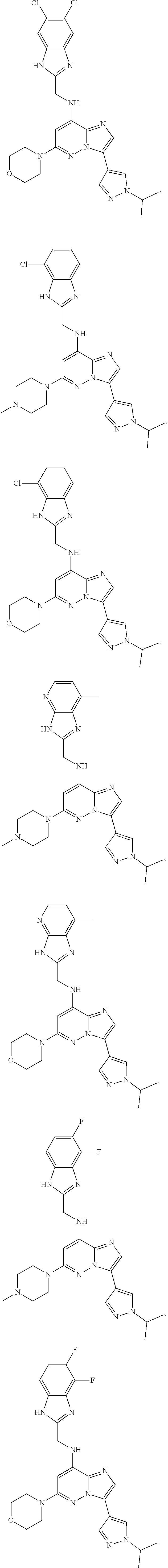 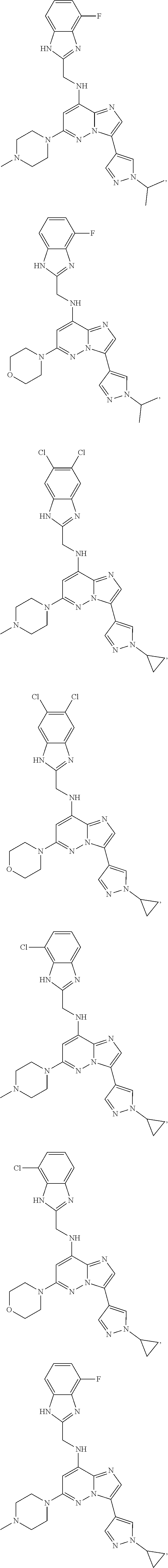 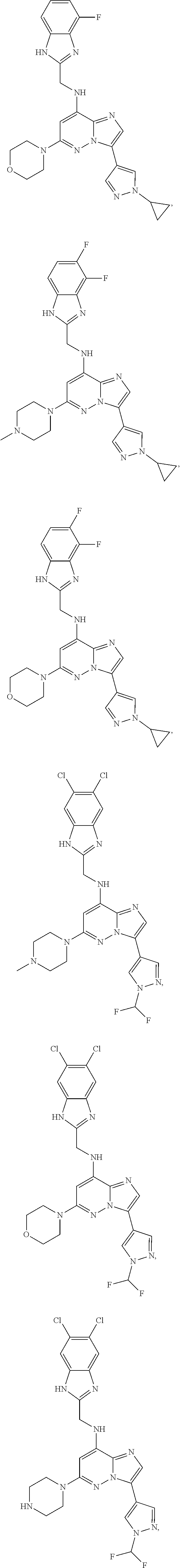 [SMILES: Fc1cccc2[nH]c(CNc3cc(N4CCOCC4)nn4c(-c5cnn(C6CC6)c5)cnc34)nc12 ; CN1CCN(c2cc(NCc3nc4c(F)c(F)ccc4[nH]3)c3ncc(-c4cnn(C5CC5)c4)n3n2)CC1 ; Fc1ccc2[nH]c(CNc3cc(N4CCOCC4)nn4c(-c5cnn(C6CC6)c5)cnc34)nc2c1F ; CN1CCN(c2cc(NCc3nc4cc(Cl)c(Cl)cc4[nH]3)c3ncc(-c4cnn(C(F)F)c4)n3n2)CC1 ; FC(F)n1cc(-c2cnc3c(NCc4nc5cc(Cl)c(Cl)cc5[nH]4)cc(N4CCOCC4)nn23)cn1 ; FC(F)n1cc(-c2cnc3c(NCc4nc5cc(Cl)c(Cl)cc5[nH]4)cc(N4CCNCC4)nn23)cn1] 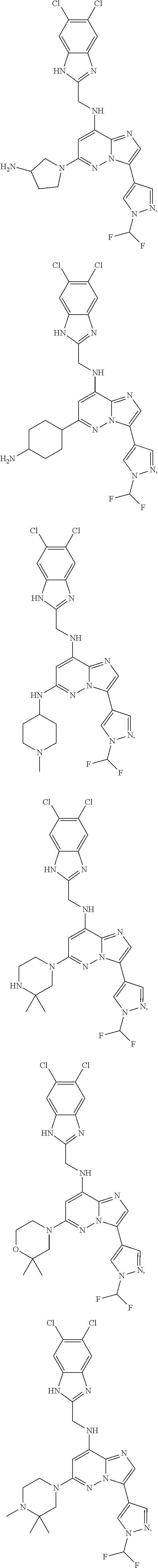 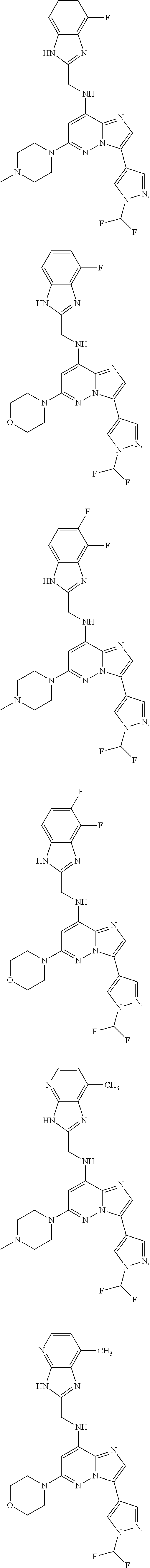 [SMILES: CN1CCN(c2cc(NCc3nc4c(F)cccc4[nH]3)c3ncc(-c4cnn(C(F)F)c4)n3n2)CC1 ; Fc1cccc2[nH]c(CNc3cc(N4CCOCC4)nn4c(-c5cnn(C(F)F)c5)cnc34)nc12 ; CN1CCN(c2cc(NCc3nc4c(F)c(F)ccc4[nH]3)c3ncc(-c4cnn(C(F)F)c4)n3n2)CC1 ; Fc1ccc2[nH]c(CNc3cc(N4CCOCC4)nn4c(-c5cnn(C(F)F)c5)cnc34)nc2c1F ; Cc1ccnc2[nH]c(CNc3cc(N4CCN(C)CC4)nn4c(-c5cnn(C(F)F)c5)cnc34)nc12 ; Cc1ccnc2[nH]c(CNc3cc(N4CCOCC4)nn4c(-c5cnn(C(F)F)c5)cnc34)nc12] 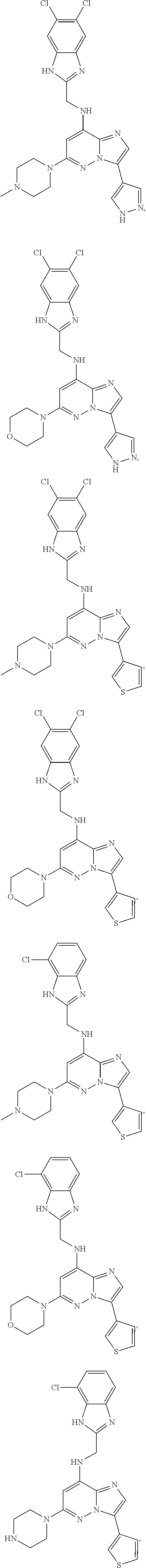 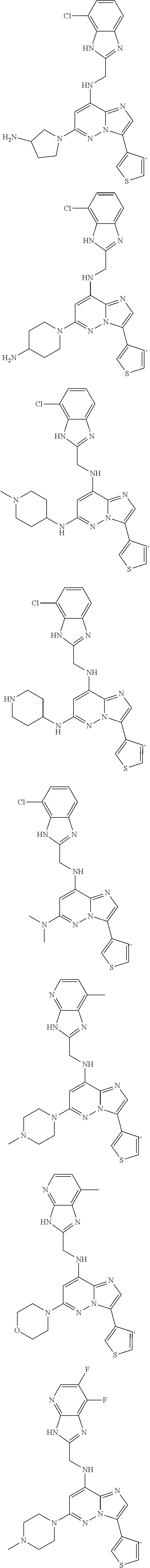 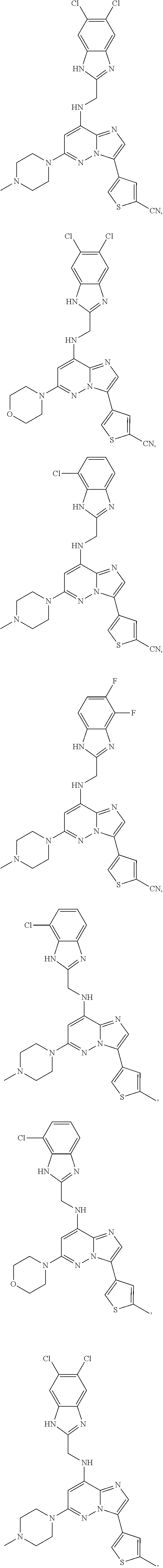 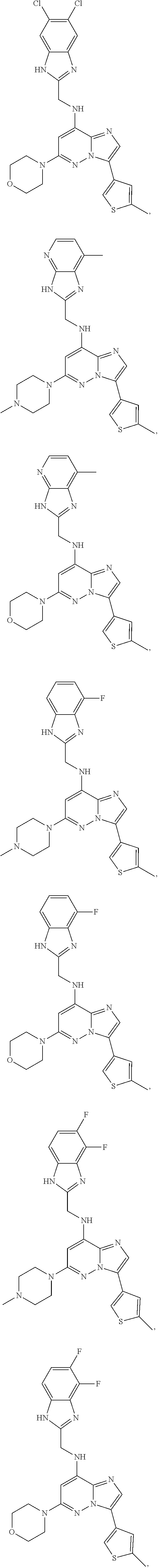 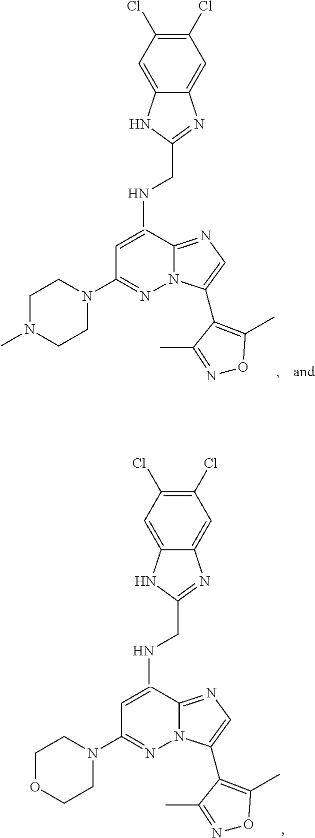
or a pharmaceutically acceptable salt or derivative thereof.

20. A pharmaceutical composition comprising a compound of claim 1, or a pharmaceutically acceptable salt or derivative thereof, and a pharmaceutically acceptable carrier or excipient.

21. A method of inhibiting a cyclin-dependent kinase, comprising contacting the cyclin dependent-kinase with an effective amount or concentration of a compound of claim 1, or a pharmaceutically acceptable salt or derivative thereof.

22. The method of claim 21, wherein the cyclin-dependent kinase is cyclin-dependent kinase 12 or 13 (CDK12 or CDK13).

23. (canceled)

24. A method of treating a disorder of uncontrolled cellular proliferation or a disease selected from inflammatory or myotonic dystrophy type diseases in a mammal comprising administering to the mammal an effective amount of a compound of claim 1, or a pharmaceutically acceptable salt thereof.

25-26. (canceled)

27. The method of claim 24, wherein the disorder of uncontrolled cellular proliferation is cancer.

28. The method of claim 27, wherein the cancer is selected from breast cancer, brain cancer, cervical cancer, chronic myeloproliferative disorder, colorectal cancer, Ewing's sarcoma, gastrointestinal cancer, glioma, leukemia, lung cancer, lymphoma, endometrial cancer, melanoma, multiple myeloma, myelodysplastic syndrome, myeloproliferative neoplasm, pancreatic cancer, plasma cell neoplasm (myeloma), prostate cancer, ovarian cancer, osteosarcoma, skin cancer, testicular cancer, and thyroid cancer.

29-30. (canceled)

31. The method of claim 24, wherein the mammal is diagnosed with inflammatory or myotonic dystrophy type 1 disease.
